(19) United States
(12) Patent Application Publication
(10) Pub. No.: US 2012/0214987 A1
(43) Pub. Date: Aug. 23, 2012

(54) METHODS AND COMPOUNDS FOR PREPARING 3ALPHA-OXYGEN SUBSTITUTED STEROIDS

(76) Inventors: Yu Ge, San Diego, CA (US); Steven K. White, San Diego, CA (US); Yujin Huang, San Diego, CA (US)

(21) Appl. No.: 13/327,701

(22) Filed: Dec. 15, 2011

Related U.S. Application Data
(60) Provisional application No. 61/423,457, filed on Dec. 15, 2010.

(51) Int. Cl. C07J 71/00; C07J 3/00

(52) U.S. Cl. 540/23; 540/76; 552/650; 552/612

(57) ABSTRACT

The invention relates to processes for preparing 3α-O-linked steroids including 3α-O-linked-androst-5-ene steroids and 3α-O-linked-5a-androstane steroids. In one process a 3α,4α-epoxy androst-5-en-17-one is predominately reduced at the epoxy moiety wherein reduction of the 3α,4α epoxy functional group occurs preferentially at position C4 with retention of configuration at position C3 to provide a 3α-O-linked-androst-5-ene steroid. In another process, conditions are provided for inversion of configuration of a 3β-hydroxy-androst-5-ene steroid by the Mitsunobu reaction to provide a 3α-O-linked-androst-5-ene steroid with reduced amounts of 3α,5α-cycloandrostane side-product impurities.

### CROSS REFERENCE TO RELATED APPLICATIONS

This nonprovisional U.S. patent application claims priority under 35 USC §119(e) from pending U.S. provisional application No. 61/423,457, filed on Dec. 15, 2010, which is incorporated herein by reference in its entirety.

### FIELD OF THE INVENTION

Invention embodiments relate to new methods for preparing 3α-hydroxy steroids and related compounds, such as ester or ether derivatives thereof. Invention embodiments further relate to preparation of and use of intermediates, such as 3α-hydroxy-androst-5-en-17-one (3α-DHEA) and 3α-hydroxy-androst-5-en-7,17-dione, to make such steroids.

### BACKGROUND

Steroids having a monovalent oxygen-linked substituent in the α-configuration, such as a 3α-hydroxy substituent, e.g., 3α-hydroxyandrost-5-enes and 3α-hydroxy-5α-androstanes, have not been prepared at reaction scales typically used in the development or manufacturing of approved pharmaceutical compounds. Such compounds are sometimes used in small scale research, which requires smaller amounts of material.

The present invention provides new methods that can be used to make compounds such as 3α-hydroxyandrost-5-enes and 3α-hydroxy-5α-androstanes at larger, non-research scales. Such larger scale syntheses are useful for supporting development of these compounds for human use, e.g., in clinical trial protocols or in large scale preclinical studies such as animal toxicology studies to support human uses. The new methods provide higher purity products and reduced synthesis costs.

### SUMMARY OF THE INVENTION

In some embodiments, the invention provides a method or process to prepare a 3α-O-linked steroid comprising the step of contacting a suitably protected 3α,4α-epoxyandrost-5-ene with a hydrogen donor wherein the 3α,4α epoxy functional group is selectively reduced relative to the Δ⁵ functional group and wherein reduction of the 3α,4α epoxy functional group occurs preferentially at position C-4 with retention of configuration at position C-3, whereby a 3α-hydroxy-androst-5-ene steroid product is obtained.

In some of these embodiments, the invention provides a process to prepare a 3α-O-linked steroid comprising (1) contacting a suitably protected 3α,4α-epoxyandrost-5-ene having the structure

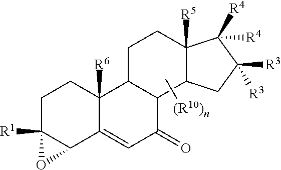

with a first hydrogen donor wherein the 3α,4α epoxy functional group is preferentially reduced relative to the Δ⁵ functional group and wherein reduction of the 3α,4α epoxy functional group occurs preferentially at position C-4 with retention of configuration at position C-3 with (e.g. >50%) or without (e.g., <50%) appreciable C-7 ketone reduction, wherein the first hydrogen donor optionally is an aluminum hydride or a palladium metal catalyst in the presence of hydrogen gas; and optionally (2) contacting the product of step (1) with an electrophile wherein a monovalent O-linked moiety is formed at position C3 or at positions C3 and C7, wherein the monovalent O-linked moiety(ies) are derived from the electrophile, whereby a 3α-O-linked-androst-5-en-7-one steroid or a 3α,7ζ-di-O-linked-androst-5-ene steroid is prepared, optionally after protecting group removal. In these embodiments the 3α,4α-epoxyandrost-5-ene reacted in step (1) has substituents R¹ is —H or a suitably protected optionally substituted alkyl, optionally C₁₋₄ alkyl or a suitably protected C₁₋₄ hydroxyalkyl, optionally —CH₃, —C₂H₅ or —CH—CH₂OR^{PR}, wherein R^{PR} is a protecting group, where the —OR^{PR} moiety defines, for example, an ester, ether or silylether such as —OC(O)CH₃, —OCH₃, —OSi(CH₃)₃; R³ independently are —H, a suitable halogen (i.e., does not undergo appreciable dehalogenation or dehydrohalogenation in the presence of the first hydrogen donor), a suitably protected —OH group (i.e., —OR^{PR}) or other monovalent O-linked moiety, optionally substituted, including an ester, ether or silylether such as —OC(O)CH₃, —OCH₃ or —OSi(CH₃)₃ or an optionally substituted monovalent C-linked moiety, optionally C₁₋₄ alkyl or a suitably protected C₁₋₄ hydroxyalkyl, optionally —CH₃, —C₂H₅, —CH₂CH₂CH₃ or —CHCH₂OR^{PR}, wherein R^{PR} is a protecting group, where the —OR^{PR} moiety defines, for example, an ester, ether or silylether such as —OC(O)CH₃, —OCH₃, —OSi(CH₃)₃; R⁴ independently are a monovalent O-linked moiety, optionally substitutes such as a suitably protected —OH (i.e., —OR^{P}) or other monovalent O-linked moiety including, ester, ether or silylether such as —OC(O)CH₃, —OCH₃ or —OSi(CH₃)₃, provided that R⁴ are not both —OH, or both of R⁴ together are —OC(R¹⁶)₂C(R¹⁶)₂O—, wherein R¹⁶ independently are optionally substituted alkyl or two of R¹⁶ and the carbon(s) to which they are attached comprise a cycloalkyl or spiroalkyl and the remaining R¹⁶ are independently optionally substituted alkyl, suitably protected; R⁵ and R⁶ independently are —H or optionally substituted alkyl, suitably protected, optionally C₁₋₄ alkyl or a suitably protected C₁₋₄ hydroxyalkyl, optionally —CH₃, —C₂H₅ or —CHCH₂OR^{PR}, wherein R^{PR} is a protecting group, where the —OR^{PR} moiety defines for example an ester, ether or silylether such as —OC(O)CH₃, —OCH₃, —OSi(CH₃)₃; (R¹⁰)ₙ—, is 0, 1, 2, 3 or 4 independently selected R¹⁰ substituents attached to the steroid ring replacing hydrogen, wherein the R¹⁰ substituents replace none, one, two, three or four positions selected from the group consisting of positions C-1, C-2, C-4, C-6, C-9, C-11, C-12 and C-15, wherein none, one or two R¹⁰ may be present at positions C-1, C-2, C-11 and C-15 and none or one R¹⁰ may be present at positions C-4, C-6 or C-9, wherein R¹⁰, if present at position C-9 is a halogen such as —Cl or —F, if present at positions C-4 or C-6, is independently selected optionally substituted alkyl or C₁₋₄ optionally substituted alkyl, suitably protected, such as —CH₃, —C₂H₅ or —CH₂CH₂OR^{PR}, wherein R^{PR} is a protecting group, where the —OR^{PR} moiety defines for example an ester, ether or silylether such as —OC(O)CH₃, —OCH₃, —OSi(CH₃)₃, and if present at positions C-1, C-2, C-11 or C-15 is independently selected halogen, such as —Cl or —F or an optionally monovalent C-linked moiety or an optionally substituted monovalent O-linked moiety, suitably protected.

In preferred embodiments, O-linked moieties of 3α,4α-epoxyandrost-5-enes, 3β-hydroxy-androstenes or their precursors or of products of the processes described herein are, —OH, —OR^{PR}, a C₂₋₆ ester, e.g. acetate or propionate, a silyl ether, e.g., trimethylsilyl ether or t-butyldimethylsilyl ether, or a C₁₋₆ alkyl ether, e.g., methyl ether, ethyl ether or tetrahydropyranyl ether, or are O-linked moieties as described in the claims. Preferred C-linked moieties are optionally substituted C₁₋₆ alkyl such as —CH₃, —CH₂CH₂OH, —CH₂CH₂OR^{PR}—C₂H₅, —CH₂CH₂CH₂OH, —CH₂CH₂CH₂OR^{PR} and —CH₂CH₂CH₃.

In some embodiments, the invention provides a method or process that contacts a suitably protected 3α,4α-epoxy-androst-5-ene steroid with a reducing agent that preferentially reduces the epoxy functional group such that an oxygen substituent at position C-3 in the α-configuration results.

In other embodiments, reaction conditions are described for transforming a 3β-hydroxy-androst-5-ene steroid into a 3α-O-linked-androst-5-ene steroid substantially free of 3α,5α-cycloandrostanes as process impurities. Prior methods for inverting configuration of 3β-hydroxy-androst-5-ene steroids at position C-3 provide significant amounts of these 3α,5α-cycloandrostane impurities. Conditions disclosed herein for inversion of configuration at position C-3 of 3β-hydroxyl-androst-5-ene steroid unexpectedly provide a 3α-hydroxy-androst-5-ene steroid substantially free of 3α,5α-cycloandrostane steroid impurities.

In other embodiments processes are provided that use 3α-O-linked-androst-5-ene steroid products as precursors for preparation of 3α-O-linked-5α-androstanes.

In additional embodiments processes are provided for preparation of 3α-O-linked-androst-5-enes and 3α-O-linked-5α-androstanes having disubstitution at position C-17 using 3α-O-linked-androst-5-ene and 3α-O-linked-5α-androstane precursors with C-17 monosubstitution that are products of the processes described herein.

In some principle embodiments processes are described herein that are useful for preparing biologically active 3α-hydroxy-androst-5-enes and 3α-hydroxy-androstanes. In other principle embodiments intermediates useful in preparation of those biologically active compounds are provided.

### DETAILED DESCRIPTION

### Definitions

As used herein and unless otherwise stated or implied by context, terms that are defined herein have the meanings that are specified. The descriptions of embodiments and examples that are described illustrate the invention and they are not intended to limit it in any way. Unless otherwise contraindicated or implied, e.g., by including mutually exclusive elements or options, in these descriptions and throughout this specification, the terms “a” and “an” mean one or more and the term “or” means and/or.

“Alkyl” as used here refers to moieties with linked normal, secondary, tertiary or cyclic carbon atoms, i.e., linear, branched, cyclic or any combination thereof. Alkyl groups or moieties, as used herein, may be saturated, or unsaturated, i.e., the moiety may comprise one, two, three or more independently selected double bonds or triple bonds. Unsaturated alkyl moieties are as described below for alkenyl, alkynyl, cycloalkenyl and aryl moieties. Substituted alkyl moieties may be substituted with moieties as described below for alkenyl, alkynyl, cycloalkyl, aryl, heteroaryl and heterocycle moieties. The number of carbon atoms in an alkyl moiety is typically 1 to about 10. Expressions such as C₁₋₆ alkyl or C₁₋₆ alkyl mean an alkyl moiety containing 1, 2, 3, 4, 5 or 6 carbon atoms. When an alkyl group or substituent is specified, species include, e.g., methyl, ethyl, 1-propyl (n-propyl), 2-propyl (iso-propyl, —CH(CH₃)₂), 1-butyl (n-butyl), 2-methyl-1-propyl (iso-butyl, —CH₂CH(CH₃)₂), 2-butyl (sec-butyl, —CH(CH₃)CH₂CH₃) and 2-methyl-2-propyl (t-butyl, —C(CH₃)₃). Preferred alkyl groups are C₁₋₈ alkyl with C₁₋₆ and C₁₋₄ alkyl moieties particularly preferred and methyl and ethyl more preferred species.

“Cycloalkyl” as used here refers to an alkyl moiety that comprises a non-aromatic monocyclic, bicyclic or tricyclic ring system composed of only carbon atoms. The number of carbon atoms in a cycloalkyl group or moiety can vary but typically this number is 3 to about 10. C₃₋₆ alkyl or C₃₋₆ alkyl means a cycloalkyl moiety containing 3, 4, 5 or 6 carbon atoms. Cycloalkyl moieties having a double bond within the cyclic ring system are sometimes referred to as cycloalkenyl moieties. Substituted cycloalkyl moieties may be substituted through one of its carbon atoms through a double or single bond with moieties as described for alkyl, alkenyl, alkynyl, aryl, heteroaryl and heterocycle moieties. Substituted cycloalkyl moieties may also be substituted through two of its carbon atoms through single and/or double bonds with moieties as described for alkyl, alkenyl, alkynyl, aryl, heteroaryl and heterocycle moieties to form a bicyclic ring system. When a cycloalkyl group or substituent is specified, species include, e.g., cyclopropyl, cyclopentyl, cyclohexyl, cycloheptyl or adamantyl with cyclopentyl and cyclohexyl preferred.

“Alkenyl” as used here means a moiety or group that comprises one or more double bonds (—CH═CH—), e.g., 1, 2, 3, 4 or more, typically 1 or 2 and include an aryl moiety such as phenyl. Alkenyl moieties may be additionally comprised of linked normal, secondary, tertiary or cyclic carbon atoms, i.e., linear, branched, cyclic or any combination thereof. An alkenyl moiety with multiple double bonds may have the double bonds arranged contiguously (e.g., a 1,3-butadienyl moiety) or non-contiguously with one or more intervening saturated carbon atoms or a combination thereof. The number of carbon atoms in an alkenyl moiety typically is 2 to about 10. C₂₋₆ alkenyl or C₂₋₆ alkenyl means an alkenyl moiety containing 2, 3, 4, 5 or 6 carbon atoms. Substituted alkenyl moieties may be substituted with moieties as described below for alkyl, cycloalkyl, alkynyl, aryl, heteroaryl and heterocycle moieties. When an alkenyl group or substituent is specified, species include, e.g., any of the alkyl moieties that have an internal double bond such as vinyl (—CH═CH₂), allyl (—CH═CHCH₃), 1-methylvinyl, butenyl, iso-butenyl, 3-methyl-2-butenyl or 1-pentenyl and moieties with a terminal double bond, such as methylene (═CH₂), methylmethylene (═CH—CH₃), ethylmethylene (═CH—CH₂—CH₃) or propylmethylene (═CH—CH₂—CH₂—CH₃). Preferred alkenyl moieties are C₂₋₈ alkenyl, with C₂₋₆ and C₂₋₄ alkenyl moieties particularly preferred.

“Alkynyl” as used herein refers to linked normal, secondary, tertiary or cyclic carbon atoms where one or more triple bonds (—C≡C—) are present, typically 1, 2 or 3, usually 1, optionally also having 1, 2 or more double bonds, with the remaining bonds (if present) being single bonds to linked normal, secondary, tertiary or cyclic carbon atoms, i.e., linear, branched, cyclic or any combination thereof. The number of carbon atoms in an alkynyl group or moiety is typically 2 to about 10. C₂₋₆ alkynyl or C₂₋₆ alkynyl means an alkynyl moiety containing 2, 3, 4, 5 or 6 carbon atoms. Substituted alkynyl moieties may be substituted with moieties as described below for alkyl, alkenyl, aryl and heteroaryl moieties. When an alkynyl group or substituent is specified, species include any of the alkyl moieties incorporating a terminal triple bond such as —C≡CH, —C≡CCH₃, —C≡CCH₂CH₃, —C≡CC₃H₇ or —C≡CCH₂C₃H₇. Preferred alkynyl moieties are C₂₋₈ alkynyl with C₂₋₆ and C₂₋₄ alkynyl particularly preferred and species ethynyl, 1-propynyl and 1-butynyl particularly preferred with ethynyl especially preferred.

“Aryl” as used herein refers to an aromatic ring system or a fused ring system containing no ring heteroatoms and comprising 1, 2 or 3 rings, typically 1 or 2 rings, some of which may participate in exocyclic conjugation (i.e., cross-conjugated). When an aryl group or substituent is specified, species include, e.g., phenyl, biphenyl, naphthyl, phenanthryl or quinone, with phenyl preferred. Substituted aryl moieties may be substituted with moieties that are as described below for alkyl, cycloalkyl, alkenyl, alkynyl, heteroaryl and heterocycle moieties.

“Heteroaryl” as used here refers means an aryl ring system wherein one or more, typically 1, 2 or 3, but not all of the carbon atoms comprising the aryl ring system are replaced independently by a heteroatom, which is a heavy atom other than carbon, including, N, O, S, Se, B, Si, P, typically, oxygen (—O—), nitrogen (—NX—) or sulfur (—S—) where X is —H, a protecting group or C₁₋₆ optionally substituted alkyl, wherein the heteroatom participates in the conjugated system either through pi-bonding with an adjacent atom in the ring system or through a lone pair of electrons on the heteroatom. The aryl ring system may be optionally substituted on one or more its carbons or heteroatoms, or a combination of both, in a manner which retains the cyclically conjugated system. A heteroaryl substituent attached to an organic moiety, such as an androst-5-ene or 5α-androstane steroid, through a carbon of the heteroaryl aromatic ring system is referred to as a C-linked heteroaryl or C-heteroaryl.

“Heterocycle” or “heterocyclic” includes by way of example and not limitation the heterocycles described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 1960, 82:5566. A heterocycle group or substituent is typically bonded to an organic moiety through a ring carbon atom or a ring nitrogen atom of the heterocycle. Heterocycle groups or substituents include aromatic (i.e., heteroaryl) and non-aromatic heterocycles. A heterocycle substituent attached to an organic moiety, such as an androst-5-ene or 5α-androstane steroid, through a carbon of the heterocyclic ring system is referred to as a C-linked heterocycle or a C-heterocycle and a heterocycle bonded through a nitrogen atom of the heterocyclic ring is referred to as an N-linked heterocycle or an N-heterocycle. Preferred heterocycles are morpholine, piperidine, pyrazine, pyrimidine, pyrrolidine, imidazole and pyrazole. For certain preferred heterocycle substituents, a C-heterocycle or an N-heterocycle is preferably bonded to the 17-position or the 11-position of the steroid compounds described herein, e.g., 1113-N-morpholino or 1713-(4′-imidazolyl).

“Sprioalkyl” as used here refers to a cycloalkyl or heterocycle group that is bonded through single bonds to another cycloalkyl or heterocyle through one carbon atom shared between the cycloalkyl and/or heterocylc moieties. Preferred spiroalkyl groups or moieties have the structure

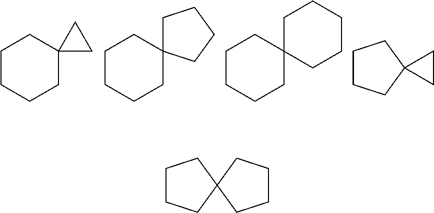

where one or more of the non-shared carbon atoms (preferrably one or two) may be replaced independently with a heteroatom such as N, O or S. Preferred spiroalkly groups having such substitutions have the structure

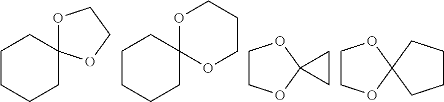

“Protecting group” as used here means a moiety that prevents or inhibits the atom or functional group to which it is linked from participating in unwanted reactions. For example, for —OR^{PR}, R^{PR} is a protecting group for the oxygen atom found in a hydroxyl, while for ═O (ketone), the protecting group is a ketal or thioketal wherein the divalent oxygen is replaced, for example, in cyclic ketals or cyclic thioketals by —X—[C(R¹⁶)₂], —Y—, wherein X and Y independently are S and O; n is 2 to 3 to form a heterocyclic ring system defined by X, Y and the carbon of the ketone so protected; and R¹⁶ independently are —H or alkyl or two of R¹⁶ together with the carbons to which they are attached define a cycloalkyl or spiroalkyl, where the remaining R¹⁶ are independently —H or alkyl, or two of R¹⁶ together form an o-catechol, where the remaining R¹⁶ are replaced by a double bond, or the protecting group is an oxime wherein ═O is replaced by ═N—OR¹¹, wherein R¹¹ is as defined for ether or silyl ether. Preferred R¹¹ for oximes moieties are —H, alkyl or —Si(R¹³)₃, with R¹³ as defined for silyl ether. Ketals also include cyclic ketals that contain structures such as —O—C(R¹⁶)₂—C(R¹⁶)₂O—, wherein R¹⁶ retains its previously defined meaning. For —C(O)—OR^{PR}, R^{PR} is a carbonyloxy protecting group, for —SR^{PR}, R^{PR} is a protecting group for sulfur in thiols, for instance, and for —NHR^{PR} or —N(R^{PR})₂—, R^{PR} independently selected is a nitrogen atom protecting group for primary or secondary amines. The protecting groups for sulfur or nitrogen are usually used to avoid unwanted reactions with electrophilic compounds. The protecting groups for oxygen are used to avoid unwanted reactions with electrophiles, and are typically esters (e.g. acetate, propionate or benzoate), or avoid interfering with the nucleophilicity of organometallic reagents or other highly basic reagents, and are typically ethers, optionally substituted, including alkyl ethers, (e.g., methyl or tetrahydropyranyl ethers) alkoxymethyl ethers (e.g., methoxymethyl or ethoxymethyl eters), optionally substituted aryl ethers and silyl ethers (e.g. trimethylsilyl (TMS), triethylsilyl (TES), tert-butyldiphenylsilyl (TBDPS), tert-butyldimethylsilyl (TBS/TBDMS), triisopropylsilyl (TIPS) and [2-(trimethylsilyl)ethoxy]methylsilyl (SEM)).

The divalent oxygen moiety ═O (ketone) is usually protected with protecting groups that avoid unwanted reactions with nucleophilic compounds and typically is a ketal, a thioketal, a cyclic ketal or a cyclic thioketal, with cyclic ketal preferred, or the ketone is masked in reduced form as a suitably protected hydroxyl group. Preferred hydroxyl protecting groups are methoxymethyl (i.e., hydroxy protected as a substituted ether), acetyl (i.e., hydroxyl protected as acetate ester), acyl (for example, hydroxyl protected as propionate or benzoate ester) and (R¹³)₃Si—, wherein R¹³ independently are as defined for silyloxy (i.e., hydroxyl protected as a silyl ether), with protection as acetate ester, trimethylsilyl ether and t-butyldimethylsilyl ether preferred. A preferred ketone protecting group is the divalent O-linked moiety —O—CH₂—CH₂—O— (ketal), which can be used to protect a ketone as its cyclic ketal at the 17-position or the 7-position of a steroid, such as an androst-5-en-7-one, androst-5-en-17-one, 5α-androstan-7-one steroid or a 5α-androstan-17-one.

“Optionally substituted alkyl”, “optionally substituted alkenyl”, “optionally substituted alkynyl”, “optionally substituted heterocycle”, “optionally substituted aryl”, “optionally substituted heteroaryl” and the like mean an alkyl, cycloalkyl alkenyl, alkynyl, aryl, heteroaryl, heterocycle or other group or moiety as defined or disclosed herein that has a substituent(s) that optionally replaces a hydrogen atom(s) in the group or moiety. Such substituents are as described above. For an optionally substituted phenyl moiety (-Ph), the arrangement of any two substituents present on the aromatic ring can be ortho (O), meta (m), or para (p) to each other. Preferred optionally substituted moieties are optionally substituted phenyl, including -Ph-NO₂ and -Ph-halogen, wherein halogen is —F, —Br, —Cl or —I, with —Br and —F preferred, optionally substituted alkyl, including —CH₂Ph, —CF₃, —CH₂OH, —CH₂-halogen, wherein -halogen is —F, —Br, —Cl or —I, with —I or —Br preferred, and optionally substituted alkynyl, including —C≡CCH₂OH, —C≡C-halogen, with C≡C—Cl preferred, —C≡C—Si(R¹³)₃, with R¹³ as previously defined for silyl ether, with —C≡C—Si(CH₃)₃ and —C≡C—Si(t-Bu)(CH₃)₂ preferred.

“O-linked moiety”, “O-linked group” and like terms as used herein refers to an oxygen-based group or moiety that is attached to an organic moiety, such as an androst-5-ene or 5α-androstane steroid, directly though an oxygen atom of the oxygen-based group or moiety. An O-linked group may be a monovalent O-linked moiety and include moieties such as —OH, an ester, such as acetoxy, i.e., —O—C(O)—CH₃, or acyloxy, i.e., —O—C(O)—R¹², wherein R¹² is —H, optionally substituted alkyl, optionally substituted cycloalkyl, optionally substituted alkenyl, optionally substituted alkynyl, optionally substituted aryl, optionally substituted heteroaryl or optionally substituted heterocycle. Monovalent O-linked moieties further include ether and silyl ether moieties such as alkyloxy, aryloxy (Aryl-O—), phenoxy (Ph-O—), benzyloxy (Bn-O—), heteroaryloxy (Het-O—) and silyloxy, i.e., R¹¹O—, wherein R¹¹ is optionally substituted alkyl, aryl, phenyl, benzyl (—CH₂Ph), heteroaryl or silyl, i.e., (R¹³)₃Si—, wherein R¹³ independently are alkyl or aryl, optionally substituted. Other monovalent O-linked moieties are carbamates having the structure —O—C(O)N(R¹⁴)₂, wherein R¹⁴ independently are —H, optionally substituted alkyl, optionally substituted cycloalkyl, optionally substituted alkenyl, optionally substituted alkynyl or another monovalent C-linked moiety, or carbonates having the structure —O—C(O)OR¹⁵ wherein R¹⁵ is optionally substituted alkyl or another monovalent C-linked moiety, and —OR^{PR}, wherein R^{PR} is a protecting group as previously defined, or an O-linked moiety may be divalent, i.e., ═O or —OCH₂CH₂O—. Preferred monovalent O-linked moieties are esters having the structure —O—C(O)—R¹² and silyl ethers having the structure (R¹³)₃SiO—. For particularly preferred esters, R¹² is C₁₋₆ alkyl or the species —CH₃ (i.e., acetate), —CH₂CH₃ (i.e., propionate), -Ph (i.e., benzoate), —CH₂Ph (phenylacetate) and 4-nitrophenyl (i.e., p-nitrobenzoate) with —CH₃ especially preferred. For particularly preferred silyl ethers (i.e., silyloxy moieties), R¹³ independently are C₁₋₆ alkyl or aryl including —CH₃, —CH₂CH₃, t-butyl or -Ph with trimethylsilyloxy and t-butyldimethylsilyl-oxy moieties especially preferred.

Divalent O-linked moieties include ═O, as when independently in a compound of Formula 1, 2, 3 or 4 both of R¹, R² or R³ together are ═O or if one R¹⁰ replacing two hydrogens at position C-1, C-2, C-11 or C-15 is ═O or are moieties that comprise a cyclic ketal or cyclic thioketal of the aforementioned ═O moiety.

Typically, cyclic ketals and cyclic thioketals comprise an optionally substituted alkyl moiety containing about 2-20 carbon atoms, typically 2 to 3, that connect the two heteroatoms of the ketal or thioketal, and a carbon of another organic moiety, such as the C-17 or C-7 carbon of an androst-5-ene or 5α-androstane steroid nucleus, to which the heteroatoms are attached whereby a spiro ring system is defined. Typically, the alkyl moiety is an C₂₋₆ alkylene (i.e., —(CH₂)₂₋₆— optionally substituted or a branched alkyl, including structures such as —CH₂C(CH₃)₂—, —CH₂CH(CH₃)—, —CH₂—CH₂—, —[CH₂]_{2,3}—, —CH₂—[C(C₁₋₄ alkyl)₂]_{1,2,3}—, —CH(C₁₋₄ alkyl)-[CH(C₁₋₄ alkyl)]_{1,2,3}- or —C(C₁₋₄ alkyl)-2-[CH(C₁₋₄ alkyl)]_{1,2,3}—, wherein C₁₋₄ alkyl are independently selected. Divalent O-linked moieties that comprise a cyclic ketal or cyclic thioketal typically have the structure —X—C(R¹⁶)₂—C(R¹⁶)₂—Y—, wherein —C(R¹⁶)₂—C(R¹⁶)₂— is the optionally substituted C₂₋₆ alkylene, previously defined, and R¹⁶ independently are —H or C₁₋₄ alkyl or two of R¹⁶ and the carbon(s) to which they are attached comprise a cycloalkyl moiety and the other R¹⁶ independently are —H or C₁₋₄ alkyl or two of R¹⁶ together form an o-catechol, where the remaining R¹⁶ are replaced by a double bond, and X and Y independently are O or S. For certain cyclic ketals, the steroid nucleus carbon is bonded through the two oxygen atoms of a divalent O-linked moiety having the structure —O—C(R¹⁶)₂—C(R¹⁶)₂—O— with R¹⁶ as previously defined. For certain cyclic thioketals, a steroid nucleus carbon is bonded through one oxygen and one sulfur atom or, more often, through two sulfur atoms of a divalent O-linked moiety, having the structure —O—C(R¹⁶)₂—C(R¹⁶)₂—S— or —S—C(R¹⁶)₂—C(R¹⁶)₂—S— with R¹⁶ as previously defined.

Ketal moieties, such as cyclic ketals moieties, may serve as protecting groups for a ketone, which can be removed by chemical synthesis methods, with preferred cyclic ketals having divalent O-linked moieties with the structure of —O—CH₂—CH₂—CH₂—O— or —O—CH₂—CH₂—O— that form a spiro ring (i.e., a cyclic ketal) with the carbon to which the heteroatoms of this divalent moiety are attached. For any Spiro ring disclosed herein and unless otherwise specified, the 1^{st} and 2^{nd} open valences can be bonded to the carbon in the steroid nucleus in the α- and β-configurations. For example, in cyclic thioketals having the —S—CH₂—CH₂—O— structure, the 1^{st} open valence, i.e., at the sulfur atom, can be, e.g., at the C-17 position in the β-configuration and the 2^{nd} open valence, i.e., at the oxygen, would then be in the α-configuration or visa versa.

“C-linked moiety”, “C-linked group” and like terms as used herein refers to a moiety or group that is attached to another organic moiety, such as an androst-5-ene or 5α-androstane steroid, directly though a carbon atom of the C-linked moiety or group. An C-linked moiety may be monovalent, including groups such as acyl, i.e., —C(O)—R¹², wherein R is —H, optionally substituted alkyl, optionally substituted cycloalkyl, optionally substituted alkenyl, optionally substituted alkynyl, optionally substituted aryl, optionally substituted heteroaryl or optionally substituted C-heterocycle or carboxylate, i.e., —C(O)—OR¹², wherein R¹² is —H or its corresponding salt, —C(O)—O⁻, or is as previously defined for ester wherein R¹² includes alkyl, aryl, a C-bonded heteroaryl or a C-bonded heterocycle or may be divalent, i.e., ═C(R¹⁰)₂, wherein R¹⁰ independently are —H, aryl, heterocycle, heteroaryl, optionally substituted alkyl, optionally substituted cycloalkyl, optionally substituted alkenyl, optionally substituted alkynyl or a monovalent O-linked moiety including —OH, —OR^{PR}, an O-linked ester, an ether, a carbonate and an O-linked carbamate. Preferred C-linked moieties are C₁₋₄ alkyl, C₂₋₄ alkenyl or C₂₋₄ alkynyl with —CH₃, —CH₂═CH₂, and —C≡CH particularly preferred. Acyl is specifically excluded from “monovalent C-linked moiety”, unless suitably protected, when this substituent results in a 3β-hydroxy-androst-5-ene, 3β-hydroxy-androst-5-ene-7-one, 3α,4α-epoxy-androst-5-en-7-one or 3,5-androst-3,5-diene precursor or intermediate steroid with acyl at position C-2, C-3 or C-17. In such instances the term “monovalent C-linked moiety” is to be understood as a monovalent C-linked moiety that is defined herein other than acyl.

“Steroid” as used here means a substance within a class of compounds that share a similar chemical backbone of 17 carbon atoms contained within the four rings

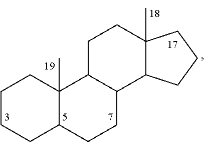

which are common to compounds such as estrogen, testosterone, cortisone and cholesterol. Androst-5-enes and 5α-androstanes with no C-linked moiety at the 17-position are examples of C19-steroids, since their base structure contains 19 carbon atoms while estrenes and 5α-estranes with no C-linked moiety at the 17-position are examples of C18-steroids, since their base structure lacks carbon at the 19-position and thus contains 18 carbon atoms.

“Hydroxy steroid” as used here means a steroid having a substituent at position C-3 that is hydroxy or is a monovalent O-linked moiety that is convertible in a subject to hydroxy and having another substituent at position C-3 that is —H or a C-linked moiety, such as optionally substituted alkyl. A 3β-hydroxy steroid is a hydroxy steroid having hydroxy or a monovalent O-linked moiety that is convertible in a subject to hydroxy at position C-3 in the β-configuration and having the other C-3 substituent as —H or a C-linked moiety, such as optionally substituted alkyl, in the α-configuration. A 3α-hydroxy steroid is a hydroxy steroid having hydroxy or a monovalent O-linked moiety that is convertible in a subject to hydroxy at the C-3 position in the α-configuration and having another substituent at position C-3 that is —H or a monovalent C-linked moiety, such as optionally substituted alkyl. A 3α-O-linked steroid as used here means a steroid having a monovalent O-linked moiety as defined herein, including hydroxy, ester, ether, silyl ether, carbonate or carbamate, where the monovalent O-linked moiety is at position C-3 of the steroid in the α-configuration and is covalently bonded to the steroid through the oxygen atom of the monovalent O-linked moiety, and having the other substituent at position C-3 in the β-configuration as —H or a monovalent C-linked moiety such as optionally substituted alkyl. A 3β-O-linked steroid as used here means a steroid having a monovalent O-linked moiety as defined herein, including hydroxy, ester, ether, silyl ether, carbonate or carbamate, where the monovalent O-linked moiety is at position C-3 of the steroid in the β-configuration and is covalently bonded to the steroid through the oxygen atom of the monovalent O-linked moiety, and having the other substituent at position C-3 in the α-configuration as —H or a monovalent C-linked moiety such as optionally substituted alkyl.

“Substantially free” as used herein refers to a preparation of a compound wherein more than about 80% by weight of the preparation's product is the specified compound. Typically compound the preparation is obtained by the methods described herein. The term “3α-O-linked steroid product substantially free of 3β-O-linked steroid product” refers to a synthetic preparation of a 3α-O-linked steroid wherein more than about 80% of the steroid product is the desired 3α-O-linked steroid, i.e., no more than about 20% of the total hydroxysteroid product may be present as the 3β-O-linked steroid). The term “3α-O-linked androst-5-ene product substantially free of 3α,5α-cycloandrostane” refers to a synthetic preparation of a 3α-O-linked androst-5-ene steroid wherein more than about 80% of the steroid product is the 3α-O-linked androst-5-ene steroid, i.e., no more than about 20% of the total steroid product may be present as 3α,5α-cycloandrostane steroid side-product(s). Such compositions typically contain at least about 95%, preferably at least about 99%, of the desired 3α-O-linked steroid with the remaining steroid present as the so defined by-product, side product, contaminant or other process impurity.

“Essentially free” as used herein refers to a property of or an impurity in a preparation of an F1C as not being present or measurable in an amount that would adversely affect or detract from the desired biological activity or acceptability of the 3α-hydroxy steroid or a 3α-O-linked steroid. For example, the term “essentially free of 3β-hydroxy steroid impurity” refers to the absence or an amount of 3β-hydroxy steroid process impurity(ies) in a preparation of a 3α-hydroxy steroid or a 3α-O-linked steroid that would not adversely affect the biological activity or pharmaceutical acceptability of the 3α-hydroxy steroid or 3α-O-linked steroid for its intended use by contributing undesired biological effects normally attributable to 3β-hydroxy steroids.

“Substantially pure” as used herein refers to a 3α-hydroxy steroid or a 3α-O-linked steroid, such as an androst-5-ene or a 5α-androstane steroid that contain less than about 3% by weight, preferably less than about 2% by weight, total impurities, including residual solvent or process impurities, such as steroid impurities, or more preferably less than about 1% by weight water or residual organic solvent (not inclusive of a desired hydrate or solvate) and/or less than about 0.5% by weight steroid impurities such as decomposition products, synthesis by-products or side products or other steroid process impurities.

The terms “steroid impurity” or “steroid process impurity” as used herein refers to a steroid component in a preparation of an 3α-hydroxy steroid or a 3α-O-linked steroid that is a contaminant, byproduct, side product, degradation product or other steroid process impurity that is formed from or is present in a 3β-hydroxy steroid precursor that is carried through a synthesis of a 3α-hydroxy steroid or a 3α-O-linked steroid, and represents a minority contribution to the overall mass of the steroid preparation. Steroid impurities in a preparation of a 3α-hydroxy steroid product or a 3α-O-linked steroid derived from the 3α-hydroxy steroid product include 3β-hydroxy steroids, other 3β-O-linked steroids derived therefrom and 3α,5α-cycloandrostanes.

“Epoxidizing agent” as used herein refers to a reagent capable alone or in conjunction with other agents of donating an oxygen atom to an alkene to form the corresponding epoxide. Epoxidizing agents suitable for use in the methods described herein include peroxides such as H₂O₂, NaOCl and alkyl hydroperoxides, e.g., t-butylhydroperoxide and cumene hydroperoxide, with or without a transition metal catalyst, singlet O₂, dioxiranes, e.g., dimethyldioxirane, peracids, e.g., performic acid, peracetic acid, perbenzoic acid and m-chloroperbenzoic acid and peracids formed in situ with acid anhydrides and a peroxide. Preferred epoxidizing agents are peracids with m-chloroperbenzoic acid particularly preferred.

“Hindered base” as used herein refers to a nitrogen containing compound wherein the nitrogen is not capable or poorly capable of participating in nucleophillic displacement reactions under reaction conditions of its intended use and is capable of extracting a proton from a carboxylic acid to form the corresponding carboxylate anion to a substantially complete extent at concentrations of the hindered base typically used for chemical transformations described herein. Typically, the nitrogen containing compound will have the structure (R¹⁷)₃N wherein R¹⁷ are independently selected C₁₋₆ alkyl or is a nitrogen containing heterocycle wherein one or more, typically one or two, nitrogens comprise a bicyclic ring system with the nitrogens in bridgehead positions. Typically, the conjugate acid of the nitrogen in the hindered base will have a pKa of about 7 or more, typically between about 7-14, more typically between about 7-12. Preferred hindered bases include N-methylmorpholine, N-methylpiperidine, triethylamine and N,N′N″-diisopropylethyl amine (Hunig's base).

“Tri-substituted phosphine” as used herein refers to a phosphorous containing compound to which is covalently attached three monovalent C-linked moieties such that the phosphorous is nucleophillic and is capable of forming a nitrogen based anion upon its interaction with an azo-di-carboxylate ester under reaction conditions typically employed for the Mitsunobu reaction. Typically, the tri-substituted phosphine will have the structure (R¹⁸)₃P wherein R¹⁸ independently selected are C₁₋₆ alkyl or aryl. Preferred tri-substituted phosphines include tributylphosphine and triphenylphosphine.

“Organic acid” as used herein is a compound having the structure of R¹²C(O)OH wherein R¹² is a monovalent C-linked moiety, such as optionally substituted alkyl or optionally substituted aryl. Organic acids include acetic acid, benzoic acid and other aryl organic acids, i.e., organic acids having the structure (optionally substituted) ArCO₂H where the aryl group is unsubstituted or substituted with one or more, typically one or two, electron withdrawing groups such as halogen or —NO₂, with p-NO₂-benzoic acid (i.e., R¹² is 4-nitrophenyl) preferred.

“Azo-di-carboxylate ester” as used herein is a compound having the structure of R¹⁹OC(O)N═NC(O)OR¹⁹ wherein R¹⁹ are independently selected alkyl, typically C₁₋₆ alkyl. Preferred azo-di-carboxylate esters have R¹⁹ as C₁₋₄ alkyl and include species diethyl azodicarboxylate (DEAD) where R¹⁹ is —CH₂CH₃ or di-isopropyl azodicarboxylate (DIAD) where R¹⁹ is —CH(CH₃)₂.

Polar non-protic solvent as used herein is a solvent capable of stabilizing charge-separated reaction intermediates by non-hydrogen bonding interactions and include ethers such as ethyl ether, tetrahydrofuran and dioxane and N-substituted amides such as N,N′ dimethylformamide (DMF) and N-methylpyrrolidinone (NMP).

“Hydride donor” as used herein is a reducing agent or reducing reaction conditions that reduces a divalent O-linked moiety to a monovalent O-linked moiety by transfer of a hydride atom to the carbon atom to which the divalent O-linked moiety is bonded or is a reducing agent or reducing reaction conditions that transfers a hydride atom to an epoxide to result in reductive epoxide opening. For reactions described herein that use a hydride donor, a carbinol typically is formed from reduction of the divalent O-linked substituent ═O (ketone) or an epoxide after quenching such reactions with a proton donor. Other monovalent O-linked substituents may be formed by quenching the initially formed carbinol anion with electrophiles (e.g., formation of acetate after quenching with an acetyl halide, such as acetyl chloride, or a methyl ether after quenching with methyl iodide).

Hydride donors include hydrides of aluminum including LiAlH₄ (LAH), alkyl aluminum hydrides such as di-isobutyl aluminum hydride (DIBAL-H) and tri-butyl aluminum hydride and alkoxy aluminum hydrides such as sodium bis(2-methoxyethoxy)aluminium hydride (Red-Al), lithium trimethoxyaluminum hydride (LTMA) and (lithium triethoxyaluminum hydride) (LTEAH). Preparation and use of alkoxy aluminum hydrides as hydride donors is given in U.S. Pat. No. 3,281,443 (specifically incorporated by reference herein). Preparation and use of di-alkyl and tri-alkyl aluminum hydrides as hydride donors is given in Ziegler, K., et al. “Metallorganische Verbindungen, XXVII Aluminiumtrialkyle und Dialkyl-Aluminiumhydride Aus Aluminiumisobutyl-Verbindungen”. Justus Liebig's Annalen der Chemie 629 (1): 14-19 (1960).

Hydride donors further include hydrides of boron including NaBH₄, KBH₄, LiBH₄, and alkyl borohydrides such as lithium tri-sec-butylborohydride (L-Selectride), potassium tri-sec-butylborohydride (K-Selectride), and lithium n-butylborohydride. Preparation and use of simple boron hydrides and trialkylborohydrides is given in Walker, E. R. H. “The functional group selectivity of complex hydride reducing agents” Chem. Soc. Rev. 5:23-50 (1976); Brown H. C., et al. Tet. 35: 567 (1979). Preparation and use of mono- and di-alkyl-borohydrides is given in Brown, H. C., et al. “Addition compounds of alkali metal hydrides. 20. Reaction of representative mono- and dialkylboranes with saline hydrides to form the corresponding alkylborohydrides” J. Org. Chem. 46:2712-2717 (1981). Other hydride-based reducing systems for ketone reduction include aluminumisopropylate in isopropanol (Meerwein-Ponndorf-Verley Reduction).

Alkylaluminum hydrides and alkylborohydrides having bulky alkyl groups such as isobutyl or sec-butyl will preferentially approach the less hindered face of a steroid nucleus, which is typically the α-face, to provide, optionally after electrophile quenching, a monovalent O-linked substituent in the β-configuration. Alkoxy aluminum hydrides having lowered reactivity and increased steric bulk compared to LAH will provide greater selectivity for the less hindered α-face. Hydride donors also include boron hydride-based reducing systems, e.g., NaBH₄, with a transition metal halide, such as CeCl₂, which polarizes the divalent O-linked substituent ═O to increase its susceptibility to reduction, and will provide predominately a monovalent O-linked substituent in the 6-configuration when the ketone substituent is at position C-7 or C-17. Epoxide reduction (e.g., reduction of steroid 3α,4α-epoxy functional group) with a hydride donor typically requires the more reactive aluminum-based hydrides (in comparison to the boron-based hydrides), with the more reactive LAH hydride donor preferred. Without being bound by theory, contacting a 3α,4α-epoxy-androst-5-en-7-one steroid with LAH is expected to reduce the C-7 ketone predominately from the less hindered α-face of the steroid with subsequent epoxide ring opening through hydride delivery to C-4 to result in a 3α-hydroxy-androst-5-en-7β-ol steroid. Preferred hydride donors for ketone reduction are boron-based hydrides, optionally in the presence of a transition metal halide. Particularly preferred hydride donors are NaBH₄ or NaBH₄ in the presence of CeCl₃.

“Hydrogen atom donor” as used herein refers to a reducing agent or reaction conditions that adds one or more hydrogen atoms other than a hydride to a functional group upon which it acts. Hydrogen atom donors include hydrogen atom-based donor systems, e.g., platinum or palladium metals, or their metal salts or oxides, optionally on a solid support, such as carbon black or calcium carbonate, in the presence of hydrogen gas at hydrogen gas pressures of between about ambient pressure to about 50 psi at or near ambient temperature or at elevated temperature, wherein the elevated temperature is below the boiling point of the solvent system in which the Pd or Pt metal is present. Preferred temperatures for Pd or Pt-based hydrogen atom donor systems are between about ambient to about 40° C. or between about 22° C. to about 40° C., with about 40° C. preferred if elevated temperatures are required as, for example, when the rate of hydrogenation or the solubility of the hydrogen atom acceptor (e.g., an androst-5-ene steroid) is insufficient. Hydrogen atom-based donor systems also include systems that produce hydrogen radicals as the reducing agent as, for example, a tri-alkyl tin hydride such as Bu₃Sn—H in the presence of a free radical initiator or systems involving electrons as the reducing agent as, for example, dissolving metal reductions. Hydrogen donors (i.e., reducing agents) include hydrogen atom donors and hydride donors.

“Eliminating agent” as used herein refers to an agent or reaction conditions capable of removing a monovalent O-linked substituent by elimination, thus forming a double bond between the carbon to which the monovalent O-linked substituent was attached and a directly adjacent carbon atom, which may subsequently migrate under conditions of the elimination. The eliminating agent may be a hindered base, (i.e. basic conditions) that removes a hydrogen from a position adjacent to a monovalent O-linked moiety susceptible to elimination or a Lewis or Brønsted acid (i.e., acidic conditions) that increases the susceptibility of a monovalent O-linked moiety to elimination. Typically, the Brønsted acid will be an organic sulfonic acid in non-aqueous solution. Organic sulfonic acids have the structure R¹²—S(O)₂OH, wherein R¹² is as defined for organic acid, and include alkyl- and arenesulfonic acids such as methanesulfonic acid, benzene-sulfonic acid or p-toluene-sulfonic acid (p-TSA)

“Leaving group” as used herein refers to a substituent of a carbon in the steroid nucleus that is capable of departure and as a result is replaced with another substituent (i.e., nucleophillic displacement) or forms a double bond between the carbon to which the leaving group was attached and an adjacent carbon (i.e., elimination). Typically, the leaving group will be electronegative with respect to the carbon to which it is attached. Oftentimes, use of an eliminating agent, such as a hindered base, will favor elimination of the leaving group over its displacement through abstraction of a proton on the adjacent carbon. When the proton to be abstracted is adjacent to a double bond carbon and to the carbon bearing the electronegative substituent, then elimination, which provides for an extended conjugated system, may be effected using an eliminating agent that is not a hindered base as, for example, in the transformation of a 3β-O-acyloxy-androst-5-dien-7-one steroid to an androst-3,5-dien-7-one steroid, where elimination of a 3β-O-acyloxy is effected under acidic conditions as described for 17,17-ethylenedioxy-3β-O-acetoxy-androst-5-dien-7-one in the preparation of 17,17-ethylenedioxy-3β-O-acetoxy-androst-3,5-dien-7-one (vide infra).

“Suitably protected”, “suitable monovalent O-linked moiety”, “suitable ester and like phrases refers to R¹, R², R³, R⁵, R⁶ and R¹⁰ substituents of steroids having structures defined herein that are selected based upon their ability to resist premature loss, undesired transformation to another substituent, or interfering with a desired chemical transformation under reaction conditions normally employed for the chemical transformation in which the steroid so protected is used as a reactant. For example, a suitably protected 3β-hydroxy-androst-5-ene steroid reactant in the chemical transformation of Method B would have hydroxy substituents that are present other than the 3β-hydroxy substituent protected as, for example, an ester, an ether or silyl ether to avoid interference from these other hydroxy groups with the Mitsunobu reaction. In another example, a suitably protected androst-3,5-diene reactant for the preparation of a 3α,4α-epoxy-androst-5-ene would have ketone substituents that are present would be protected, for example, as a ketal to avoid these substituents from reacting with the epoxidizing agent such that an undesired Bayer-Villiger reaction occurs. Preferably, protecting groups that are to be present in the 3α,4α-epoxy-androst-5-ene, i.e. will not likely interfere with reductive epoxide opening of Method A from contact of the 3α,4α-epoxy steroid with a hydrogen donor, would also be present in the androst-3,5-diene steroid precursor and thus these protecting groups should be resistant to epoxidizing and reducing agents required to effect the desired chemical transformations so that protecting group manipulations are minimized. In yet another example a suitable hydroxy protecting group for reactions employing organometallic reagents such as addition of an organometallic reagent to a carbonyl of a steroid reactant (e.g., C-17 ═O of an androst-5-en-17-one or an 5a-androstan-17-one) to from a substituted carbinol is a silyl ether having the structure of (R¹³)₃SiO— with R¹³ as defined herein.

“Formulation” or “pharmaceutically acceptable formulation” as used herein refers to a composition comprising a preparation a 3α-hydroxy steroid or 3α-O-linked steroid and one or more pharmaceutically acceptable excipients.

An “excipient” as used herein means a component or an ingredient, other than the active pharmaceutical ingredient, that is included in a invention composition or formulation and has been found acceptable in the sense of being compatible with the other ingredients of invention compositions or formulations. Excipients typically used in the pharmaceutical formulation arts include one or more diluents, disintegrants, binders, anti-adherents, lubricants, glidants, sorbents, suspension agents, dispersion agents, wetting agents, surface-active agents, flocculating agents, buffering agents, tonicity-adjusting agents, metal chelator agents, anti-oxidants, preservatives, fillers, flow enhancers, compression aids, colors, sweeteners, film formers, film coatings or flavoring agents.

“Pharmaceutically acceptable” as used herein in reference to the different composition or formulation components, or the composition or formulation itself, means that the components of the composition or formulation itself do not cause unacceptable adverse side effects in relation to the condition and the subject being treated. Examples of pharmaceutically acceptable components are provided in United States Pharmacopoeia and National Formulary, USP 30-NF 25, May 2007 (specifically incorporated by reference herein).

Invention embodiments provide reaction methods or sequences for preparing a formula 1 compound (F1C) wherein the F1C has the structure

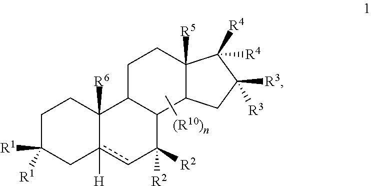

wherein R¹ in the α-configuration is a monovalent O-linked moiety, such as —OH, —OR^{PR}, an ester, an ether or a silyl ether, and R¹ in the β-configuration is —H or an optionally substituted alkyl; R² independently are —H, a monovalent O-linked moiety such as —OH, —OR^{PR}, an ester, an ether or a silyl ether or a monovalent C-linked moiety, such as optionally substituted alkyl, optionally substituted alkenyl or optionally substituted alkynyl; R³ independently are —H, halogen, a monovalent O-linked moiety such as —OH, —OR^{PR}, an ester, an ether or a silyl ether, or a monovalent C-linked moiety, such as optionally substituted alkyl, optionally substituted alkenyl or optionally substituted alkynyl; one R⁴ is in the β-configuration and is a monovalent O-linked moiety such as —OH, —OR^{PR}, an ester, an ether or a silyl ether and the other R⁴ in the α-configuration is —H or a monovalent C-linked moiety (in some embodiments this configuration at C!7 is inverted), such as optionally substituted alkyl, optionally substituted alkenyl or optionally substituted alkynyl, or independently both R², R³ or R⁴ together are a divalent O-linked moiety such as ═O (ketone) or —XC(R¹⁶)₂C(R¹⁶)₂Y—, which defines a spiro ketal or thioketal ring system (i.e., —XC(R¹⁶)₂C(R¹⁶)₂Y— comprises a cyclic ketal or cyclic thioketal), wherein the divalent O-linked moiety has the structure wherein X, Y and R¹⁶ are as defined for cyclic ketal or cyclic thioketal; R⁵ and R⁶ independently are —H or a monovalent C-linked moiety such as optionally substituted alkyl, optionally substituted alkenyl or optionally substituted alkynyl; wherein (R¹⁰)ₙ is 0, 1, 2, 3 or 4 independently selected R¹⁰ substituents (i.e., n=0 to 4) attached to the steroid ring replacing hydrogen other than at positions C-3, C-7, C-16 and C-17, preferably at none, one, two, three or four positions selected from the group consisting of positions C-1, C-2, C-4, C-6, C-9, C-11, C-12 and C-15, wherein none, one or two R¹⁰ may be present at positions C-1, C-2, C-11 and C-15 and none or one R¹⁰ may be present at positions C-4, C-6 or C-9, wherein R¹⁰, if present at position C-9 is —Cl or —F, if present at positions C-4 or C-6 is independently selected optionally substituted alkyl and if present at positions C-1, C-2, C-11 or C-15 is independently selected halogen, a monovalent C-linked moiety, such as an optionally substituted alkyl, a monovalent O-linked moiety, such as —OH, —OR^{PR}, ester, ether or silyl ether or a divalent O-linked moiety such as ═O or —XC(R¹⁶)₂C(R¹⁶)₂Y— where X, Y are attached to the same carbon of the steroid ring system, wherein R¹⁶ are as defined for cyclic ketal or cyclic thioketal; and —H at position C-5, if present, is in the α configuration.

In preferred embodiments, (a) R⁵ and R⁶ are —CH₃ in the β-configuration or R⁵ is —CH₃ in the β-configuration and R⁶ is —H in the β-configuration or R⁵ is —CH₂OH in the 3-configuration and R⁶ is —CH₃ in the β-configuration and (b) R⁴ in the β-configuration is a —OH or an ester and the other R⁴ in the α-configuration is —H or a monovalent C-linked moiety with optionally substituted alkyl and optionally substituted alkynyl preferred and —CH₃ and —C≡CH particularly preferred. Specifically excluded are structures having a pentavalent carbon (e.g., —H at position C-5 is absent if a double bond is present between positions C₅-C₆).

Ethers, including aliphatic and aromatic ethers, typically have the structure R¹¹O—, wherein R¹¹ is optionally substituted alkyl, including optionally substituted cycloalkyl, optionally substituted aryl or optionally substituted heteroaryl. Typically, esters have the structure R¹²C(O)O— wherein R¹² is —H, optionally substituted alkyl, including optionally substituted cycloalkyl, optionally substituted aryl or optionally substituted heteroaryl with C₁₋₆ alkyl, C₃₋₆ cycloalkyl and optionally substituted phenyl preferred. Typically silyl ethers have the structure (R¹³)₃SiO— wherein R¹³ independently are alkyl or aryl with methyl, ethyl, t-butyl and phenyl preferred.

Large scale manufacturing of such compounds for therapeutic purposes, e.g., in human clinical trial protocols or in large scale preclinical studies, such as long term large animal toxicology studies, are needed to support human clinical protocols. The present disclosure relates to the discovery of improved methods to prepare 3α-hydroxy steroids on a large scale.

The 3α-O-linked steroids have a range of biological activity, e.g., certain 3α-monovalent-O-linked, 17-oxygen substituted (mono or divalent O-linked) steroid compounds having no, one or two O-linked moieties at positions C-7 and C-16 such as androst-5-ene-3α,7β,16α,17β-tetrol 17α-ethynyl-androst-5-ene-3α,7β,17β-triol are useful to treat or ameliorate metabolic disorders such as type 2 diabetes, hyperglycemia, hyperlipidemia or hypercholesterolemia and inflammation and autoimmune conditions such as asthma, chronic obstructive pulmonary disease, chronic bronchitis or arthritis or inflammatory bowel disorders such as ulcerative colitis, while other 3α-oxygen, 17-oxygen substituted steroid compounds such as 17α-ethynyl-5α-androstane-3α,17β-diol, 17α-ethynyl-5α-androstane-2α,3α,17β-triol, 17α-ethynyl-5α-androstane-2β, 3α,17β-diol, 17α-ethynyl-5α-androstane-3α,7α,17β-triol, 17α-ethynyl-5α-androstane-3α,7β,17β-triol and 17α-ethynyl-5α-androstane-3α,16α,17β-triol are useful to treat or ameliorate hyperproliferation conditions such as cancer, a hyperplasia or related conditions, e.g., prostate cancer, breast cancer, lung cancer, colon cancer and benign prostatic hyperplasia. Administration of an effective amount of an aforementioned compound can be used to treat these conditions.

In some reports, 3α-hydroxy-androst-5-ene steroids have been prepared from androst-4-en-3-one steroids by stereoselective ketone reduction or indirectly from 3β-hydroxy-androst-5-enes, through their conversion to androst-4-en-3-one steroids, or from direct inversion of configuration of the 3β-hydroxy substituent. However, these methods would typically provide 3α-hydroxy steroid products having 3β-hydroxy steroid or 3α,5α-cycloandrostane steroid impurities that may be in pharmaceutically unacceptable amounts. Methods relying upon stereoselective reduction of androst-4-en-3-one steroids for establishing the 3α-configuration have required expensive chiral reducing agents and usually subzero reaction temperatures, which add significantly to large scale manufacturing costs. Direct inversion of configuration, e.g., by the Mitsunobu reaction of a 3β-hydroxy steroid precursors or nucleophillic displacement of a reactive monovalent O-linked moiety, such as a sulfonate derived from a 3β-hydroxy steroid, can be impaired by participation of the Δ⁵-double bond. This participation typically leads to loss of stereoselectivity (i.e., 3α-O— linked steroid products with 3β-hydroxy steroid impurities) and formation of 3α,5α-cycloandrostanes side product(s). As a result, the use of such direct inversion methods for research scale production of highly purified end products would require masking of the double bond through di-halogenation or other reversible chemical transformations, which adds additional steps and hence increased cost to the manufacturing process.

For 3α-hydroxyandrostane steroids, methods for small scale (i.e., research-scale) preparation would typically rely upon 5α-androstane precursors already having the 3α-hydroxy substituent. However, methods to prepare 3α-hydroxyandrostane steroids and other androstane steroids having an oxygen substituent in the 3α-configuration from more abundant and less expensive 3β-hydroxyandrost-5-ene steroid precursors would be useful for larger scale preparation, i.e., non-research uses at scales, for example, of 25 g, 100 g or more. Heretofore, we do not believe large scale synthetic methods have previously been needed for such compounds.

The afore-described aspects of preparing 3α-hydroxy-androst-5-ene steroids, 3α-hydroxy-5α-androstane steroids, and related steroids, are addressed by the present methods. Large scale methods to prepare 3α-hydroxysteroids, such as 3α-hydroxyandrost-5-enes, 3α-hydroxyandrostanes and other related steroids having an oxygen substituent in the 3α-configuration with lowered 3β-hydroxy steroid or 3α,5α-cycloandrostane impurity burden have not to our knowledge been described or needed. Such methods, therefore, are useful to provide materials suitable for commercial scale production of 3α-hydroxy-androst-5-ene steroids, 3α-hydroxy-5α-androstane steroids.

One solution to the previously unappreciated need for larger scale synthesis methods for preparing 3α-O-linked steroids provide new 3α,4α-epoxy-androst-5-en-7-one steroids, which are used as synthesis intermediates in Method A described herein. From this method 3α-hydroxyandrost-5-ene steroids, 3α-hydroxyandrostane steroids and related steroids, are prepared more efficiently and economically less costly. Preferred 3α,4α-epoxy-androst-5-en-7-one intermediates for Method A include 17,17-ethylenedioxy-3α,4α-epoxy-androst-5-en-7-one, 17,17-dimethoxy-3α,4α-epoxy-androst-5-en-7-one, 17,17-ethylenedioxy-3α,4α-epoxy-androst-5-en-7-one-16α-ol, 17,17-ethylenedioxy-16α-acetoxy-3α,4α-epoxy-androst-5-en-7-one, 17,17-ethylenedioxy-16α-methoxy-3α,4α-epoxy-androst-5-en-7-one, 17,17-ethylenedioxy-16α-trimethylsilyloxy-3α,4α-epoxy-androst-5-en-7-one, 17,17-ethylenedioxy-16α-methyl-3α,4α-epoxy-androst-5-en-7-one, 17,17-ethylenedioxy-16α-propyl-3α,4α-epoxy-androst-5-en-7-one, 17,17-ethylenedioxy-16α-(prop-2-yl)-3α,4α-epoxy-androst-5-en-7-one and 17,17-ethylenedioxy-16α-(prop-1-yl)-3α,4α-epoxy-androst-5-en-7-one.

Another solution for larger scale synthesis of 3α-O-linked steroids, described herein as Method B, defines Mitsunobu reaction conditions that have been unexpectedly found to be effective for direct stereochemical inversion of 3β-hydroxy-androst-5-ene steroids to provide 3α-hydroxy-androst-5-ene steroids with surprisingly reduced amounts of reaction side-products such as 3α,5α-cycloandrostanes that were previously observed from small scale syntheses. These reduced amounts of reaction side-products is in comparison to reported indirect methods for inversion of configuration of a hydroxy group at position C-3 of an androst-5-ene steroid. These indirect methods typically involve conversion of a 3β-hydroxy group in a 3β-hydroxy-androst-5-ene steroid to a good leaving group, such as an alkyl or arylsulfonate, that is capable of displacement by a O-linked nucleophile [for example, see Neeland, et al. Synth. Comm. 19:13-14 (1989); Ruddock, et al. Steroids 63:650-664 (1998); McCarthy, et al. Org. Bioorg. Chem. 3(16):3059-3065 (2005)].

The reaction sequences disclosed herein further provide efficient synthetic methods that obviate the need for using steroids that have an O-linked oxygen substituent at position C-3 in the β-configuration, which may have undesired biological activity (ies), as precursors or advanced synthetic intermediates in the commercial preparation of steroids having a monovalent O-linked oxygen moiety at position C-3 in the α-configuration. As a consequence steroids having a 3β-O-linked moiety, such as a 3β-hydroxy steroid, with potential undesired biological activity (ies) or in amounts that are pharmaceutically unacceptable are avoided, or less likely carried forward, as impurities in steroid products having a 3α-O-linked moiety with desired biological activity, such as a 3α-hydroxy steroid product. Thus, 3α-O-linked steroids are obtained with reduced undesired biological effects or in pharmaceutically acceptable purity with respect to 3β-hydroxy steroid contaminants or contaminants derived therefrom in comparison to previous methods using 3β-O-linked steroids as precursors or late stage intermediates prepared on research scale.

In view of the forgoing a principal embodiment of the invention provides a reaction sequence for inverting configuration at the C-3 position of a 3β-hydroxy steroid having a Δ⁵-ene double bond that proceeds through a 3α,4α-epoxy-androst-5-en-7-one precursor or intermediate.

Another principal embodiment of the invention provides a reaction sequence for inverting configuration at the C-3 position of a 3β-hydroxy steroid having a Δ⁵-ene that does not require masking of this double bond to mitigate formation of undesired side products such as 3α,5α-cycloandrostanes.

In yet other embodiments of the invention, reaction sequences are provided for preparing 3α-hydroxy-androst-5-ene steroids, including their ester, ether, silyl ether and other monovalent O-linked derivatives, additionally having one or two monovalent O-linked substituent(s) at the C-17 position or a divalent O-linked substituent at the C-17 position, optionally having one or more O-linked substituents, including monovalent and divalent C-linked substituents, at the C-7 or C-16 positions. Such steroids, which themselves may be used as intermediates for the preparation of additional 3α-O-linked-androst-5-ene steroids and 3α-O-linked-5α-androstane steroids, include androst-5-en-7,17-dione-3α-ol, androst-5-ene-3α,7α,17β-triol, androst-5-ene-3α,7β,17β-triol, 3α-acetoxy-androst-5-en-17-one-7β-ol, 3α,7β-di-acetoxy-androst-5-en-17-one, 3α,7β-di-(trimethylsilyloxy)-androst-5-en-17-one, androst-5-ene-3α,7β,16α,17β-tetrol, androst-5-ene-3α,7α,16α,17β-tetrol, 16α-methoxy-androst-5-ene-3α,7β,17β-triol, 16α-methyl-androst-5-ene-3α,7β,17β-triol, 16α-propyl-androst-5-ene-3α,7β,17β-triol and 16α-(prop-2-yl)-androst-5-ene-3α,7β,17β-triol. In another embodiment of the invention, reaction sequences are provided for preparing 3α-hydroxy steroids and 3α-O-linked steroids, including ester, ether, silyl ether, and other monovalent O-linked derivatives, having two substituents at the C-17 position, wherein one substituent is a monovalent O-linked moiety (e.g., is not ═O) and the other substituent is a monovalent C-linked moiety, wherein the monovalent C-linked moiety is, for example, an optionally substituted alkyl group, an optionally substituted alkenyl group or an optionally substituted alkynyl group, and optionally having one or more O-linked moieties, including monovalent and divalent O-linked moieties at the C7- or C-16 positions or the C-7 and C-16 positions. Such steroids include 17α-ethynyl-androst-5-ene-3α,7β,17β-triol, 17α-ethynyl-androst-5-ene-3α,7β,16α,17β-tetrol, 17α-methyl-androst-5-ene-3α,7β,17β-triol, 17α-ethynyl-androst-5-ene-3α,17β-diol, 17α-ethenyl-androst-5-ene-3α,7β,17β-triol and 17α-(propyn-3-ol-1-yl)-androst-5-ene-3α,7β,17β-triol.

Another embodiment of the invention provides reaction sequences for preparing 3α-hydroxy-5α-androstane steroids, including their ester, ether, silyl ether and other monovalent O-linked derivatives from 3α-hydroxy-androst-5-ene steroid intermediates, which are prepared according to methods described herein, additionally having one or two monovalent O-linked substituent(s) at the C-17 position or a divalent O-linked substituent at the C-17 position, optionally having one or more O-linked substituents, including monovalent and divalent O-linked substituents, at the C-7 or C-16 positions. Such steroids, which themselves may be used as intermediates for the preparation of additional 3α-O-linked-5α-androstane steroids, include 5α-androstan-7,17-dione-3α-ol, 5α-androstane-3α,7α,17β-triol, 5α-androstane-3α,7β,17β-triol, 5α-androstane-3α,16α,17β-triol, 3α-acetoxy-5α-androstan-17-one-76-ol, 3α,76-di-acetoxy-5α-androstan-17-one, 3α,76-di-(trimethylsilyloxy)-5α-androstan-17-one, 5α-androstane-3α,76,16α,17β-tetrol, 5α-androstane-3α,7α,16α,17β-tetrol, 16α-methoxy-5α-androstane-3α,7β,17β-triol, 16α-methyl-5α-androstane-3α,7β,17β-triol, 16α-propyl-5α-androstane-3α,7β,17β-triol and 16α-(prop-2-yl)-androstane-3α,7β,17β-triol. In another embodiment of the invention, reaction sequences are provided for preparing 3α-hydroxy steroids and 3α-O-linked steroids, including ester, ether, silyl ether, and other monovalent O-linked derivatives, having two substituents at the C-17 position, wherein one substituent is a monovalent O-linked moiety (e.g., is not ═O) and the other substituent is a monovalent C-linked moiety, wherein the monovalent C-linked moiety is, for example, an optionally substituted alkyl group, an optionally substituted alkenyl group or an optionally substituted alkynyl group, and optionally having one or more O-linked moieties, including monovalent and divalent O-linked moieties at the C7- or C-16 positions or the C-7 and C-16 positions. Such steroids include 17α-ethynyl-5α-androstane-3α,7β,17β-triol, 17α-ethynyl-5α-androstane-3α,16α,17β-trio-1,17α-ethynyl-5α-androst-5-ene-3α,76,16α,17β-tetrol, 17α-methyl-5α-androst-5-ene-3α,7β,17β-triol, 17α-ethynyl-5α-androst-5-ene-3α,17β-diol, 17α-ethenyl-5α-androst-5-ene-3α,7β,17β-triol and 17α-(propyn-3-ol-1-yl)-5α-androst-5-ene-3α,7β,17β-triol.

Another embodiment of the invention provides reaction sequences for preparing 3α-hydroxy-androst-5-en-7,17-dione and other 3α-O-linked steroids derived therefrom.

In other embodiments of the invention, reaction sequences for preparing 3α-DHEA and other 3α-O-linked androst-5-ene steroids derived therefrom are provided.

Other embodiments of the inventions provide reaction sequences for preparation of 3α-hydroxy-5α-androstanes and other 3α-O-linked 5α-androstane steroids by way of 3α-O-linked-androst-5-enes prepared from 36-hydroxy-androst-5-enes using the methods disclosed herein.

In some specific embodiments, the invention provides methods or reaction sequences to make 3α-O-linked steroids disubstituted at position 17 or additional oxygen functionality, preferably —OH or an ester such as acetate, at positions C-7 or C-16 or positions C-7 and C-16. These 3α-O-linked steroids include androst-5-ene and 5α-androstane steroids such as 17α-ethynyl-androst-5-ene-3α,7β,17β-triol, 17α-ethynyl-androst-5-ene-3α,7α,17β-triol, 17α-ethynyl-androst-5-en-7-one-3α,17β-diol, 17α-ethynyl-androst-5-ene-3α,76,16α,17β-tetrol, 17α-ethynyl-5α-androstane-3α,7β,17β-triol, 17α-ethynyl-5α-androstane-3α,7α,17β-triol, 17α-ethynyl-5α-androstan-7-one-3α,17β-diol 17α-ethynyl-5α-androst-5-ene-3α,76,16α,17β-tetrol, androst-5-ene-3α,76,16α,17β-tetrol, 5α-androstane-3α,76,16α,17β-tetrol, 17α-ethynyl-5α-androstane-3α,17β-diol and 17α-ethynyl-5α-androstane-2α,3α,17β-triol.

The presently disclosed methods can be used to make 3α-hydroxy-androst-5-ene steroids, 3α-hydroxy-5α-androstane steroids and related steroids from 36-hydroxyandrost-5-enes as disclosed herein. Preferred 3α-hydroxy-androst-5-ene steroids that can be prepared are 3α-hydroxy-androst-5-en-17-one (3α-DHEA), 3α-hydroxy-androst-5-en-7,17-dione, androst-5-ene-3α,76,16α,17β-tetrol, 17α-ethynyl-androst-5-ene-3α,7β,17β-triol and 17α-ethynyl-androst-5-ene-3α,76,16α,17β-tetrol. Preferred 3α-hydroxy-5α-androstane steroids that can be prepared are 17α-ethynyl-5α-androstane-3α,17β-diol, 17α-ethynyl-5α-androstane-2α,3α,17β-triol and 17α-ethynyl-5α-androstane-2α,3α,17β-triol.

The invention methods are suitable to make 3α-hydroxy-5α-androstane and related steroids from 36-hydroxyandrost-5-enes as disclosed herein. Preferred compounds that can be prepared are 5α-androstan-17-one-3α-ol, 5α-androstan-7,17-dione-3α-ol, 5α-androstane-3α,76,16α,17β-tetrol, 17α-ethynyl-5α-androstane-3α,7β,17β-triol and 17α-ethynyl-5α-androstane-3α,76,16α,17β-tetrol.

In one preferred embodiment of the invention, reaction sequences are provided for preparing 3α-hydroxy-androst-5-en-7,17-dione and analogs derived therefrom.

In another preferred embodiments of the invention, reaction sequences for preparing 3α-DHEA and analogs derived therefrom are provided.

Some invention embodiments described herein provide for methods of preparing 3α-hydroxy steroids essentially free of 36-hydroxy steroid impurities and having O-linked substituents at the C-7 and C-17 positions and optionally with additional O-linked substituents at the C-16 position.

Some invention embodiments described herein provide for methods of preparing C17-disubstituted steroids having monovalent O-linked substituents at positions C-3α and C-17β, optionally having an O-linked substituent at C-7 or C-7α/β, that are essentially free of 3β-hydroxy steroid impurities or 3α,5α-cycloandrostane impurities.

Still other invention embodiments described herein provide for methods of preparing 3α-hydroxy steroids essentially free of 3α,5α-cycloandrostane impurities and having O-linked substituents at the C-17 positions and optionally with additional O-linked substituents at the C-7 or C-16 positions.

In some embodiments a 3α-hydroxy steroid is prepared using a reaction sequence comprising the steps of (1) Contacting a suitably protected androst-3,5-dien-7-one steroid with an epoxidizing agent, optionally m-chloroperbenzoic acid (MCPBA), wherein the androst-3,5-dien-7-one steroid has the structure of Formula 2

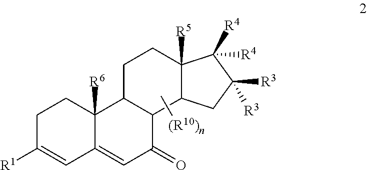

to form an 3α,4α-epoxy-androst-5-en-7-one steroid product of Formula 3; and (2) contacting a suitably protected 3α,4α-epoxy-androst-5-ene steroid of Formula 3

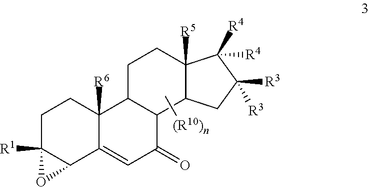

obtained or derived from step 1 with a hydrogen donor, wherein the hydrogen donor is a hydrogen hydride or hydrogen atom donor, optionally lithium aluminum hydride or palladium on charcoal, wherein the 3α,4α-epoxy functional group is preferentially reduced relative to the Δ⁵ functional group with or without concomitant reduction of a C-7 ketone moiety and wherein reduction of the 3α,4α-epoxy functional group occurs preferentially at position C-4 with retention of configuration at position C-3; and wherein in Formula 2 and Formula 3, R¹ is —H or a suitable optionally substituted alkyl; R³ independently are —H, a suitable halogen, a suitable monovalent O-linked moiety, including, e.g., a suitable —OR^{PR}, ester, ether or silyl ether, or a suitable monovalent C-linked moiety, wherein the monovalent C-linked moiety is, for example, a suitable optionally substituted alkyl group; R⁴ independently are a suitable monovalent O-linked moiety, including, e.g., a suitable —OR^{PR}, ester, ether or silyl ether or both of R⁴ together are ═O or define a spiro ketal wherein the spiro ketal comprises the structure —XC(R¹⁶)₂C(R¹⁶)₂Y— or —XC(R¹⁶⁾₂C(R¹⁶)₂C(R¹⁶)₂Y—, wherein X and Y are O and R¹⁶ are as defined for cyclic ketal; R⁵ and R⁶ independently are —H or a suitable optionally substituted alkyl; (R¹⁰)ₙ is 0, 1, 2, 3 or 4 independently selected R¹⁰ substituents (i.e., n=0, 1, 2, 3 or 4) attached to the steroid ring replacing hydrogen other than at positions C-3, C-7, C-16 and C-17, preferably at none, one, two, three or four positions selected from the group consisting of positions C-1, C-2, C-4, C-6, C-9, C-11, C-12 and C-15, wherein none, one or two R¹⁰ may be present at positions C-1, C-2, C-11 and C-15 and none or one R¹⁰ may be present at positions C-4, C-6 or C-9, wherein R¹⁰, if present at position C-9 is —Cl or —F, if present at positions C-4 or C-6 is independently selected optionally substituted alkyl and if present at positions C-1, C-2, C-11 or C-15 is independently selected halogen, a monovalent C-linked moiety, such as an optionally substituted alkyl, a monovalent O-linked moiety, such as —OH, —OR^{PR}, ester, ether or silyl ether or a divalent O-linked moiety such as ═O, —XC(R¹⁶)₂C(R¹⁶)₂Y— or XC(R¹⁶)₂C(R¹⁶)₂C(R¹⁶)₂Y—)₂Y— where X, Y are attached to the same carbon of the steroid ring system and R¹⁶ are as defined for cyclic ketal or cyclic thioketal; and wherein R^{PR} independently are —H or protecting group. In preferred embodiments, (a) R⁵ and R⁶ are —CH₃ in the 6-configuration or R⁵ is —CH₃ in the β-configuration and R⁶ is —H in the 6-configuration and (b) R⁴ together are —OCH₂CH₂O—.

In one preferred embodiment R¹⁰ is present at position C-4 of Formula 2 and is an alkyl group. In another preferred embodiment one R¹⁰ is present at position C-2 and is a suitable monovalent O-linked moiety in the α- or β-configuration or two R¹⁰ are present at position C-2 wherein one R¹⁰ is a suitable monovalent O-linked moiety, and the other R¹⁰ is —H or alkyl. In another preferred embodiment the first hydrogen donor is a hydrogen atom donor provided by hydrogen and Pd(0) or a Pd (II) salt, optionally on a support, more preferably provided by Pd/C, H₂ or Pd(OH)₂/C, H₂. In more preferred embodiments the hydrogen atom donor is provided by hydrogen at between about 1 bar to 3.5 bar or between about 15 psi to 50 psi at between about room temperature (e.g. about 22° C.) to about 40° C.

The androst-3,5-dien-7-one having the structure of Formula 2 may be prepared from a 3β-O-linked steroid of Formula 4, wherein R¹ in the β-configuration is a monovalent O-linked moiety susceptible to elimination by an elimination agent and the other R¹ is in the α-configuration and is —H or an optionally substituted alky and R³, R⁴, R¹⁰ and n in Formula 4 retain their usual meaning from Formula 1. The requisite 3β-O-linked androst-5-en-7-one steroid of Formula 4 may be obtained, after suitable protection, from a corresponding 3β-hydroxy-androst-5-ene-7-one steroid of Formula 1, wherein R¹ in the β-configuration is —OH, the other R¹ is in the α-configuration and is —H or an optionally substituted alkyl; both R² together are ═O and R³, R⁴, R¹⁰ and n retain their usual meaning or by C-7 oxidation to C-7 ═O of an analogous 3β-hydroxy-androst-5-ene steroid wherein both R² are —H. In one embodiment the susceptible monovalent O-liked moiety is an ester, preferably acetate, and the elimination agent is an organic sulfonic acid in non-aqueous solution, preferably an arene-sulfonic acid, more preferably, p-toluene sulfonic acid.

Thus, a reaction sequence, referred to as Method A, to prepare a 3α-hydroxy steroid from a 3β-hydroxyandrost-5-ene steroid that results in overall inversion to the α-configuration of an O-linked moiety at position C-3 of a 3β-O-linked-androst-5-ene steroid derived from the 3β-hydroxyandrost-5-ene steroid, comprises the steps of (1) contacting a suitably protected 3β-O-linked steroid of Formula 4

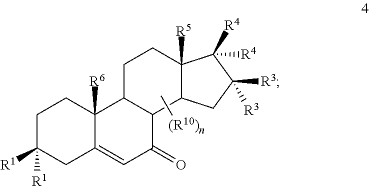

with an eliminating agent, wherein R¹ in the β-configuration is a monovalent O-linked moiety susceptible to elimination from contact with the eliminating agent; the other R¹ in the α-configuration is —H or a suitable optionally substituted alkyl and R³, R⁴, R⁵, R⁶, R¹⁰ and n of the 3β-O-linked steroid is as previously defined for Formula 2, whereby an androst-3,5-diene steroid product is formed; (2) contacting a suitably protected androst-3,5-diene steroid obtained or derived from step 1 with an epoxidizing agent, wherein the androst-3,5-diene steroid has the structure of Formula 2,

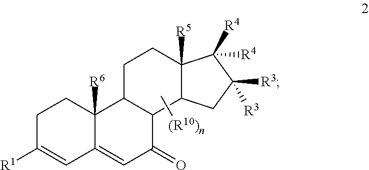

wherein R¹ is —H or optionally substituted alkyl and R³, R⁴, R⁵, R⁶ and R¹⁰ of the androst-3,5-diene is as previously defined for Formula 2 whereby a 3α,4α-epoxy-androst-5-ene is formed; and (3) contacting a suitably protected 3α,4α-epoxy-androst-5-ene obtained or derived from step 2 with a first hydrogen donor wherein the first hydrogen donor is a hydrogen atom donor, wherein the epoxy-androst-5-ene has the structure of Formula 3,

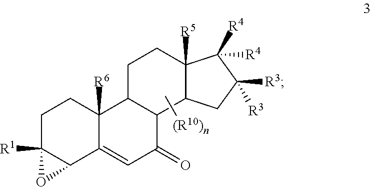

R¹, R³, R⁴, R⁵, R⁶, R¹⁰ and n is as previously defined for Formula 2, whereby a 3α-hydroxy steroid product, optionally after protecting group removal, is formed.

Preferred 3α,4α-epoxy-androst-5-ene steroids prepared from an androst-3,5-diene of Formula 2 as described above include 17,17-ethylenedioxy-3α,4α-epoxy-androst-5-en-7-one, 17,17-di-methoxy-3α,4α-epoxy-androst-5-en-7-one, 17,17-di-ethoxy-3α,4α-epoxy-androst-5-en-7-one, 17,17-propylene-1,3-dioxy-3α,4α-epoxy-androst-5-en-7-one, 3α,4α-epoxy-androst-5-en-7,17-dione, 17,17-tetramethyl-ethylenedioxy-3α,4α-epoxy-androst-5-en-7-one, 17,17-cyclohex-1,2-yl-dioxy-3α,4α-epoxy-androst-5-en-7-one, 17,17-ethylenedioxy-3α,4α-epoxy-androst-5-en-7-one-2β-ol and 17,17-ethylenedioxy-3α,4α-epoxy-androst-5-en-7-one-1α-ol.

In some embodiments a 3α-hydroxy steroid is prepared using a reaction sequence, referred to as Method B, that results in overall inversion of an O-linked moiety at position C-3 in the β-configuration to the α-configuration comprising the step of contacting a 3β-hydroxy steroid having the structure of Formula 1, wherein R¹ in the β-configuration is —OH; R¹ in the α-configuration is —H or a suitable optionally substituted alkyl and R², R³, R⁴, R⁵, R⁶, R⁷, R⁸, R⁹ and R¹⁰ are as previously defined for Formula 2, with an azo-di-carboxylate ester, a tri-substituted phosphine and an organic acid; wherein the molar ratio of the azo-di-carboxylate ester to the 3β-hydroxy steroid is less than 1.5:1 and greater than 1.0:1, whereby a 3α-hydroxy steroid is formed.

In some preferred embodiments the molar ratio of the azo-di-carboxylate ester to the 3β-hydroxy steroid is about 1.3:1. In other preferred embodiments the azo-di-carboxylate ester, tri-substituted phosphine and organic acid are in substantially equimolar amounts. In other preferred embodiments the organic acid is ArC(O)OH, wherein Ar is optionally substituted, which provides an ester at C-3 in the α-configuration that may be hydrolyzed to provide the free 3α-hydroxy substituent. In some preferred embodiments the organic acid is p-nitrobenzoic acid. In some embodiments the an azo-di-carboxylate ester is added to a mixture of the tri-substituted phosphine, organic acid and β-hydroxy steroid at between about 0 to 25° C., preferably between about 0-10° C. In some embodiments the mixture, after adding of the azo-di-carboxylate ester, is warmed to between about 10-25° C.

A 3α-hydroxy androst-5-en-7-one steroid product having the structure of Formula 1 (i.e., R¹ in the α-configuration is —OH and R¹ in the β-configuration is —H or optionally substituted alkyl; R² together as ═O), prepared by Method A or by Method B, after subsequent C-7 oxidation of a suitably protected 3α-hydroxy androst-5-ene obtained therefrom, may be contacted, after suitable protection, with a second hydrogen donor, wherein the second hydrogen donor is hydride donor, to provide a compound having the structure of Formula 1 wherein R¹ in the α-configuration is a monovalent O-lined moiety and R¹ in the β-configuration is —H or optionally substituted alkyl, one R² is a monovalent O-linked moiety and the other R² is —H.

For this step, a suitably protected 3α-hydroxy steroid preferably has its 3α-hydroxyl optionally protected and other hydroxy and ═O functional groups, if present, protected with protecting groups typically employed for hydroxyl and ketone as given in Greene, T. W.

“Protecting groups in organic synthesis” Academic Press, 1981. The optional hydroxy protecting group should be suitable for conditions required to reduce the ═O (ketone) functional group at position 7 and have conditions for this transformation that do not adversely effect other protecting groups already present. Preferred hydride donors as the second hydrogen donor are hydride donors suitable for reducing the ═O functional group at position 7 without removing protecting groups to be retained and is capable of sufficient selectivity to provide 7β-hydroxy or 7α-hydroxy as the predominant isomer if required. Suitable hydroxy protecting groups include ester, usually C₁₋₆ alkyl ester, ether, or silyl ether and the protecting group for other ═O functional groups (e.g., at position C-17) is ketal and the hydride donor as the second hydrogen donor is a borohydride-based reducing agent. Use of a stronger hydride reducing agent would require a hindered ester or substituted methyl ether or silyl ether as the optional hydroxy protecting group to prevent premature loss of the hydroxy protecting group. Preferred ═O (ketone) protecting groups are ketal, such as dimethyl ketal, diethyl ketal or a spiro ketal (i.e., a cyclic ketal) prepared from a glycol or alkanediol such as ethylene glycol, 1,3-propylene glycol or trans-1,2-cyclohexanediol. A preferred suitably protected 3α-hydroxy steroid is a 17,17-ethylenedioxy-androst-5-en-7-one steroid with optional protection of the 3α-hydroxy substituent as an ester, ether or silyl ether optionally having a 16α-ester, 16α-ether, 16α-silyl ether, 16α-fluoro or 16α-alkyl substituent, wherein the esters are preferably a C₂₋₄ ester such as acetate.

Procedures using a hydride donor include reduction with metal hydride based reagents such as the borohydride based reagents that include Zn(BH₄)₂, NaBH₄, optionally with a transition metal salt such as CeCl₃, NiCl₂, CoCl₂ or CuCl₂, L-Selectride (lithium tri-sec-butylborohydride) or N-Selectride (sodium tri-sec-butylborohydride). Lithium aluminum hydride based or sodium aluminum hydride reagents may also be used although selectivity may suffer due to the reducing strength of such reagents. This may be ameliorated by using lithium aluminum hydride based reagents having alkoxy ligands to aluminum to reduce reactivity. Such reagents have the general formula LiAl—Hₙ(OR)₄₋ₙ, where n=1, 2, 3, R is C₁₋₆ alkyl and include LTMA (lithium triethoxyaluminum hydride LTEAH (lithium triethoxyaluminum hydride), RED-AL (Sodium bis(2-methoxyethoxy)aluminium hydride). Reduction using borohydride based reagents may be conducted in alcohol solvents whereas reductions with aluminium hydride based reagents require an ether solvent such as THF. Selectivity may be improved, particularly for the aluminum hydride reagents, by conducting the reaction at temperature of between 0° C. to −78 C with lower temperatures being more suitable for the aluminum hydride reagents.

Additionally, 3α-hydroxy steroids, including 3α-hydroxy-androst-5-ene, 3α-hydroxy-androst-5-en-7-one, 3α-hydroxy-5α-androstane and 3α-hydroxy-5α-androstan-7-one steroids having di-substitution at C-17, wherein one substituent in the β-configuration is a monovalent O-linked moiety and the other substituent in the α-configuration is optionally substituted alkyl, optionally substituted alkenyl or optionally substituted alkynyl, may be effected by contacting a suitably protected 3α-hydroxy steroid prepared or derived from a steroid product of Method A or Method B and having a ═O moiety at position C-17 with an suitable organometallic agent whereby the organometallic agent adds to the ketone at position C-17. These steroids, which are prepared or derived from a steroid product of Method A or Method B, to be suitably protected include androst-5-en-17-one-3α,7α-diol, androst-5-en-17-one-3α,713-diol, 3α-DHEA and their 5α-androstane analogs obtained by saturation of the Δ⁵ functional group in these androst-5-ene steroids with a third hydrogen donor.

Procedures to prepare an 3α-hydroxy steroids having disubstitution at C-17 wherein one C-17 substituent in the β-configuration is —OH and the other C-17 substituent in the α-configuration is —C≡CH include for example contacting a suitably protected 3α-hydroxy steroid precursor obtained or derived from Method A or Method B having a ═O moiety at position C-17 with sodium acetylide, lithium acetylide (as its ethylene diamine complex), ethynyl magnesium halide (e.g., chloride or bromide) or ethynyl zinc halide, as for example in U.S. Pat. No. 2,243,88 (specifically incorporated by reference herein), in diethylether or other ether solvents such as tetrahydrofuran, 1,2-dimethoxyethane, 2-methoxyethylether and the like.

In one embodiment a 3α-hydroxy steroid having disubstitution at C-17 is prepared by contacting a suitably protected 3α-hydroxy steroid having a ═O moiety at position C-17, such as androst-5-en-17-one-3α,7α-diol, androst-5-en-17-one-3α,713-diol or 3α-DHEA that is suitably protected, with an in situ preparation of an acetylene anion. The acetylide may be prepared in situ by contacting acetylene with an amide anion (e.g., NaNH₂) in a hydrocarbon solvent such as benzene, toluene or xylene, as for example in U.S. Pat. No. 2,251,939 (specifically incorporated by reference herein), with sodium or potassium metal in liquid ammonia, as for example in U.S. Pat. No. 2,267,257 (specifically incorporated by reference herein), or by contacting a mono-silyl protected acetylene such as trimethylsilyl acetylene with an organolithium reagent. Suitable organolithium reagents include commercially available n-butyl lithium, sec-butyl lithium, methyl lithium, t-butyl lithium or phenyl lithium or can be prepared by reaction of an alkyl or aryl bromide with metallic lithium in an inert solvent such as diethyl ether or tetrahydrofuran.

Suitable protection for 3α-hydroxy steroids such as androst-5-en-17-one-3α,7α-diol, androst-5-en-17-one-3α,713-diol or 3α-DHEA for reactions using organometallic agents will have hydroxyl protecting groups that are typically used in carbanion chemistry and can be introduced under conditions compatible with an allylic alcohol and may be removed under conditions that are compatible with the presence of a terminal alkyne and an allylic alcohol. Such protecting groups will usually be removable under neutral or mildly acidic conditions, typically between about pH 3-7. Preferred protecting groups are silyl ethers of the formula (R¹³)₃SiO— (i.e., —OH transformed to —OR^{PR} wherein R^{PR} is —Si(R¹³)₃) wherein R¹³ independently are aryl or C₁₋₆ alkyl and include trimethylsilyl, triethylsilyl, t-butyldimethylsilyl, isopropyldimethylsilyl, t-butyldiphenylsilyl, methyldiisopropylsilyl, methyl-t-butylsilyl, tribenzylsilyl and triphenylsilyl ether. Some substituted methyl ethers may be used and include 2-(trimethylsilyl)-ethoxymethyl ether (SEM ether), tetrahydropyranyl ether (THP ether), tetrahydrothiopyranyl ether, 4-methoxy-tetrahydropyranyl ether, 4-methoxytetrahydrothiopyranyl ether, tetrahydrofuranyl ether and tetrahydrothiofuranyl ether. Some optionally substituted ethers may be used as hydroxy protecting groups and include 1-ethoxyethyl ether and t-butyl ether. Preferred hydroxy protecting groups have lower steric demands, such as trimethylsilyl ether and allow for simultaneous protection of the 3α- and 7α/β-hydroxy groups (if present).

Procedures to prepare 3α-hydroxy steroids and other 3α-O-linked steroid having O-linked substitution at position C-16 may introduce a monovalent O-linked substituent at this position prior to or after the inversion of configuration at position C-3 of a 3β-hydroxy steroid precursor that provides a corresponding 3α-hydroxy steroid by Method A or Method B. In one method of introducing a monovalent O-linked substituent to position C-16, an appropriately protected 3β- or 3α-hydroxy steroid having a ═O moiety at position C-17 is brominated to provide a C-16 bromo intermediate, which is then subjected to controlled hydrolysis. In another method the ketone at position C-17 is enolized to provide a silyl enol ether, which is then oxidized to provide a C16-C17 epoxide whereupon on hydrolysis provides the corresponding 3β- or 3α-hydroxysteroid having a ═O (ketone) moiety at position 17 and a monovalent O-linked moiety at position C-16 in the α-configuration.

Methods to prepare 3α-O-linked and 3b-O-linked steroids with C16-bromo substitution are provided in Scheme 1, wherein R² and R¹⁰ independently are —H, a suitable O-linked moiety or a suitable monovalent C-linked moiety, R³ is —H or a suitable monovalent C-linked moiety, and —H at position C-5 (if present) is in the α-configuration, optionally wherein the monovalent C-linked moieties independently are suitable optionally substituted alkyl moieties. Introduction of bromine at position C-16 of an androst-5-ene or 5α-androstane steroid to provide compounds having structure A is accomplished in one method by direct alpha bromination of a C17-ketone using Br₂ or CuBr₂. Bromination at C-16 to provide steroids of structure A is also accomplished by another method indirectly through formation of an enol ester, such as an enol acetate, represented by Intermediate B, wherein —OR═—OAc or through a silyl enol ether, wherein —OR is —OSi(OR¹³)₃. Exemplary conditions for bromination of steroids having a ═O moiety at position C-17 are adaptable from those found in the following cited documents.

Scheme 1. Introduction of a halogen or monovalent O-linked substituent into a 3α-O-linked androst-5-ene or 5α-androstane steroid at position C-16.

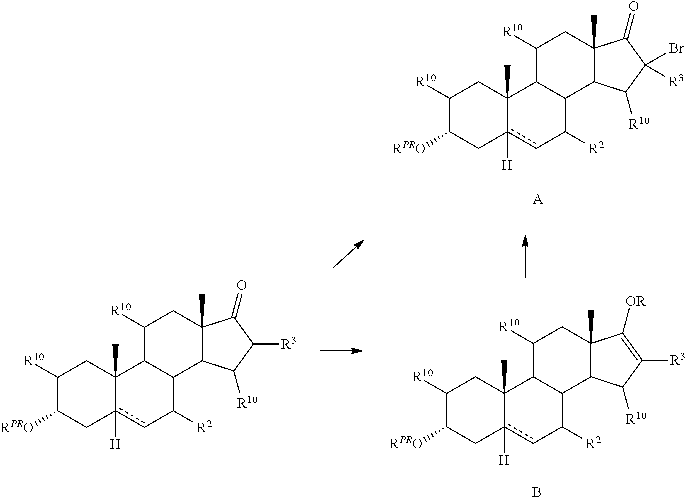

Specific methods for preparing 3α-O-linked androst-5-en-17-one steroids with 16-bromo substitution (Intermediate A) are adapted from procedures found in the following documents. Direct Bromination of C17-one compounds: Numazawa, M., et al. J. Org. Chem. 47(21): 4024-9 (1982); Dubey, S., et al. Med. Chem. Res. 14(4): 229-240 (2005); Grosek, G., et al. Bull. Pol. Acad. Sci. 34: 7-8 (1966); Piplani, P. et al Ind. J. Chem. Sect. B 39(5): 363-7 (2000); Numazawa, M., et al. Chem. Pharm. Bull. (Jpn) 33(2): 865-8 (1985); Numazawa, M., et al. Ibid.: 48(9): 1359-62 (2000); Abou-Gharbia, M., et al J. Pharm. Sci. 70(10); 1154-7 (1981); Shi, B. et al. Angew. Chem. Intl. Ed. 43(33): 4324-27 (2004) (using CuBr₂); Fajikos Coll. Czech. Chem. Comm. 20: 312-331 (1955); Cantineau, R., et al. Steroids 37(2): 177-194 (1981) (using Br₂). Bromination of C17 Enol Ester (Intermediate B, —OR═—OC(O)R′): X═Br: Faijikos, Coll. Czech. Chem. Comm. 23: 1559-1567 (1958); Ibid. 24: 766-777 (1959) (using NBS, CCl₄); Pappo, et al. J. Am. Chem. Soc. 78: 6347-6351 (1956); Anderson, A., et al. J. Med. Chem. 43(22): 4118-4125 (2000); Nambara, T. Chem. Pharm. Bull. (Jpn) 12(10): 1253-58 (1964); Petersen, L. P., et al. Steroids 13: 793-802 (1969); Ellis, et al. J. Chem. Soc. 1958: 800-2 (1958); Marwah, P., et al. Bioorg. Med. Chem. 14(17): 5933-5947 (2006) (using Br₂). Bromination of a Silyl Enol Ether (Intermediate B, —OR═—OSi(R′)₃): Liu, A., et al. J. Med. Chem. 35(11): 2113-2129 (1992) (using NBS).

The 16-bromo substituent in structure A may then be hydrolyzed to provide —OH as the O-linked substituent at position C-16 using, for example, NaOH in DMF or py as described in Numazawa, et al. J. Org. Chem. 47(21): 4024-9 (1982); Numazawa, et al. Steroids 45(5): 403-410 (1985); Numazawa, et al. J. Am. Chem. Soc. 102(16): 5402-4 (1980). The 16-bromo substituent in structure A may also be displaced with various nucleophiles to introduce other monovalent O-linked moieties substituents at position C-16 such as ethoxy or methoxy or another halogen such as fluoro.

In some embodiments 3α-O-linked steroids are prepared from 3β-hydroxy androst-5-en-7-one steroids using Method A according to the reaction sequence of Scheme 2. In this reaction sequence, the 3β-hydroxy substituent in a 3β-hydroxy androst-5-en-7-one steroid, represented by structure C, is converted to another monovalent O-linked substituent in the β-configuration, preferably an ester that is capable of elimination to form an androst-3,5-dien-7-one steroid having structure D.

Elimination reaction conditions (i.e., elimination agents as defined herein), suitable for elimination of a susceptible 3β-O-linked substituent in an androst-5-en-7-one steroid that are also suitable for retaining other substituents and functional groups in the steroid, or for desired concurrent deprotection or protection event (s), to provide an androst-3,5-dien-7-one steroid represented by structure D include a Brønsted acid in an alcoholic solvent, such as HCl in ethanol, H₂SO₄ in methanol, perchloric acid in methanol, or an alkyl or aryl sulfonic acid in a suitable solvent, such as p-toluenesulfonic acid in ethylene glycol or dioxane, as used, for example, in procedures adaptable from Reichstein, Helv. Chim. Acta 22: 1160-3 (1939), Marshall, J. Am. Chem. Soc. 79: 6303-7 (1957), Butenandt, et al. Chem. Ber. 71: 1316-1321 (1936); U.S. Pat. No. 2,824,882 (specifically incorporated by reference herein), Romo, J. Org. Chem. 17: 1413-1417 (1952), Fischer, J. Liebig's Ann. 636: 88-104 (1960), Okamura, et al., J. Org. Chem. 43(4): 574-580 (1978), Marwah, et al. Bioorg. Chem. 30(4): 233-248 (2002). Other suitable elimination agents include a hydroxide base in an alcoholic solvent, such as KOH in methanol or ethanol, a hindered base in a paretic solvent or a Lewis acid, as used, for example, in procedures adaptable from U.S. Pat. No. 2,170,124 (specifically incorporated by reference herein), Tanabe, et al. Chem. Pharm. Bull. (Jpn) 7: 811-5 (1959), Marker, et al. J. Am. Chem. Soc. 69: 2167-2189 (1947), Solyom, Acta Chim. Hung. 125(1): 23-8 (1988), Lederer, Bull. Chim. Soc (Fr) 1965: 1298-1308 (1965).

Intermediate D is then epoxidized with an epoxidizing agent, preferably with a peracid such as m-chloroperbenzoic acid (mcpba) to form a 3α,4α-epoxy-androst-5-ene-7-one steroid having structure E. Other ketones (i.e., ketones not at position C-7) that may be present in C or intermediate B, such as a ketone at position C-17, are typically protected, as for example as a ketal, prior to epoxidation to avoid Bayer-Villiger oxidation. Contacting intermediate E with a hydrogen donor capable of selective reduction of the 3α,4α-epoxy functional group relative to the Δ⁵ and C7-one functional groups by reductively opening this epoxide at position C-4 with retention of configuration at position C-3 provides, after deprotection, a 3α-hydroxy-androst-5-en-7-one steroid of structure F (wherein R¹ is —OH), thereby completing the inversion of configuration at position C-3 of a 3β-hydroxy-androst-5-ene steroid precursor. Contacting intermediate E with a hydrogen donor capable of selective reduction of the 3α,4α-epoxy functional group relative to the Δ⁵ and concomitant reduction of the C7-one functional group provides, after deprotection, a 3α-hydroxy-androst-5-en-7-ol steroid of structure G (wherein R¹ is —OH and one R² is —OH and the other R² is —H),

Scheme 2. Preparation of 3α-O-linked steroids by Method A from a 3β-hydroxy-androst-5-ene precursor

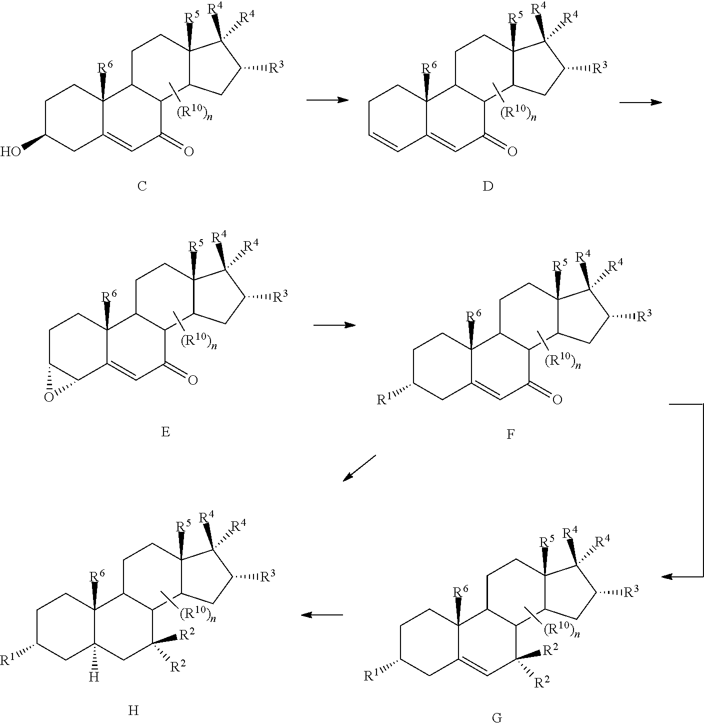

The reductive epoxide opening in E may be affected by a suitable reducing agent (a first hydrogen donor). A suitable first hydrogen donor is capable of reductive epoxide opening at position C-4 of a 3α,4α-epoxy-androst-5-ene-7-one steroid with retention of configuration at position C-3 under reaction conditions that substantially do not effect unintended chemical transformations of other substituents or functional groups in the steroid such as premature protecting group removal with or without concomitant C-7 one reduction. Suitable first hydrogen donors for reductive epoxide opening to provide 3α-hydroxy-androst-5-en-7-one steroids, represented by structure F, include a hydrogen atom donor, wherein the hydrogen atom donor is, for example, hydrogen gas or formic acid in the presence of a Pd or Pt catalyst, such as Pd(0) optionally absorbed onto a solid support, such as carbon black, optionally in the presence of a hindered base or a carbonate salt, such as potassium or strontium carbonate. Other hydrogen atom donors include Pd(dba)₂, formic acid and a hindered base (Tsuji-Trost reaction), lithium in liquid ammonia or Cr(OAc)₂ or Zn in acetic acid. Reaction conditions for these other hydrogen atom donors are adaptable from the procedures in Robinson, et al. J. Org. Chem. 37(4): 565-568 (1972); Irmsher, et al. Chem. Ber. 97; 3363-3373 (1964); Roussi, et al. Eur. J. Org. Chem. 18: 3952-3961 (2005); Knowles, J. Am. Chem. Soc. 79: 3212-4 (1957). Other suitable reducing agents for reductive epoxide opening also include hydride donors such as lithium aluminum hydride (LAH) in a polar aprotic solvent such as tetrahydrofuran (THF), dioxane or diethyl ether, which effect reductive 3α,4α-epoxy opening concomitant with 7-one reduction to form 3α,7ζ-di-hydroxy-androst-5-ene steroid represented by structure G wherein one R² is —OH and the other R² is —H. Reaction conditions using these hydride donors are adaptable from the procedures in Stary, et al. Coll. Czech. Chem. Comm. 50(5): 1227-1238 (1985); Kim, et al. Tet. 53(24): 8129-8136 (1997). A preferred hydrogen atom donor is hydrogen gas in the presence of Pd(0)/C and K₂CO₃. A preferred hydride donor is LAH in THF.

Other 3α-O-linked-androst-5-ene steroids may be prepared from a suitably protected 3α-hydroxy-androst-5-en-7-one steroid, 3α,713-di-hydroxy-androst-5-ene or 3α,7α-di-hydroxy-androst-5-ene steroid having structure F or G by contacting a 3α-hydroxy-androst-5-en-7-one steroid product from Method A after suitable protection with a suitable electrophile or a suitable hydrogen donor that effects reduction of the 7-one functional group to C7-hydroxy (a second hydrogen donor). This second hydrogen donor will provide a 3α,713-di-hydroxy-androst-5-ene steroid product, a 3α,7α-di-hydroxy-androst-5-ene steroid product or a mixture thereof, represented by structure G, which may be separated by standard chromatographic methods. A suitably protected 3α-hydroxy-androst-5-en-7-one steroid may also be contacted with a organometallic agent having the structure R²-M, wherein M is a suitable optionally substituted alkyl, alkenyl or alkynyl moiety and M is a Group 1, Group 2, or a transition metal to provide a product of structure G wherein one R² is —OH and the other R² is derived from the organometallic agent.

3α-O-linked-5α-androstane steroids represented by structure H may be prepared from suitably protected 3α-O-linked-androst-5-ene steroids through contact of steroids having structure F or G with a reducing agent capable of saturating the Δ⁵-functional group (a third hydrogen donor) that may or may not reduce other functional groups present in the molecule depending on reaction conditions and protecting group strategy. For example, an androst-5-en-7-one steroid may be reduced from contact with a third reducing agent to provide a 3α-O-linked-74-hydroxy-5α-androstane or a 3α-O-linked-5α-androstan-7-one by complete saturation of the α,β-unsaturated functional group or selective Δ⁵ saturation.

Steroids with C17-disubstitution with structures F, G or H wherein one R⁴ is -a monovalent O-linked moiety and the other R⁴ is a monovalent C-linked moiety, such as an optionally substituted alkyl, optionally substituted alkenyl or optionally substituted alkynyl, may be prepared either using a 3β-hydroxy-androst-5-ene precursor already containing C17-disubstitution or by contacting a suitably protected androst-5-en-17-one of structure G or H wherein both R⁴ together are ═O with a organometallic agent having the structure R⁴-M, wherein M is a suitable optionally substituted alkyl, alkenyl or alkynyl moiety and M is a Group 1, Group 2, or a transition metal and optionally quenching the reaction with an electrophile. The C17-disubstituted steroid thus formed has one R⁴ as —OH or another monovalent O-linked moiety provided by the quenching electrophile and the other R⁴ derived from the organometallic agent.

Suitable 3β-hydroxy-androst-5-en-7-one steroid precursors for Scheme 2 may be obtained by C7-oxidation of a suitably protected 3α-O-linked-androst-5-ene-7ζ-ol or 3α-O-linked-androst-5-ene unsubstituted at position C-7, wherein the 3α-O-linked substituent is —OR^{PR} wherein R^{PR} is a protecting group. Procedures for this oxidative transformation include microbial oxidation as described in Wuts, P.G.M. “A chemobiological synthesis of eplerenone” Synlett (3): 418-422 (2008); oxidation with oxo-chromium based reagents [e.g., see Koutsourea, et al., “Synthetic approaches to the synthesis of a cytostatic steroidal B-D bilactam” Steroids 68: 569-666 (2003) and Condom, et al., “Preparation of steroid-antigens through positions of the steroid not bearing functional groups” Steroids 23: 483-498 (1974)], peroxide assisted allylic oxidation [e.g., see Marwah, P., et al. “An economical and green approach for the oxidation of olefins to enones” Green Chem. 6: 570-577 (2004) and Marwah, P., et al., “Ergosteroids IV: synthesis and biological activity of steroid glucuronosides, ethers and alkylcarbonates” Steroids 66: 581-595 (2001)] and oxidation with N-hydroxysuccimimide/AIBN [e.g., see Lardy, et al. “Ergosteroids II: Biologically active metabolites and synthesis derivatives of dehydroepiandrosterone” Steroids 63:158-165 (1998)].

In the structures of Scheme 2, R³ is —H, a suitable halogen, optionally fluoro, a suitable monovalent C-linked moiety, optionally C₁₋₆ alkyl, or a suitable monovalent O-linked moiety, one R⁴ is a suitable monovalent O-linked moiety and the other R⁴ is —H, a suitable monovalent O-linked moiety or a suitable monovalent C-linked moiety, optionally wherein the monovalent C-linked moiety is a suitable optionally substituted alkyl, optionally substituted alkenyl or optionally substituted alkynyl, optionally C₁₋₆ alkyl, C₂₋₆ alkenyl or C₂₋₆ alkynyl, or both R⁴ together define a cyclic ketal, optionally a divalent O-linked moiety having the structure of —O—[C(R₁₆)], —O—, wherein n=2 or 3 and R¹⁶ independently are —H or C₁₋₄ alkyl; R⁵ and R⁶ are —H or optionally substituted alkyl independently selected, optionally —CH₃ or CH₂OR^{PR}; wherein the monovalent O-linked moieties independently are —OH, an ester, optionally a C₁₋₆ ester, an ether, optionally a C₁₋₆ ether, silyl ether, optionally —OSi(R¹³)₃, or —OR^{PR}, wherein R¹³ independently are alkyl or aryl, optionally C₁₋₄ alkyl or phenyl and R^{PR} independently are —H or a protecting group.

Preferred androst-3,5-dien-7-ones for epoxidation with a peracid such as m-chloroperbenzoic acid have substituents in structure D, and are thus preferred substituents in 3β-hydroxy-androst-5-ene precursors of structure C, that favor or do not disfavor approach of the epoxidizing agent to the α-face of this steroid in comparison to the β-face and have R⁴ together as a divalent oxygen substituent having the structure of —O[C(R¹⁶)₂]ₙO—, wherein n=2, 3; R¹⁶ are as defined for cyclic ketal, with n=2 and R¹⁶ are —H (i.e., —OCH₂CH₂O—) preferred. When R⁶ in D is alkyl or an optionally substituted alkyl having the structure of —CH₂—R⁶′ wherein R⁶′ is a monovalent C-linked moiety or a monovalent O-linked moiety that is not —OH or a carbamate or is an ester, an ether, a silyl ether or a carbonate, steric hindrance from R⁶ is expected to favor epoxidation to the α-face and to dominate over any peracid directing group effects to the β-face of the androst-3,5-dien-7-one steroid to provide predominately the desired 3α,4α-epoxy-androst-5-en-7-one steroid product. When R⁶ is —H or R⁶′ is —OH, R¹⁰ substituent(s) at positions C-1 or C-2, as described in the following, may be required to compensate for the directing effect of these R⁶′ moieties or the absence of steric hindrance from R⁶ so that α-face epoxidation remains predominant over β-face epoxidation. This regioselectivity for α-face epoxidation is expected to be enhanced with a R¹⁰ substituent that is —OH pseudo-equatorial at position C-2 or pseudo-axial at position C-1 of D (i.e., in a C precursor R¹⁰ at C-2α or C-1α is —OH) due to the directing group effect of this substituent on peracid epoxidation. When R¹⁰ is at these positions and is an ether, a silyl ether or an ester, steric effects predominate, and are thus expected to weaken the predominance for α-face epoxidation. Selectivity for α-face epoxidation is also expected to be weakened when there is a R¹⁰=—OH substituent pseudo-axial at position C-2 of D (i.e., in a C precursor R¹⁰ at C-2β is —OH) due to the directing group effect of this substituent for β-face peracid epoxidation. When this R¹⁰ is an ether, a silyl ether or an ester, the steric effects of these substituents are expected to predominate over any directing effects thus reinforcing the steric effect of R⁶′ to enhance α-face epoxidation. When there is an R¹⁰ substituent that is a monovalent C-linked moiety at position C-2 or pseudoaxial at position C-1 of D (i.e., in a C precursor the R¹⁰ substituent is at C-2α/β or C-1α) the steric hindrance from these substituents opposes that of R⁶′ and is thus expected to weaken predominance for α-face epoxidation.

In consideration of the foregoing preferred substituents in structure C for use in Method A (i.e., preferred precursors to obtain D) due to their effect on epoxidation on structure D are (1) when R⁶ is optionally substituted alkyl having the structure of —CH₂—R⁶′, wherein R⁶′ is —H (i.e., R⁶ is —CH₃), a suitable monovalent C-linked moiety, a suitable halogen or a suitable ester, ether or silyl ether, preferably R⁶′ is C₁₋₆ ester, —H or —CH₃ (i.e., R⁶ preferably is, —CH(C₁₋₆ ester), —CH₃ or —CH₂CH₃) (1) one R¹⁰ is present at position C-2 in the β-configuration and is a suitable monovalent C-linked moiety or a suitable ester, ether or silyl ether, preferably this R¹⁰ is C₁₋₆ alkyl or C₁₋₆ ester, or is absent or R¹⁰ is present in the α-configuration and is —OH or is absent, and if R¹⁰ at position C-1 is present and is in the α-configuration this substituent is —OH, —CH₃ or —OAc or if present in the β-configuration this substituent is a suitable monovalent C-linked moiety, preferably C₁₋₆ alkyl, a suitable halogen, preferably fluoro or a suitable O-linked moiety, preferably —OH or C₁₋₆ ester and (2) when R⁶ is optionally substituted alkyl having the structure of —CH₂—R⁶′, wherein R⁶′ is —OH (a) an R¹⁰ substituent is present at position C-2 in the α-configuration and is —OH or an R¹⁰ is present at position C-2 in the β-configuration and is a suitable monovalent C-linked moiety, preferably C₁₋₆ alkyl or a suitable ester or ether and, if a R¹⁰ substituent is present C-1, this R¹⁰ substituent is —OH in the α-configuration or if an R¹⁰ substituent is present in the β-configuration at position C-1 this substituent is a suitable O-linked moiety, preferably —OH or C₁₋₆ ester, or a suitable C-linked moiety, preferably C₁₋₆ alkyl or (b) one R¹⁰ substituent is present in the α-configuration at position C-2 and is —CH₃ or —OAc and another R¹⁰ is present in the β-configuration at position C-2 and is a suitable monovalent C-linked moiety, or a suitable ester, ether or silyl ether, preferably this R¹⁰ substituent is C₁₋₆ alkyl or C₁₋₆ ester, and no R¹⁰ substituents are present at position C-1 or if present this substituent is in the α-configuration and is —OH or is in the β-configuration and is a suitable C-linked moiety, preferably C₁₋₆ alkyl or a suitable O-linked moiety, preferably —OH or C₁₋₆ ester or (c) no R¹⁰ substituent is present at position C-2 and one R¹⁰ is present at position C-1 in the α-configuration and is —OH and another R¹⁰ at position C-1 in the β-configuration if present is a suitable O-linked moiety, preferably —OH or C₁₋₆ ester, or a suitable C-linked moiety, preferably C₁₋₆ alkyl and (3) when R⁶ is —H (a) one R¹⁰ is present at position C-2 in the β-configuration and is a suitable monovalent C-linked moiety, or a suitable ester, ether or silyl ether, preferably this R¹⁰ substituent is C₁₋₆ alkyl or C₁₋₆ ester and another R¹⁰ substituent in the b-configuration is not present and if R¹⁰ is present at position C-1 this substituent is in the α-configuration and is —OH or is in the β-configuration and is a suitable monovalent C-linked moiety, preferably C₁₋₆ alkyl or a suitable O-linked moiety, preferably —OH, or an ester, preferably C₁₋₆ ester or (b) one R¹⁰ is present in the α-configuration at position C-2 and is —OH and R¹⁰ if present at position C-1 is in the β-configuration and is a suitable monovalent C-linked moiety, a suitable halogen or a suitable monovalent O-linked moiety, preferably this R¹⁰ substituent is C₁₋₆ alkyl, fluoro, —OH or C₁₋₆ ester or (c) no R¹⁰ substituent is present at position C-2 and R¹⁰ is present at position C-1 in the α-configuration and is —OH and if present another R¹⁰ substituent at position C-1 is in the β-configuration and is a suitable C-linked moiety, preferably C₁₋₆ alkyl and in (1), (2) or (3) both R⁴ together are —OCH₂CH₂O— and R⁵ is —H or a suitable optionally substituted alkyl, preferably —CH₃, —CH₂CH₃ or CH₂OH.

In one embodiment androst-3,5-dien-7-one steroids of structure 6, 17,17-ethylenedioxy-3α,4α-epoxy-androst-5-en-7-one steroids of structure 7, 17,17-ethylenedioxy-3α-hydroxy-androst-5-en-7-one steroids of structure 8 and 3α-hydroxy-androst-5-en-7,17-dione steroids of structure 9 of Scheme 3 are intermediates useful in the preparation of 3α-hydroxy steroids, and other 3α-O-linked steroids derivable therefrom. These intermediates and are prepared using the reaction sequence of Method A by way of a 3β-acyloxy-androst-5-ene-7,17-dione steroid, such as a 3β-acetoxy-androst-5-ene-7,17-dione steroid of structure 5, according to Scheme 3, wherein R³ is —H or a suitable halogen, optionally fluoro, a suitable monovalent C-linked moiety, optionally a suitable optionally substituted alkyl, or a suitable monovalent O-linked moiety, optionally —OH or a suitable ester, ether or silyl ether; R⁹ is —CH₂—, —CH(α-OH)—, —CH(β-ester), —CH(β-silyl ether) or CH(β-alkyl); and R¹⁰ is at position C-1 in the α-configuration and is —H or —OH or in the β-configuration is —H, a suitable monovalent C-linked moiety, optionally a suitable optionally substituted alkyl, or a suitable monovalent O-linked moiety, optionally —OH, ester, ether or silyl ether, wherein optionally substituted alkyl, ester, ether or silyl ether independently are optionally C₁₋₆ alkyl, C₁₋₆ ester, C₁₋₆ ether or —OSi(R¹³)₃, wherein R¹³ independently are C₁₋₄ alkyl or phenyl.

Scheme 3. Preparation of 3α-hydroxy-17,17-ethylenedioxy-androst-5-en-7-one and 3α-hydroxy-androst-5-en-7,17-dione steroids by Method A from a 3β-acyloxy-androst-5-en-7,17-dione precursor

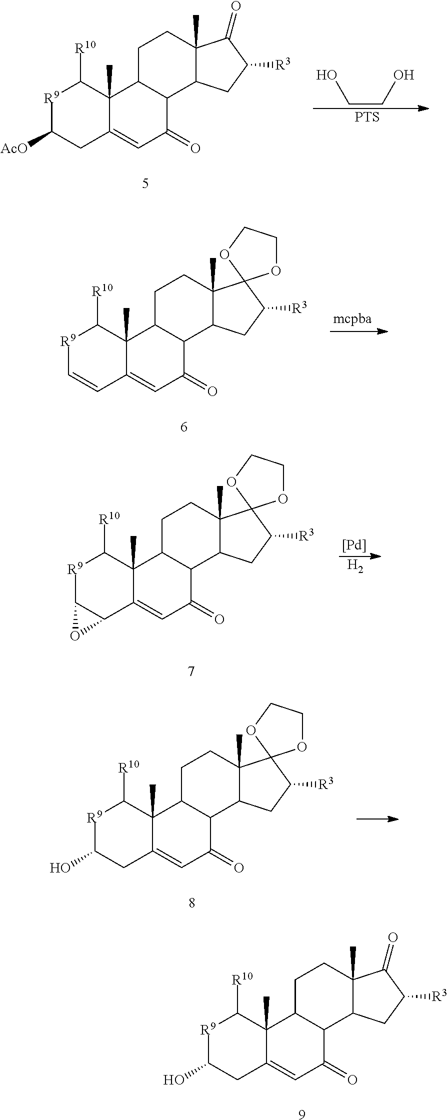

PTS=p-toluene-sulfonic acid; mcpba=m-chloro-perbenzoic acid

In some embodiments of Method A, 17,17-ethylenedioxy-3α-hydroxy-androst-5-en-7-one steroids (8) and 3α-hydroxy-androst-5-en-7,17-dione steroids (9) are prepared according to Scheme 3, wherein R³ is —H, a suitable halogen, a suitable monovalent O-linked moiety or a suitable optionally substituted alkyl, R⁹ is —C(R¹⁰)₂— wherein R¹⁰ independently are —H, a suitable monovalent O-linked moiety or optionally substituted alkyl and R¹⁰ at position C-1 is —H, a suitable monovalent O-linked moiety, a suitable optionally substituted alkyl or a suitable halogen, wherein the suitable monovalent O-linked moieties independently are —OH or a suitable ester, ether or silyl ether and the suitable halogens are optionally chloro, bromo or fluoro. In a particular example 17, 17-ethylenedioxy-3α-hydroxy-androst-5-en-7-one (8a) and 3α-hydroxy-androst-5-en-7,17-dione (9a) were prepared according to Scheme 3 wherein R⁹ is —CH₂— and R₃, R₁₀ at position C-1 are —H.

Scheme 3-1. Preparation of 3α-hydroxy-androst-5-ene steroids having a monovalent O-linked moiety at position C-7 and-or having di-substitution at C-17 from steroid precursors obtained from Method A

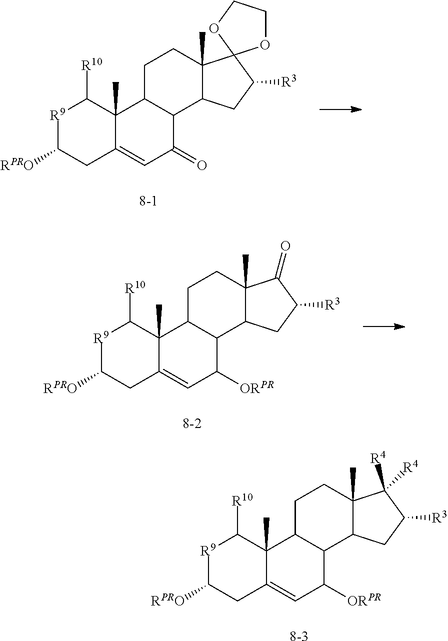

Additional 3α-hydroxy-androst-5-ene steroids having a monovalent O-linked moiety at position C-7 and steroids additionally having di-substitution at C-17 are prepared from steroid products of Scheme 3 according to the reaction sequence of Scheme 3-1 wherein —R^{PR} independently are —H or a protecting group; R⁴ in the β-configuration is a monovalent O-linked moiety and R⁴ in the α-configuration is optionally substituted alkyl, optionally substituted alkenyl or optionally substituted alkynyl. Introduction of the monovalent O-linked substituent at position C-7 may be effected by contacting a suitably protected 3α-hydroxy-androst-5-en-7,17-dione (e.g., 8-1) with a second hydrogen donor such as a hydride donor followed optionally by contacting the product of this reduction with an electrophile whereby a 3α,7ζ-di-O-linked androst-5-ene steroid is obtained.

By contacting a 3α-O-linked-androst-5-en-7-one steroid such as 8a with a second hydrogen donor, wherein the second hydrogen donor is a hydride donor, a hydroxy group in the α or β configuration at position C-7, dependent on the identity of the hydride donor and reaction conditions employed as described elsewhere herein, is obtained. Subsequent deprotection then gives predominately either androst-5-en-17-one-3α,7α-diol or androst-5-en-17-one-3α,7β-diol or a mixture thereof, which can be separated by standard chromatographic methods to provide the individual epimers.

Introduction of disubstitution at position C-17 may be effected by contacting a suitably protected 3α-hydroxy steroid having a ═O moiety at position C-17 (e.g., 8-2) wherein R^{PR} are suitable protecting groups, where the suitably protected 3α-hydroxy steroid is, for example, a suitably protected androst-5-en-7,17-dione-3α-ol, androst-5-en-17-one-3α,7α-diol, androst-5-en-17-one-3α,713-diol or 3α-DHEA, with an suitable organometallic agent.

Procedures to prepare 3α-hydroxy-androst-5-ene steroids having disubstitution at C-17, wherein one C-17 substituent in the β-configuration is —OH and the other C-17 substituent in the α-configuration is —C≡CH from a 3α-hydroxy steroid prepared according to the reaction sequence of Scheme 3-1 include for example contacting a suitably protected 3α-hydroxy steroid precursor having a ═O moiety at position C-17 with sodium acetylide, lithium acetylide (as its ethylene diamine complex), ethynyl magnesium halide (e.g., chloride or bromide) or ethynyl zinc halide, as for example in U.S. Pat. No. 2,243,88 (specifically incorporated by reference herein), in diethylether or other ether solvents such as tetrahydrofuran, 1,2-dimethoxyethane, 2-methoxyethylether and the like.

5α-Androstane steroids may be obtained from the androst-5-ene steroids prepared from the reaction sequences of Scheme 3 or Scheme 3-1 by contacting these steroids having suitable protection with a third hydrogen donor such as a hydrogen atom donor wherein the Δ⁵ functional group is reduced whereby a 5α-androstane steroid is produced with or without concomitant C-7 ketone reduction.

In other embodiments 16α-O-linked and 16α-C-linked analogs of 6, 7, 8 or 9 are intermediates obtainable by Method A that are useful in preparation of biologically active 3α-hydroxy steroids, and other 3α-O-linked steroids derivable therefrom. Examples of such intermediates are 17,17-ethylenedioxy-androst-5-en-7-one-3α,16α-diol, 17,17-ethylenedioxy-16α-acetoxy-androst-5-en-7-one-3α-diol, 17,17-ethylenedioxy-16α-fluoro-androst-5-en-7-one-3α-ol, 17,17-ethylenedioxy-16α-methoxy-androst-5-en-7-one-3α-ol, 17,17-ethylenedioxy-16α-methyl-androst-5-en-7-one-3α-ol, 17,17-ethylenedioxy-16α-propyl-androst-5-en-7-one-3α-ol and 17,17-ethylenedioxy-16α-(prop-2-yl)-androst-5-en-7-one-3α-ol.

In some embodiments, 3α-O-linked steroids are prepared from 3β-hydroxy androst-5-ene steroids using Method B according to the reaction sequence of Scheme 4, wherein R³ is —H, fluoro, bromo, chloro, a suitable monovalent O-linked moiety or a suitable mono valent C-linked moiety; one R⁴ in the β-configuration is a suitable monovalent O-kinked moiety, the other R⁴ in the α-configuration is —H, a suitable monovalent O-linked moiety or a suitable monovalent C-linked moiety or both R⁴ together are a divalent O-linked moiety, preferably ═O or —OCH₂CH₂O—; R⁵ and R⁶ independently are —H or a suitable optionally substituted alkyl, preferably R⁵ and R⁶ are —CH₃; R⁷ and R⁸ independently are —C(R¹⁰)₂—, wherein R¹⁰ independently are —H, a suitable monovalent O-linked moiety, a suitable monovalent C-linked moiety or a suitable halogen, wherein the suitable monovalent O-linked moieties are independently a suitable ester, ether or silyl ether, the monovalent C-linked moieties independently are preferably a suitable optionally substituted alkyl and the suitable halogens independently are preferably fluoro.

In Method B the 3β-hydroxy substituent in a 3β-hydroxy androst-5-ene steroid, represented by structure J is contacted with a tri-substituted phosphine having the structure (R¹⁸)₃P, wherein R¹⁸ independently selected are C₁₋₆ alkyl or aryl, optionally wherein the tri-substituted phosphine is Ph₃P, and a azo-di-carboxylate ester having the structure of R¹⁹OC(O)N═NC(O)OR¹⁹ wherein R¹⁹ are independently selected alkyl, typically C₁₋₆ alkyl, optionally wherein the azo-di-carboxylate ester is diethyl azodicarboxylate (DEAD) or di-isopropyl azodicarboxylate (DIAD) whereby a transient phosphorus-based steroid intermediate is formed. The reaction mixture is subsequently contacted with an organic acid having the structure of R¹²C(O)OH, wherein R¹² is an optionally substituted alkyl or optionally substituted aryl, optionally wherein the organic acid is acetic acid or p-nitrophenyl benzoic acid, capable of reacting with the transient intermediate to provide a 3α-O-linked-androst-5-ene steroid represented by structure K derived a 3α-hydroxy-androst-5-ene steroid precursor; whereby the 3β-hydroxy substituent in J is exchanged with an ester moiety in the α-configuration.

In some embodiments R¹² is an electron withdrawing moiety wherein the electron withdrawing moiety provides an ester in structure K more readily hydrolyzed under basic aqueous condition than acetate. In some embodiments the electron withdrawing moiety is phenyl substituted with one or more electron withdrawing groups selected from the group consisting of bromo, chloro, fluoro and nitro. In a preferred embodiment the electron withdrawing moiety is p-nitrophenyl wherein R¹²C(O)OH is p-nitrophenylbenzoic acid. Hydrolysis of the C-1α-ester in K then provides a 3α-hydroxy androst-5-ene steroid of structure L, wherein R¹ is —OH, thus completing inversion of configuration at position C-1 of a 3β-hydroxy-androst-5-ene steroid to provide a 3α-hydroxy-androst-5-ene steroid. 3α-O-linked-androst-5-en-7-one steroids having the structure M may also be obtained by C7-oxidation of a suitably protected 3α-O-linked-androst-5-ene, obtained or derived from the reaction sequence of Scheme 4, wherein the suitably protected 3α-O-linked-androst-5-ene has structure K, wherein R¹ is a an ester derived from R¹²COOH, or has the structure L wherein R¹ is —OR^{PR} wherein R^{PR} is a protecting group derived from contacting a 3α-hydroxy-androst-5-ene steroid product of Scheme 4 with a suitable electrophile. Methods to affect C-7 oxidation of a 3α-O-linked-androst-5-ene steroid to provide a 3α-O-linked-androst-5-en-7-one are as previously described for obtaining 313-O-linked-androst-5-en-7-one precursors for Method A.

Scheme 4. Preparation of 3α-O-linked steroids by Method B from a 3β-hydroxy-androst-5-ene precursor

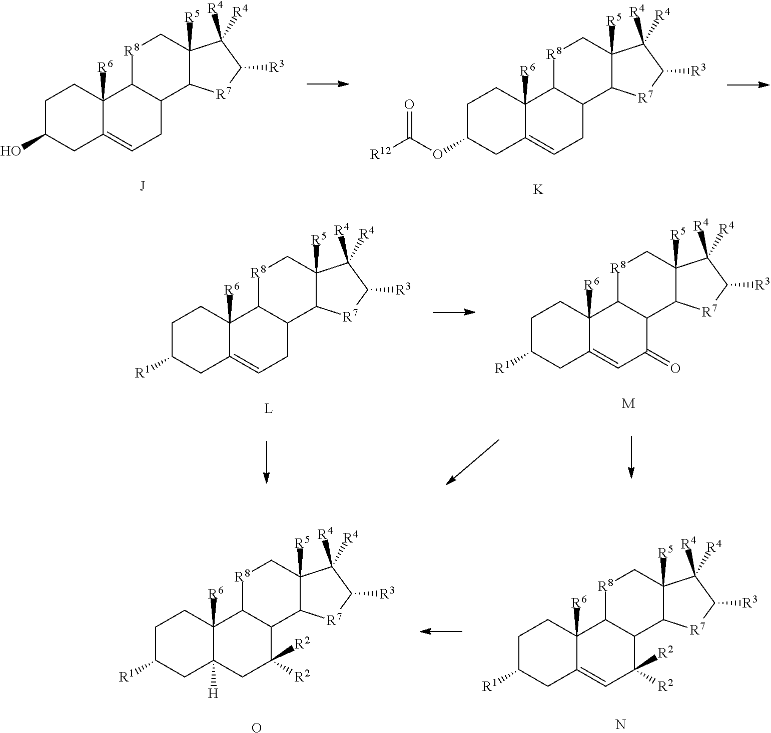

Other 3α-O-linked-androst-5-ene steroids including 3α,713-di-hydroxy-androst-5-ene or 3α,7α-di-hydroxy-androst-5-ene steroids may be prepared from a suitably protected 3α-hydroxy-androst-5-en-7-one steroid of structure M by subsequent contact with a hydrogen donor (a second hydrogen donor) that effects reduction of the 7-one functional group to C7-hydroxy. This second hydrogen donor will provide a 3α,713-di-hydroxy-androst-5-ene steroid product or a 3α,7α-di-hydroxy-androst-5-ene steroid product, represented by structure N, wherein one R² is —OH and the other R² is —H, or a mixture thereof, that may be separated by standard chromatographic methods. A suitably protected 3α-hydroxy-androst-5-en-7-one steroid may also be contacted with a organometallic agent having the structure R²-M, wherein M is a suitable optionally substituted alkyl, alkenyl or alkynyl moiety and M is a Group 1, Group 2, or a transition metal to provide a product of structure M wherein one R² is —OH, typically in the β-configuration and the other R² is derived from the organometallic agent and is typically in the α-configuration.

3α-O-linked-5α-androstane, 3α-O-linked-5α-androstan-7-one and 3α,7ζ-di-O-linked-5α-androstane steroids represented by structure 0, wherein both R² are —H or together are ═O or one R² is a monovalent O-linked moiety and the other R² is —H or a monovalent C-linked moiety, may be prepared from suitably protected 3α-O-linked-androst-5-ene steroids having structure L, M or N through contact with a hydrogen donor capable of saturating the Δ⁵-functional group (a third hydrogen donor) that may or may not reduce other functional groups present in the molecule depending on reaction conditions and protecting group strategy. For example, an androst-5-en-7-one steroid prepared according to the reaction sequence of Scheme 4 may be reduced from contact with a third reducing agent to a 3α-O-linked-74-hydroxy-5α-androstane or a 3α-O-linked-5α-androstan-7-one by complete saturation of the α,β-unsaturated functional group or selective Δ⁵ saturation, respectively.

Steroids with C17-disubstitution having structures L, M, N or O wherein one R⁴ is -a monovalent O-linked moiety and the other R⁴ is a monovalent C-linked moiety such as an optionally substituted alkyl, optionally substituted alkenyl or optionally substituted alkynyl may be prepared either by using a 3β-hydroxy-androst-5-ene steroid already containing C17-disubstitution as a precursor for the reaction sequence of Scheme 4 or by contacting a suitably protected androst-5-en-17-one of structure L, M or N or a suitably protected 5α-androstane-17-one of structure O wherein in L, M, N or O R⁴ together are ═O with a organometallic reagent having the structure R⁴-M, wherein R⁴ is a suitable optionally substituted alkyl, alkenyl or alkynyl moiety and M is a Group 1, Group 2, or a transition metal and optionally quenching the reaction between the androst-5-en-17-one steroid and the organometallic reagent with an electrophile. The C17-disubstituted steroid thus formed has one R⁴ as —OH, or another monovalent O-linked moiety provided by the quenching electrophile, and the other R⁴ derived from the organometallic agent.

In the structures of Scheme 4, R³ is —H, a suitable halogen, optionally chloro or fluoro, a suitable monovalent C-linked moiety or a suitable monovalent O-linked moiety, one R⁴ is a suitable monovalent O-linked moiety and the other R⁴ is —H, a suitable monovalent O-linked moiety or a suitable monovalent C-linked moiety, optionally wherein the monovalent C-linked moieties independently are a suitable optionally substituted alkyl, optionally substituted alkenyl or optionally substituted alkynyl, optionally C₁₋₆ alkyl, C₂₋₆ alkenyl or C₂₋₆ alkynyl, or both R⁴ together are ═O or divalent O-linked moiety that defines a cyclic ketal, optionally having the structure of —O—[C(R₁₆)]ₙ—O—, wherein n=2 or 3 and R¹⁶ independently are —H or C₁₋₄ alkyl; R⁵ and R⁶ are —H or independently selected optionally substituted alkyl, optionally —CH₃ or CH₂OR^{PR}; R⁷, R⁸ independently are —C(R¹⁰)₂—, wherein R¹⁰ independently are —H, a suitable halogen, optionally bromo, chloro or fluoro, a suitable monovalent C-linked moiety, optionally a C₁₋₆ alkyl, or a suitable monovalent O-linked moiety; wherein the monovalent O-linked moieties independently are an ester, optionally a C₁₋₆ ester, an ether, optionally a C₁₋₆ ether, silyl ether, optionally —OSi(R¹³)₃, or —OR^{PR}, wherein R¹³ independently are alkyl or aryl, optionally C₁₋₄ alkyl or phenyl and R^{PR} independently are a protecting group.

In some embodiments of Method B, 3α-hydroxy-androst-5-en-17-one steroids of structure 12 are prepared according to Scheme 5, wherein R³ is —H, a suitable halogen, a suitable monovalent O-linked moiety or a suitable monovalent C-linked moiety and R⁸ is —C(R¹⁰)₂ wherein R¹⁰ independently are —H, a suitable monovalent O-linked moiety, a suitable monovalent C-linked moiety or a suitable halogen, wherein the suitable halogens independently are preferably bromo, chloro or fluoro, the suitable monovalent O-linked moieties independently are a suitable ester, ether or silyl ether and the suitable monovalent C-linked moieties independently are preferably a suitable optionally substituted alkyl. In a particular example 12a, (i.e., 3α-hydroxy-androst-5-en-17-one or 3α-DHEA), was prepared from 3β-hydroxy-androst-5-ene 10a using Method B according the reaction sequence of Scheme 5, wherein R³ is —H and R⁸ is —CH₂—.

Scheme 5. Preparation of 3α-hydroxy-androst-5-ene steroids by Method B from a 3β-hydroxy-androst-5-ene precursor

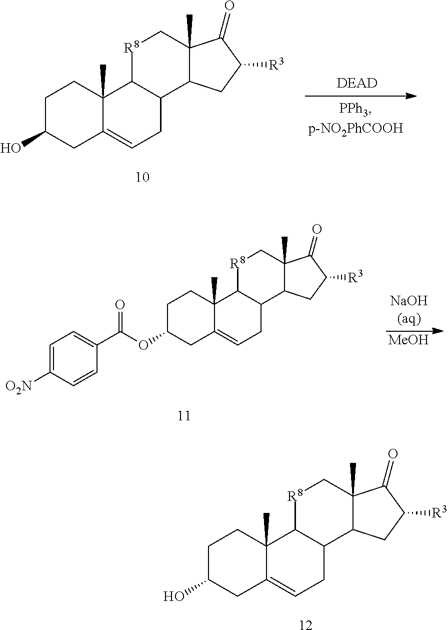

Further 3α-hydroxy-androst-5-ene steroids are prepared according to Scheme 5-1 from steroid products of Scheme 5 by suitable protection of the ═O moiety at position C-17 and the 3α-hydroxy substituent of 12 followed by oxidation at position C-7 to provide, after deprotection, androst-5-en-7,17-dione-3α-ol steroids. Additionally, the C-7 ketone of the 3α-hydroxy-androst-5-ene steroid so formed may be reduced by contact, after suitable protection with a second hydrogen donor, wherein the second hydrogen donor is a hydride donor, to provide, after deprotection, androst-5-en-17-one-3α,7α-diol steroids and androst-5-en-17-one-3α,713-diol steroids. Androst-5-en-7,17-dione-3α-ol, androst-5-en-17-one-3α,713-diol, androst-5-en-17-one-3α,7α-diol and 3α-DHEA that are prepared in this manner may also be used as intermediates for preparing other biologically active 3α-hydroxy steroids.

Additional 3α-hydroxy-androst-5-ene steroids are prepared from steroid products of Scheme 5 having di-substitution at C-17 according to the reaction sequence of Scheme 5-1, wherein R⁴ in the β-configuration is a monovalent O-linked moiety and R⁴ in the α-configuration is optionally substituted alkyl, optionally substituted alkenyl or optionally substituted alkynyl may be effected by contacting a suitably protected 3α-hydroxy steroid prepared or derived from the reaction product of Scheme 5 and having a ═O moiety at position C-17, such as a suitably protected androst-5-en-7,17-dione-3α-ol, androst-5-en-17-one-3α,7α-diol, androst-5-en-17-one-3α,7β-diol or 3α-DHEA, with an suitable organometallic agent.

Scheme 5-1. Preparation of 3α-hydroxy-androst-5-ene steroids having a monovalent O-linked moiety at position C-7 and-or having di-substitution at C-17 from steroid precursors obtained from Method B

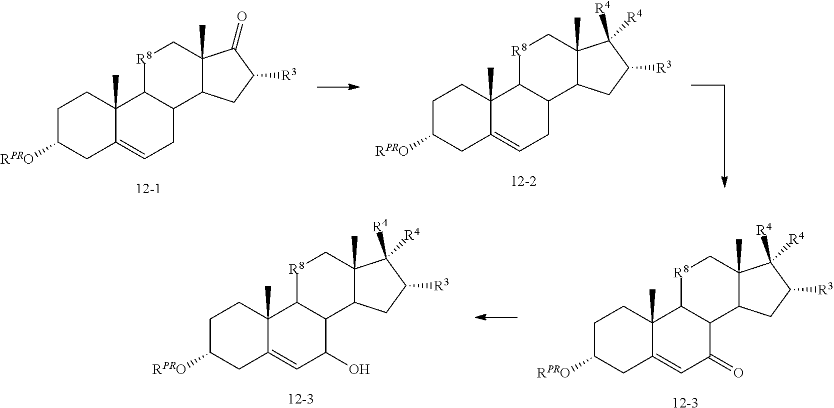

Procedures to prepare 3α-hydroxy-androst-5-ene steroids having disubstitution at C-17, wherein one C-17 substituent in the β-configuration is —OH and the other C-17 substituent in the α-configuration is —C≡CH form 3α-hydroxy steroid prepared according to the reaction sequence of Scheme 5 include for example contacting a suitably protected 3α-hydroxy steroid precursors having a ═O moiety at position C-17 with sodium acetylide, lithium acetylide (as its ethylene diamine complex), ethynyl magnesium halide (e.g., chloride or bromide) or ethynyl zinc halide, as for example in U.S. Pat. No. 2,243,88 (specifically incorporated by reference herein), in diethylether or other ether solvents such as tetrahydrofuran, 1,2-dimethoxyethane, 2-methoxyethylether and the like.

5α-Androstane steroids may be obtained from the androst-5-ene steroids prepared from the reaction sequences of Scheme 5 or Scheme 5-1 by contacting these steroids having suitable protection with a third hydrogen donor such as a hydrogen atom donor wherein the Δ⁵ functional group is reduced whereby a 5α-androstane steroid is produced.

Numbered embodiments. The following embodiments exemplify one or more aspects of the invention are not meant to be limiting in any way.

1. A process to prepare a 3α-O-linked steroid comprising the steps of (1) contacting a protected 3α,4α-epoxyandrost-5-ene having the structure

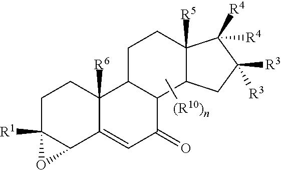

wherein R¹ is —H or a suitable optionally substituted alkyl;

R³ independently are —H, a suitable halogen, a suitable monovalent O-linked moiety, or a suitable monovalent C-linked moiety;

R⁴ independently are a suitable monovalent O-linked moiety or both of R⁴ together are —OC(R¹⁶)₂C(R¹⁶)₂O— or —OC(R¹⁶)₂C(R¹⁶)₂C(R¹⁶)₂O—, wherein R¹⁶ independently are optionally substituted alkyl or two of R¹⁶ and the carbon(s) to which they are attached comprise a cycloalkyl and the remaining R¹⁶ are independently optionally substituted alkyl;

R⁵ and R⁶ independently are —H or a suitable optionally substituted alkyl; (R¹⁰)ₙ—, is 0, 1, 2, 3 or 4 independently selected R¹⁰ substituents attached to the steroid ring replacing hydrogen other than at positions C-3, C-7, C-16 and C-17; wherein R¹⁰ substituents replace none, one, two, three or four positions selected from the group consisting of positions C-1, C-2, C-4, C-6, C-9, C-11, C-12 and C-15, wherein none, one or two R¹⁰ may be present at positions C-1, C-2, C-11 and C-15 and none or one R¹⁰ may be present at positions C-4, C-6 or C-9, wherein R¹⁰, if present at position C-9 is —Cl or —F, if present at positions C-4 or C-6 is independently selected optionally substituted alkyl and if present at positions C-1, C-2, C-11 or C-15 is independently selected halogen, suitable monovalent C-linked moiety or suitable monovalent O-linked moiety; optionally wherein the suitable halogens independently are chloro or fluoro, the suitable monovalent O-linked moieties independently are —OH, a suitable —OR^{PR}, ester or ether and the suitable monovalent C-linked moieties are suitable optionally substituted alkyl wherein R^{PR} independently are a protecting group,

with a first hydrogen donor wherein the 3α,4α epoxy functional group is preferentially reduced relative to the Δ⁵ functional group and wherein reduction of the 3α,4α epoxy functional group occurs preferentially at position C-4 with retention of configuration at position C-3 with or without concomitant C-7 ketone reduction, wherein the first hydrogen donor optionally is an aluminum hydride or a palladium metal catalyst in the presence of hydrogen gas; and optionally (2) contacting the product of step 1 with an electrophile wherein a monovalent O-linked moiety is formed at position C-3 or at positions C-3 and C-7 wherein the monovalent O-linked moiety(ies) are derived from the electrophile; whereby a 3α-O-linked-androst-5-en-7-one steroid is prepared or a 3α,7ζ-di-O-linked-androst-5-ene steroid is prepared after protecting group removal.

2. The process of embodiment 1, wherein the 3α-O-linked steroid prepared has the structure

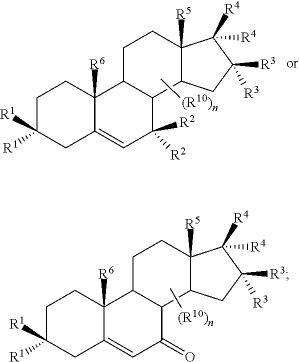

wherein R¹ in the β-configuration is —H; R¹ in the α-configuration is a monovalent O-linked moiety, optionally —OH;

one R² is a monovalent O-linked moiety, optionally —OH or a C₂₋₄ ester or a C₁₋₄ ether such as —OC(O)CH₃, —OCH₃ or —OC₂H₅, and the other R² is —H;

R³ independently are —H, halogen, a monovalent O-linked moiety, optionally —OH or a C₂₋₄ ester or a C₁₋₄ether such as —OC(O)CH₃, —OCH₃ or —OC₂H₅, or a monovalent C-linked moiety, optionally a C₁₋₄ alkyl such as —CH₃, —C₂H₅ or —CH₂CH₂CH₃;

R⁴ independently are a monovalent O-linked moiety or both of R⁴ together are ═O, —OC(R¹⁶)₂C(R¹⁶)₂O—, or —OC(R¹⁶)₂C(R¹⁶)₂C(R¹⁶)₂O—, wherein R¹⁶ independently are C₁₋₄ alkyl or two of R¹⁶ and the carbon(s) to which they are attached comprise a cycloalkyl and the remaining R¹⁶ are independently C₁₋₄ alkyl;

R⁵ and R⁶ independently are —H, —CH₃ or —CH₂OH, optionally wherein (i) R⁵ and R⁶ are both —CH₃, (ii) R⁵ is —CH₂OH and R⁶ is —CH₃ or (iii) R⁵ is —CH₃ and R⁶ is —H and

(R¹⁰)ₙ is 0, 1 or 2 independently selected R¹⁰ substituents attached to the steroid ring replacing hydrogen other than at positions C-3, C-7, C-16 and C-17, wherein R¹⁰ substituents replace none, one, two, three or four positions selected from the group consisting of positions C-1, C-2, C-9, C-11, C-12 and C-15, wherein none, one or two R¹⁰ may be present at positions C-1, C-2, C-11 and C-15 and, wherein R¹⁰, if present at position C-9 is —Cl or —F and if present at positions C-1, C-2, C-11 or C-15 is an independently selected monovalent C-linked moiety or a monovalent O-linked moiety; wherein the halogens independently are chloro or fluoro, the monovalent O-linked moieties independently are —OH, —OR^{PR}, wherein R^{PR} is a protecting group, an ester, an ether or a silylether and the monovalent C-linked moieties independently are alkyl.

3. The process of embodiment 1 further comprising the step(s) of contacting a 3α-O-linked-androst-5-en-7-one prepared from step 1 or step 2 with a second hydrogen donor to reduce the C-7 ketone or contacting a 3α,7ζ-di-O-linked-androst-5-ene steroid prepared from step 1 or step 2 with a third hydrogen donor to reduce the Δ⁵ functional group reduction or sequentially contacting the 3α-O-linked-androst-5-en-7-one prepared from step 1 or step 2 with a second and third hydrogen donor, whereby a 3α-O-linked-5α-androstan-7-one is prepared or a 3α,7ζ-di-O-linked-5α-androstane steroid is prepared after protecting group removal.

4. The process of embodiment 3, wherein the 3α-O-linked steroid prepared has the structure

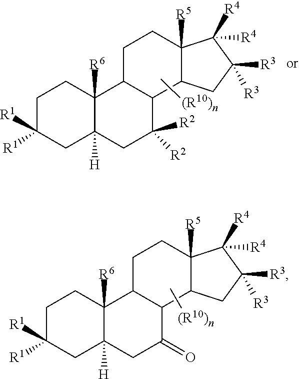

wherein R¹ in the β-configuration is —H; R¹ in the α-configuration is a monovalent O-linked moiety; one R² is a monovalent O-linked moiety, optionally —OH, an ester or an ether such as methyl ether or acetate, and the other R² is —H;

R³ independently are —H, halogen, a monovalent O-linked moiety, optionally —OH or a C₂₋₄ ester or a C₁₋₄ether such as —OC(O)CH₃, —OCH₃ or —OC₂H₅, or a monovalent C-linked moiety, optionally a C₁₋₄ alkyl such as —CH₃, —C₂H₅ or —CH₂CH₂CH₃;

R⁴ independently are a monovalent O-linked moiety or both of R⁴ together are ═O or —OC(R¹⁶)₂C(R¹⁶)₂O—, wherein R¹⁶ independently are C₁₋₄ alkyl or two of R¹⁶ and the carbon(s) to which they are attached comprise a cycloalkyl and the remaining R¹⁶ are independently C₁₋₄ alkyl; R⁵ and R⁶ independently are —H or optionally substituted alkyl, optionally wherein (i) R⁵ and R⁶ are both —CH₃, (ii) R⁵ is —CH₂OH and R⁶ is —CH₃ or (iii) R⁵ is —CH₃ and R⁶ is —H;

(R¹⁰)ₙ is 0, 1 or 2 independently selected R¹⁰ substituents attached to the steroid ring replacing hydrogen other than at positions C-3, C-7, C-16 and C-17, wherein R¹⁰ substituents replace none, one, two, three or four positions selected from the group consisting of positions C-1, C-2, C-9, C-11, C-12 and C-15, wherein none, one or two R¹⁰ may be present at positions C-1, C-2, C-11 and C-15 and, wherein R¹⁰, if present at position C-9 is —Cl or —F and if present at positions C-1, C-2, C-11 or C-15 is an independently selected monovalent C-linked moiety or a monovalent O-linked moiety;

wherein the halogens independently are chloro or fluoro, the monovalent O-linked moieties independently are —OH, —OR^{PR}, wherein R^{PR} is a protecting group, an ester, an ether or a silyl ether and the monovalent C-linked moieties independently are alkyl.

5. The process of embodiment 2 or 4 wherein the monovalent O-linked moieties of R¹ and R² independently are —OH, —OR^{PR}, wherein R^{PR} is a protecting group, an ester or a silyl ether; and one R³ is —H, or halogen, wherein the halogen is chloro or fluoro, a monovalent O-linked moiety, wherein the monovalent O-linked moiety is —OH, —OR^{PR}, an ester, an ether or a silyl ether, or a monovalent C-linked moiety, wherein the monovalent C-linked moiety is alkyl; and the other R³ is —H; wherein the silyl ethers independently selected have the formula —OSi(R¹³)₃, wherein R¹³ independently are alkyl or aryl;

6. The process of embodiment 1 wherein one of R⁴ is a monovalent O-linked moiety, wherein the monovalent O-linked moiety is —OR^{PR}, an ester, an ether or a silyl ether having the formula —OSi(R¹³)₃, wherein R¹³ independently are alkyl or aryl and the other R⁴ is —H or a monovalent O-linked moiety, wherein the monovalent O-linked moiety is an ester or an ether, or both R⁴ together are —OCH₂CH₂O—, optionally wherein R^{PR} are acetyl or trimethylsilyl and —OR^{PR} are acetate or trimethylsilyl ether.

7. The process of embodiment 1 or 3 additionally comprising the step of contacting a 3α,7ζ-di-O-linked-androst-5-ene steroid or a 3α,7ζ-di-O-linked-5α-androstane steroid product so obtained with an organometallic agent having the formula R⁴-M, wherein M is a Group I, Group II or transition metal, optionally Na, Li, Mg or Zn, optionally wherein the steroid product so obtained has the structure

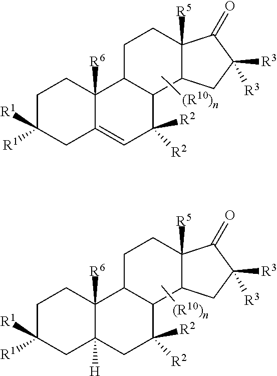

wherein R¹ in the β-configuration is —H, R¹ in the α-configuration is —OR^{PR}, an ether or a silyl ether; one R² is —OR^{PR}, an ether or a silyl ether and the other R² is —H; R³ independently are —H, an ether, a silyl ether, chloro or fluoro;

R⁵ and R⁶ independently are —H or optionally substituted alkyl wherein the optionally substituted alkyl independently are —CH₃, —CH₂(ether) or —CH₂(silyl ether);

(R¹⁰)ₙ is 0, 1 or 2 independently selected R¹⁰ substituents attached to the steroid ring replacing hydrogen other than at positions C-3, C-7, C-16 and C-17, wherein R¹⁰ substituents replace none, one, two, three or four positions selected from the group consisting of positions C-1, C-2, C-9, C-11, C-12 and C-15, wherein none, one or two R¹⁰ may be present at positions C-1, C-2, C-11 and C-15 and, wherein R¹⁰, if present at position C-9 is —Cl or —F and if present at positions C-1, C-2, C-11 or C-15 is an independently selected alkyl, —OR^{PR}, an ether or a silyl ether; and R^{PR} independently are a protecting group and the silyl ethers independently selected have the formula —OSi(R¹³)₃ wherein R¹³ independently are alkyl or aryl.

8. The process of embodiment 5 wherein, in each independently selected —OSi(R¹³)₃, three of R¹³ are —CH₃ or —CH₂CH₃ or one of R¹³ is phenyl or t-butyl (t-Bu) and the remaining R¹³ are independently —CH₃ or —CH₂CH₃, each independently selected ester is —OC(O)CH₃, —OC(O)CH₂CH₃ or —OC(O)Ph and the monovalent C-linked moiety of R⁴ is optionally substituted C₁₋₄ alkyl, optionally —CH₃ or —CH₂CH₃, optionally substituted C₂₋₄ alkenyl, optionally —CH═CH₂ or optionally substituted C₃₋₄ alkynyl, optionally —C≡CCH₃, —C≡CH or —C≡C≡Cl.

9. The process of embodiment 8 wherein —OSi(R¹³)₃ is —OSi(CH₃)₃ or —OSi(t-Bu)(CH₃)₂, the ester is —OC(O)CH₃ (acetate) and the monovalent C-linked moiety of R⁴ is —CH₃, —CH₂CH₃, —CH═CH₂ or —C≡CH.

10. The process of embodiment 2, 4 or 7 wherein the monovalent O-linked moiety of R¹ in the α-configuration is —OH or an ester;

one R² is a monovalent O-linked moiety wherein the monovalent O-linked moiety is —OH and the other R² is —H;

one of R³ is —H and the other R³ is —H or a monovalent O-linked moiety, wherein the monovalent O-linked moiety is —OH;

one of R⁴ is an O-linked moiety, wherein the monovalent O-linked moiety is —OH and the other R⁴ is —H or a monovalent C-linked moiety, wherein the monovalent C-linked moiety is optionally substituted alkyl, optionally substituted alkenyl or optionally substituted alkynyl;

R⁵ is —CH₃ or —CH₂OH; R⁶ is —H, —CH₃ or —CH₂OH;

(R¹⁰)ₙ is 0, 1 or 2 independently selected R¹⁰ substituents attached to the steroid ring replacing hydrogen other than at positions C-3, C-7, C-16 and C-17, wherein R¹⁰ substituents are at none, one or two selected from the group consisting of positions C-1, C-2, C-11, and C-15, wherein none, one or two R¹⁰ may be present at positions C-1, C-2, C-11 and C-15 and, wherein R¹⁰, if present at position C-1, C-2, C-11 or C-15 is an independently selected monovalent C-linked moiety or monovalent O-linked moiety, wherein the monovalent O-linked moiety is —OH, —OR^{PR}, wherein R^{PR} is a protecting group, an ester, an ether or a silyl ether and the monovalent C-linked moiety is alkyl.

11. The process of embodiment 10 wherein the 3α-O-linked steroid prepared has the structure

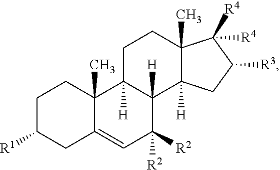

wherein R² independently or together are —H, —OH, an ester, —OR^{PR} or ═O (ketone), provided that R² are not both —OH; and/or

R³ is —H, C₁₋₆ alkyl, halogen, —OH, C₁₋₆ ester, —OR^{PR} or C₁₋₆ ether, optionally wherein the halogen is fluoro, the ester is acetate or n-propionate, the ether is methoxy or ethoxy the alkyl is methyl, ethyl, n-propyl or iso-propyl and —OR^{PR} is trimethylsilyloxy or t-butyldimethylsilyloxy.

12. The process of embodiment 10 wherein the 3α-O-linked steroid prepared has the structure

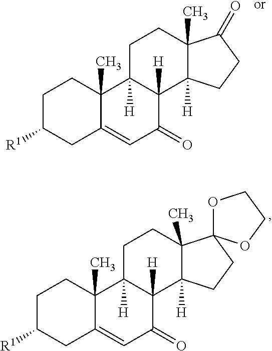

wherein R¹ is —OH or a C₁₋₆ ester, optionally acetate.

13. The process of embodiment 10 wherein the 3α-O-linked steroid prepared has the structure

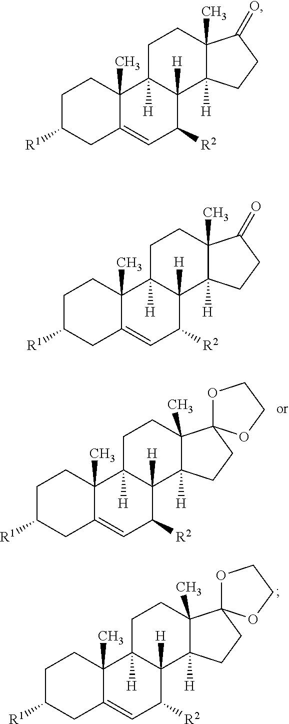

wherein the 3α-O-linked steroid prepared has the structure wherein R¹ and R² independently are —OH or a C₁₋₆ ester, optionally acetate.

14. The process of embodiment 10 wherein the 3α-O-linked steroid prepared has the structure

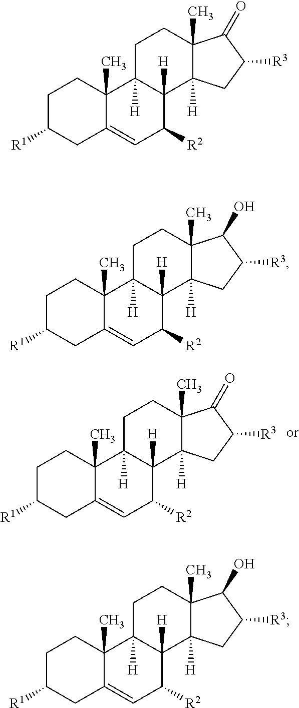

wherein R¹ is —OH or a C₁₋₆ ester; R² is —H, —OH or a C₁₋₆ ester; R³ is —OH, halogen or a C₁₋₆ ester; and optionally wherein halogen is —Br or —F or optionally wherein one or more of the C₁₋₆ esters are acetate, or an analog on any of the foregoing structures wherein (i) R³ is in the β-configuration or (ii) —OH at the 17-position is in the α-configuration.

15. The process of embodiment 10 wherein the 3α-O-linked steroid prepared has the structure

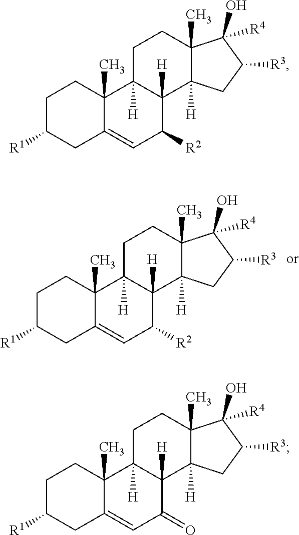

wherein R¹ is —OH or a C₁₋₆ ester; R² and R³ independently are —H, —OH or a C₁₋₆ ester; R⁴ is a monovalent C-linked moiety, wherein the monovalent C-linked moiety is optionally substituted C₁₋₆ alkyl, optionally substituted C₂₋₆ alkenyl or optionally substituted C₂₋₆ alkynyl, or an analog on any of the foregoing structures wherein (i) R³ is in the β-configuration or (ii) —OH at the 17-position is in the α-configuration and R⁴ at the 17-position is in the β-configuration; and optionally wherein the monovalent C-linked moiety of R⁴ is —CH₃, —CH═CH₂ or —C≡CH or optionally wherein one or more of the C₁₋₆ esters are acetate.

16. The process of embodiment 10 wherein the 3α-O-linked steroid prepared has the structure

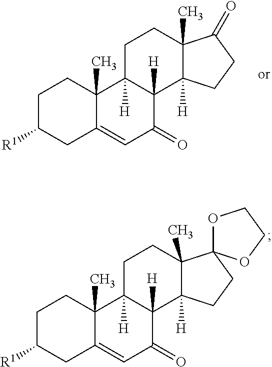

wherein R¹ is —OH or a C₁₋₆ ester.

17. The process of embodiment 1 or 2 wherein the 3α-O-linked steroid prepared is androst-5-en-7,17-dione-3α-ol, 3α-acetoxy-androst-5-en-7,17-dione, 17,17-ethylenedioxy-androst-5-en-7-one-3α-ol or 17,17-ethylenedioxy-3α-acetoxy androst-5-en-7-one.

18. The process of embodiment 1 or 3 wherein the 3α-O-linked steroid prepared is androst-5-en-17-one-3α,76-diol, 3α-acetoxy-androst-5-en-17-one-76-ol, androst-5-en-17-one-3α,7α-diol, 3α-acetoxy-androst-5-en-17-one-7α-ol, 17,17-ethylenedioxy-androst-5-ene-3α,76-diol, 17,17 ethylenedioxy-3α-acetoxy-androst-5-ene-76-ol, 17,17-ethylenedioxy-androst-5-ene-3α,7α-diol or 17,17-ethylenedioxy-3α-acetoxy-androst-5-ene-7α-ol.

19. The process of embodiment 1 or 3 wherein the 3α-O-linked steroid prepared is androst-5-en-17-one-3α,76,16α-triol; 16α-methoxy-androst-5-en-17-one-3α,76-diol, 16α-fluoro-androst-5-en-17-one-3α,76-diol, androst-5-ene-3α,76,16α,17β-tetrol; 16α-methoxy-androst-5-ene-3α,7β,17β-triol, 16α-fluoro-androst-5-ene-3α,7β,17β-triol, androst-5-en-17-one-3α,7α,16α-triol; 16α-methoxy-androst-5-en-17-one-3α,7α-diol, 16α-fluoro-androst-5-en-17-one-3α,7α-diol, androst-5-ene-3α,7α,16α,17β-tetrol; 16α-methoxy-androst-5-ene-3α,7α,17β-triol or 16α-fluoro-androst-5-ene-3α,7α,17β-triol.

20. The process of embodiment 7 wherein the 3α-O-linked steroid prepared is 17α-ethynyl-androst-5-ene-3α,7β,17β-triol, 17α-ethenyl-androst-5-ene-3α,7β,17β-triol, 17α-ethyl-androst-5-ene-3α,7β,17β-triol, 17α-methyl-androst-5-ene-3α,7β,17β-triol, 17α-ethynyl-androst-5-ene-3α,7α,17β-triol, 17α-ethynyl-androst-5-ene-3α,76,16α,17β-tetrol, 17α-ethynyl-16α-fluoro-androst-5-ene-3α,7β,17β-triol, 17α-ethynyl-androst-5-en-7-one-3α,176-diol or 17α-ethynyl-androst-5-en-7-one-3α,16α,17β-triol.

21. The process of embodiment 7 wherein the 3α-O-linked steroid prepared is 17α-ethynyl-5α-androstane-3α,7β,17β-triol, 17α-ethenyl-5α-androst-5-ene-3α,7β,17β-triol, 17α-ethyl-5α-androstane-3α,7β,17β-triol, 17α-methyl-5α-androstane-3α,7β,17β-triol, 17α-ethynyl-5α-androstane-3α,7α,17β-triol, 17α-ethynyl-5α-androstane-3α,76,16α,17β-tetrol, 17α-ethynyl-16α-fluoro-5α-androstane-3α,7β,17β-triol, 17α-ethynyl-5α-androstan-7-one-3α,17β-diol or 17α-ethynyl-5α-androstan-7-one-3α,16α,17β-triol.

22. The process of embodiment 3 wherein the 3α-O-linked steroid prepared is 5α-androstane-17-one-3α,76-diol, 3α-acetoxy-5α-androstan-17-one-76-ol, 5α-androstan-17-one-3α,7α-diol, 3α-acetoxy-5α-androstan-17-one-7α-ol, 17,17-ethylenedioxy-5α-androstane-3α,76-diol, 17,17 ethylenedioxy-3α-acetoxy-5α-androstane-76-ol, 17,17-ethylenedioxy-5α-androstane-3α,7α-diol or 17,17-ethylenedioxy-3α-acetoxy-5α-androstane-7α-ol.

23. The process of embodiment 3 wherein the 3α-O-linked steroid prepared is 5α-androstan-17-one-3α,713,16α-triol; 16α-methoxy-5α-androstan-17-one-3α,713-diol, 16α-fluoro-5α-androstan-17-one-3α,713-diol, 5α-androstane-3α,7β,16α,17β-tetrol; 16α-methoxy-5α-androstane-3α,7β,17β-triol, 16α-fluoro-5α-androstane-3α,7β,17β-triol, 5α-androstan-17-one-3α,7α,16α-triol, 16α-methoxy-5α-androstan-17-one-3α,7α-diol, 16α-fluoro-5α-androstane-17-one-3α,7α-diol, 5α-androstane-3α,7α,16α,17β-tetrol; 16α-methoxy-5α-androstane-3α,7α,17β-triol or 16α-fluoro-5α-androstane-3α,7α,17β-triol.

24. A process to prepare a 3α-O-linked androst-5-ene steroid comprising the steps of (1) contacting a suitably protected 3α,4α-epoxy-androst-5-ene with a first hydrogen donor wherein the 3α,4α epoxy functional group is preferentially reduced relative to the Δ⁵ functional group and wherein reduction of the 3α,4α epoxy functional group occurs preferentially at position C-4 with retention of configuration at position C-3 wherein the suitably protected 3α,4α-epoxy-androst-5-ene has the structure

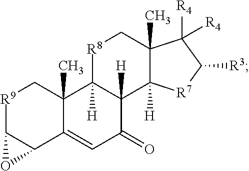

wherein R³ is —H, a suitable halogen, a suitable monovalent O-linked moiety or a suitable monovalent C-linked moiety; and R⁴ independently are an ether or both R⁴ together are —OC(R¹⁶)₂C(R¹⁶)₂O—, wherein R¹⁶ independently are —H or C₁₋₄ alkyl or two of R¹⁶ and the carbon(s) to which they are attached form a cycloalkyl, optionally a C₃, C₅ or C₆ cycloalkyl, and the remaining R¹⁶ are —H; R⁹, R⁷ and R⁸ independently are —C(R¹⁰)₂, wherein R¹⁰ independently are —H or a suitable monovalent O-linked moiety, whereby a 3α-O-linked androst-5-ene product having a ═O (ketone) moiety at position C-7 is obtained

(2) optionally contacting the product of step 1 with an electrophile wherein a monovalent O-linked group is formed at position 3, wherein the monovalent O-linked group is other than —OH.

25. The process of embodiment 24 further comprising the step of (1) contacting a suitably protected 3α-O-linked androst-5-en-7-one obtained or prepared from the 3α-O-linked androst-5-ene product of claim 20 with a second hydrogen donor wherein the suitably protected 3α-O-linked androst-5-en-7-one has the structure

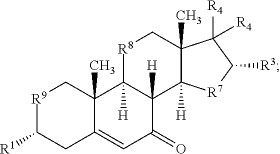

wherein R¹ is a suitable monovalent O-linked moiety; R³ is —H, a suitable C-linked moiety, a suitable halogen or a suitable monovalent O-linked moiety;

R⁴ independently are an ether or one R⁴ is a suitable monovalent O-linked moiety and the other R⁴ is —H or both R⁴ together are ═O (ketone) or —OC(R¹⁶)₂C(R¹⁶)₂O— (ketal) wherein R¹⁶ independently are —H or C₁₋₄ alkyl or two of R¹⁶ and the carbon(s) to which they are attached form a cycloalkyl, optionally a C₃, C₅ or C₆ cycloalkyl, and the remaining R¹⁶ are —H; whereby a 3α-O-linked androst-5-ene product having a monovalent O-linked moiety at position C-7 is obtained wherein the monovalent O-linked moiety is —OH in the α- or β-configuration; optionally wherein the suitable monovalent O-linked moieties are —OR^{PR}, independently selected, wherein R^{PR} is —H or a protecting group; and

(2) optionally contacting the product resulting from step 1 with an electrophile having the structure R¹¹-LG, R¹²—C(O)-LG, LG, (R¹³)₃Si-LG or (R¹⁴)₂N—C(O)-LG wherein LG is a leaving group and R¹¹, R¹², R¹³ and R¹⁴ are a suitable monovalent C-linked moiety; whereby a 3α-O-linked androst-5-ene product having a monovalent O-linked moiety at position C-7 is obtained and the monovalent O-linked moiety is an ether, an ester, a silyl ether or a carbamate.

26. The process of embodiment 25 wherein the suitably protected 3α-O-linked androst-5-en-7-one has the structure

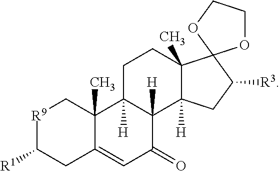

27. The process of embodiment 25 wherein the first hydrogen donor is provided by Pd(0)/H₂, optionally wherein the palladium catalyst is on a support.

28. The process of embodiment 25 wherein the first hydrogen donor is provided by Pd(0)/H₂, wherein the palladium catalyst is supported on carbon black and is suspended in an alcohol-based solvent in the presence of a carbonate salt to which is applied a hydrogenation temperature of between about ambient or about 40° C. or about 22° C. to about 40° C. and a hydrogenation pressure of between about 15.5 psi to about 50 psi H₂, whereby the 3α,4α-epoxy functionality is reduced preferentially and whereby reduction of the 3α,4α epoxy functional group occurs preferentially at position C-4 with retention of configuration at position C-3.

29. The process of embodiment 25 wherein the hydrogenation temperature is ambient or about 22° C., the hydrogenation pressure is about 22 psi H₂, the carbonate salt is potassium carbonate and the alcohol-based solvent is a mixture of ethanol and ethyl acetate in about 5:1 ratio.

30. The process of embodiment 25 wherein the second hydrogen donor is a hydride reducing agent, optionally NaBH₄.

31. The process of embodiment 25 wherein the suitable monovalent O-linked moieties are an ether, —OSi(R¹³)₃, or —OR^{PR}, wherein R^{PR} is —H, a protecting group and R¹³ independently are C₁₋₄ alkyl or aryl, the suitable halogen in R³ is fluoro; and the suitable monovalent C-linked moiety is optionally substituted alkyl, suitably protected.

32. The process of embodiment 31 wherein the suitably protected 3α,4α-epoxy-androst-5-ene is 17,17-ethylenedioxy-3α,4α-epoxy-androst-5-en-7-one, 17,17-di-methoxy-3α,4α-epoxy-androst-5-en-7-one, 17,17-di-ethoxy-3α,4α-epoxy-androst-5-en-7-one, 17,17-(propylene-1,3-dioxy)-3α,4α-epoxy-androst-5-en-7-one, 17,17-tetramethyl-ethylenedioxy-3α,4α-epoxy-androst-5-en-7-one, 17,17-(cyclohex-1,2-yl)-dioxy-3α,4α-epoxy-androst-5-en-7-one, 17,17-ethylenedioxy-16α-methoxy-3α,4α-epoxy-androst-5-en-7-one, 17,17-ethylenedioxy-16α-fluoro-3α,4α-epoxy-androst-5-en-7-one, 17,17-ethylenedioxy-16α-trimethylsilyloxy-3α,4α-epoxy-androst-5-en-7-one or 17,17-ethylenedioxy-16α-(t-butyl-dimethylsilyl)oxy-3α,4α-epoxy-androst-5-en-7-one.

33. A process to prepare a 3α-O-linked-5α-androstane steroid comprising contacting a suitably protected 3α-O-linked androst-5-ene prepared or obtained from the 3α-O-linked androst-5-ene product of claim 24 or 25 with a third hydrogen donor to reduce the Δ⁵ functional group whereby a 3α-O-linked-5α-androstane product is obtained.

34. The process of embodiment 33 wherein the 3α-O-linked-5α-androstane steroid prepared, optionally after protecting group removal, has the structure

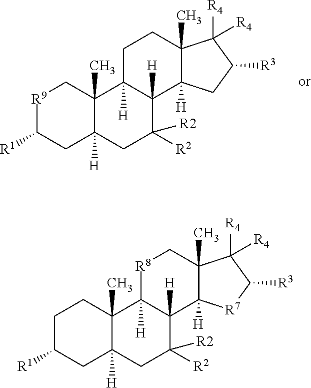

wherein R¹ is —OH, —OR¹¹, —OC(O)—R¹² or —OSi(R¹³)₃; one of R² is —OH, —OR¹¹, —OC(O) —R¹² or —OSi(R¹³)₃ and the other R² is —H or both R² together are ═O; R³ is —H, —OH, —OR¹¹, —OC(O)—R¹²—OSi(R¹³)₃, halogen or C₁₋₄ alkyl; R⁴ independently or together are —OH, —OR¹¹, —OC(O)—R¹², —OSi(R¹³)₃, ═O or —OC(R¹⁶)₂C(R¹⁶)₂O—; R⁷ and R⁸ independently are —C(R¹⁰)₂— wherein both R¹⁰ are —H or one R¹⁰ is α-OH—, β-OH, α-ester, or β-ester and the other R¹⁰ is —H; R⁹ is —C(R¹⁰)₂—, wherein one R¹⁰ is α-OH, β-OH, α-ester or β-ester and the other R¹⁰ is —H; optionally wherein R⁹ is —CH(α-OH)—; optionally wherein (i) R⁷ and R⁸ are —CH₂—, (ii) R⁷ is —CH(α-OH)— or —CH(β-OH)— and R⁸ is —CH₂— or (iii) R⁷ is —CH₂— and R⁸ is —CH(β-OH)—;

R¹¹, R¹² and R¹³ independently are optionally substituted C₁₋₆ alkyl or optionally substituted aryl; and R¹⁶ independently are —H or C₁₋₄ alkyl or two of R¹⁶ and the carbon(s) to which they are attached form a cycloalkyl, optionally C₃, C₅ or C₆ cycloalkyl, and the remaining R¹⁶ are —H; and

optionally wherein the optionally substituted C₁₋₆ alkyl of each R¹¹, independently selected, is —CH₃ or —CH₂CH₃ or optionally wherein each R¹², independently selected, is —CH₃ or phenyl or two of R¹³ in each —OSi(R¹³)₃, independently selected, are —CH₃ or —CH₂CH₃ and the remaining R¹³ is —CH₃, —CH₂CH₃, t-butyl or phenyl.

35. The process of embodiment 34 wherein R¹² and R¹³ are —CH₃.

36. The process of embodiment 34 wherein R¹² and R¹³ are —CH₃ or two of R¹³ are —CH₃ or —CH₂CH₃ and the remaining R¹³ is —CH₂CH₃, t-butyl or phenyl.

37. The process of embodiment 34 wherein the 3α-O-linked-5α-androstane steroid prepared has the structure

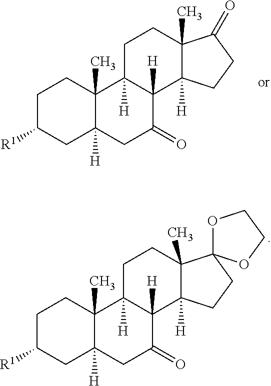

38. The process of embodiment 34 wherein the 3α-O-linked-5α-androstane steroid prepared has the structure

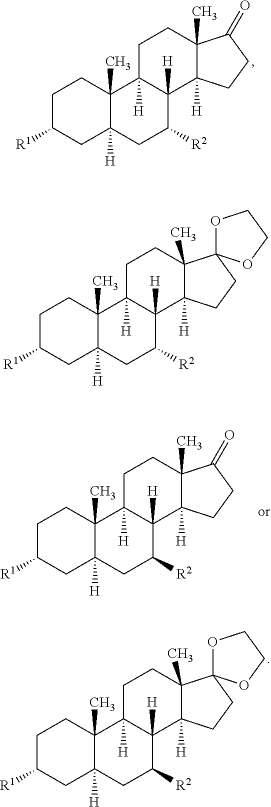

39. The process of embodiment 34 wherein the 3α-O-linked-5α-androstane steroid prepared has the structure

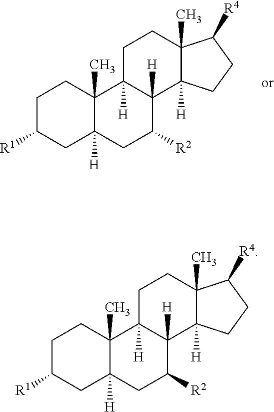

40. The process of embodiment 34 wherein the 3α-O-linked-5α-androstane steroid prepared has the structure

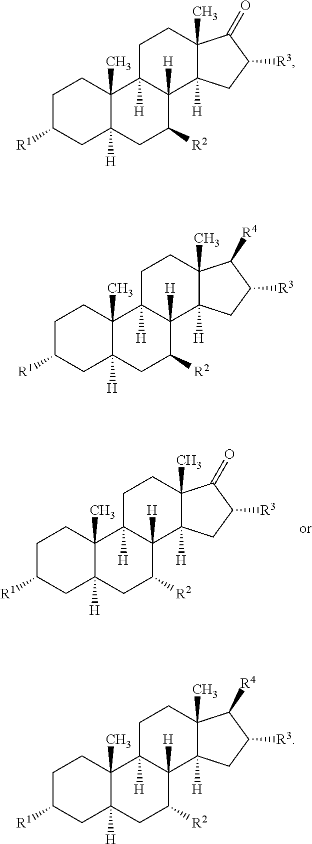

41. The process of embodiment 34 wherein the 3α-O-linked-5α-androstane steroid prepared is 5α-androstan-7,17-dione-3α-ol, 3α-acetoxy-5α-androstan-7,17-dione, 17,17-ethylenedioxy-5α-androstan-7-one-3α-ol, 17,17-ethylenedioxy-3α-acetoxy-5α-androstan-7-one, 5α-androstan-17-one-3α,7α-diol, 17,17-ethylenedioxy-5α-androstane-3α,7α-diol, 5α-androstan-17-one-3α,713-diol, 17,17-ethylenedioxy-5α-androstane-3α,713-diol.

42. The process of embodiment 34 wherein the 3α-O-linked-5α-androstane steroid prepared is 5α-androstane-3α,7α,17β-triol, 5α-androstane-3α,7β,17β-triol, 5α-androstane-3α,7α,16α,17β-tetrol, 5α-androstane-3α,7β,16α,17β-tetrol, 16α-fluoro-5α-androstane-3α,7β,17β-triol, 16α-methoxy-5α-androstane-3α,7β,17β-triol, 16α-methyl-5α-androstane-3α,7β,17β-triol or 16α-propyl-5α-androstane-3α,7β,17β-triol.

43. A process to prepare a 3α-O-linked androst-5-ene steroid or a 3α-O-linked 5α-androstane steroid having disubstitution at position C-17, wherein the 3α-O-linked androst-5-ene steroid or the 3α-O-linked 5α-androstane steroid prepared, optionally after protecting group removal, has the structure

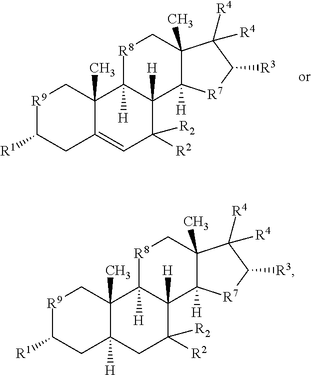

comprising the steps of (1) contacting a suitably protected 3α-O-linked-androst-5-ene, obtained or prepared from the 3α-O-linked-androst-5-ene product of claim 20 or 21, having a ═O moiety (ketone) at position C-17, or a suitably protected 3α-O-linked-5α-androstane, obtained or prepared from the 3α-O-linked-5α-androstane product of claim 29, having a ═O moiety (ketone) at position C-17, with a suitably protected optionally substituted alkyl, optionally substituted alkenyl or optionally substituted alkynyl organometallic anion, whereby the organometallic anion adds to the ═O moiety;

wherein R¹ is a suitable monovalent O-linked moiety; one R² is a suitable monovalent O-linked moiety and the other R² is —H or a suitable O-linked moiety or R² together are —OC(R¹⁶)₂C(R¹⁶)₂O— or —OC(R¹⁶)₂C(R¹⁶)₂C(R¹⁶)₂O— (ketal), wherein R¹⁶ independently are —H or C₁₋₄ alkyl or two of R¹⁶ and the carbon(s) to which they are attached form a cycloalkyl, optionally a C₃, C₅ or C₆ cycloalkyl, and the remaining R¹⁶ are —H; R³ is —H, a suitable monovalent O-linked moiety, a suitable halogen or a suitable monovalent C-linked moiety; one R⁴ is a monovalent O-linked moiety and the other R⁴ is the suitably protected optionally substituted alkyl, optionally substituted alkenyl or optionally substituted alkynyl derived from the organometallic anion; R⁷, R⁸ and R⁹ independently are —C(R¹⁰)₂—, wherein R¹⁰ independently are —H or a suitable monovalent O-linked moiety, optionally wherein (i) R⁷, R⁸ and R⁹ are —CH₂—, (ii) R⁹ and R⁷ are —CH₂— and R⁸ is —C(R¹⁰)₂— wherein one R¹⁰ is α-OH—, β-OH, α-ester, or β-ester and the other R¹⁰ is —H or (iii) R⁷ and R⁸ are —CH₂— and R⁹ is —C(R¹⁰)₂— wherein one R¹⁰ is α-OH—, β-OH, α-ester, or β-ester and the other R¹⁰ is —H; whereby a 3α-O-linked 5α-androstane product or a 3α-O-linked androst-5-ene steroid product having disubstitution at position C-17 is prepared, wherein the monovalent O-linked moiety of R⁴ is —OH; and

(2) optionally contacting the initial oxyanion addition product resulting from step 1 with an electrophile having the structure R¹¹-LG, R¹²—C(O)-LG, (R¹³)₃Si-LG or (R¹⁴)₂N—C(O)-LG wherein LG is a leaving group and R¹¹, R¹², R¹³ and R¹⁴ are a suitable monovalent C-linked moiety, independently selected; whereby a 3α-O-linked 5α-androstane product or a 3α-O-linked androst-5-ene steroid product having disubstitution at position C-17 is prepared,

wherein the monovalent O-linked moiety of R⁴ is an ester, an ether, a silyl ether or a carbamate derived from the electrophile of step 2 and the other R⁴ is the optionally substituted alkyl, optionally substituted alkenyl or optionally substituted alkynyl derived from the organometallic anion of step 1.

44. The process of embodiment 43 wherein the organometallic anion has the structure of M-C≡C—Si(R¹³)₃ wherein R¹³ independently are C₁₋₆ alkyl or aryl and M is a Group I, Group II or transition metal.

45. The process of embodiment 44 wherein M is Na, Li, Mg or Zn, optionally wherein R¹³ are —CH₃.

46. The process of embodiment 45 wherein the 3α-O-linked androst-5-ene steroid prepared or the 3α-O-linked 5α-androstane steroid prepared has the structure

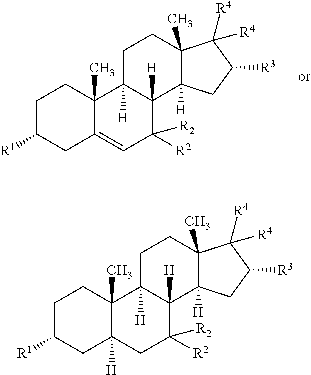

wherein R¹ is —OH, —OR^{PR}, —OR¹¹, —OC(O)—R¹² or —OSi(R¹³)₃; one of R² is —OH, —OR^{PR}, —OR¹¹, —OC(O)—R¹² or —OSi(R¹³)₃ and the other R² is —H or both R² together are ═O; R³ is —H, —OH, —OR^{PR}, —OR¹¹, —OC(O)—R¹², fluoro or optionally substituted alkyl; one R⁴ is —OH, —OR¹¹, —OC(O)—R¹², —OSi(R¹³)₃ and the other R⁴ is an optionally substituted alkynyl wherein the optionally substituted alkynyl has the structure —C≡R; wherein R is CR^{A} and wherein R^{A} is H, optionally substituted alkyl or —Si(R¹³)₃;

wherein R¹¹, R¹² and R¹³ independently are optionally substituted C₁₋₆ alkyl or optionally substituted aryl; and optionally wherein each R¹¹, independently selected, is —CH₃ or —CH₂CH₃, each R¹², independently selected, is —CH₃ or phenyl and two of R¹³ in each —OSi(R₁₃)₃ᵢ independently selected, are —CH₃ or —CH₂CH₃ and the remaining R¹³ are —CH₃, —CH₂CH₃, t-butyl or phenyl.

47. The process of embodiment 45 wherein the 3α-O-linked androst-5-ene steroid prepared has the structure

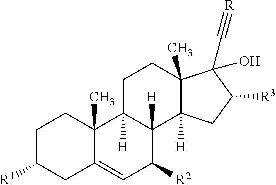

wherein R¹ and R² independently are —OH or —OSi(R¹³)₃; and R³ is —H, —OH or —OSi(R¹³)₃ and R in —C≡R is CR^{A} wherein R^{A} is —H, optionally substituted C₁₋₆ alkyl or —Si(R¹³)₃; wherein R¹³ independently are C₁₋₆ alkyl or aryl; and optionally wherein two of R¹³ in one or more of —OSi(R¹³)₃ or in —Si(R¹³)₃ are —CH₃ or —CH₂CH₃ and the remaining R¹³ are —CH₃, —CH₂CH₃, t-butyl or phenyl, independently selected.

48. The process of embodiment 47 wherein R¹ and R² independently are —OH or —OSi(R¹³)₃ wherein R¹³ are —CH₃; R³ is —H and R^{A} is —Si(CH₃)₃.

49. The process of embodiment 43 wherein the 3α-O-linked androst-5-ene steroid prepared, optionally after deprotection is, 17α-ethynyl-androst-5-ene-3α,7β,17β-triol, 17α-ethynyl-androst-5-ene-3α,7α,17β-triol, 17α-ethynyl-androst-5-ene-3α,7β,16α,17β-tetrol, 17α-ethynyl-androst-5-ene-3α,7α,16α,17β-tetrol, 17α-ethenyl-androst-5-ene-3α,7β,17β-triol, 17α-methyl-androst-5-ene-3α,7β,16α,17β-tetrol, 17α-ethynyl-16α-fluoro-androst-5-ene-3α,7β,17β-triol or 17α-ethynyl-16α-methoxy-androst-5-ene-3α,7β,17β-triol.

50. A compound having the structure

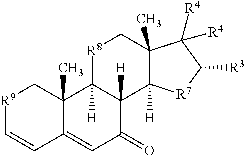

wherein R³ is —H, halogen, a monovalent O-linked moiety or a monovalent C-linked moiety; one R⁴ is a monovalent O-linked moiety and the other R⁴ is —H, a monovalent O-linked moiety or a monovalent C-linked moiety or both R⁴ together are ═O, —O—C(R¹⁶)₂—C(R¹⁶)₂—O— or —O—C(R¹⁶)₂—C(R¹⁶)₂—C(R¹⁶)₂—O—, wherein R¹⁶ independently are —H or C₁₋₄ alkyl or two of R¹⁶ and the carbon(s) to which they are attached comprise a cycloalkyl moiety, optionally C₃, C₅ or C₆ cycloalkyl, and the other R¹⁶ are —H; R⁷ and R⁸ independently are —C(R¹⁰)₂— wherein R¹⁰ independently are —H, a monovalent O-linked, a monovalent C-linked moiety or together are a divalent O-linked moiety; R⁹ is —C(R¹⁰)₂—, wherein R¹⁰ independently are —H, a monovalent O-linked or a monovalent C-linked moiety; provided that R³ is halogen, a monovalent O-linked moiety or a monovalent C-linked moiety when R⁹ is —CH₂—.

51. The compound of embodiment 50 wherein R³ is —H, halogen, optionally bromo, chloro or fluoro, or a monovalent O-linked moiety or a monovalent C-linked moiety, wherein the C-linked moiety is optionally substituted alkyl; one R⁴ is a monovalent O-linked moiety and the other R⁴ is —H, a monovalent C-linked moiety, wherein the monovalent C-linked moiety is optionally substituted alkyl, optionally substituted alkenyl or optionally substituted alkynyl, or a monovalent O-linked moiety or both R⁴ together are ═O or —O—C(R¹⁶)₂—C(R¹⁶)₂—O—; R⁷ and R⁸ are —CH₂—; R⁹ is —C(R¹⁰)₂— wherein one R¹⁰ is —H and the other R¹⁰ is —H or a monovalent O-linked moiety, optionally wherein R⁹ is —CH₂—, —CH(α-OH)— or —CH(β-OH)—;

wherein the monovalent O-linked moieties, independently selected, are —OH, an ester, an ether or a silyl ether.

52. The compound of embodiment 50 wherein the compound is 17,17-ethylenedioxy-16α-fluoro-androst-3,5-dien-7-one, 17,17-ethylenedioxy-androst-3,5-dien-7-one-2α-ol, androst-3,5-dien-7,17-dione-16α-ol, 2α-acetoxy-androst-3,5-dien-7,17-dione, androst-3,5-dien-7,17-dione-2α-ol, 16α-fluoro-androst-3,5-dien-7,17-dione, 16α-methoxy-epoxy-androst-3,5-dien-7,17-dione, 16α-methyl-androst-3,5-dien-7,17-dione or 16α-propyl-androst-3,5-dien-7,17-dione.

53. A compound having the structure

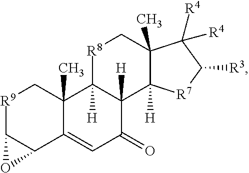

wherein R³ is —H, halogen, a monovalent O-linked moiety or a monovalent C-linked moiety; one R⁴ is a monovalent O-linked moiety and the other R⁴ is —H, a monovalent O-linked moiety or a monovalent C-linked moiety or both R⁴ together are ═O, —X—C(R¹⁶)₂—C(R¹⁶)₂—Y— or —X—C(R¹⁶)₂—C(R¹⁶)₂—C(R¹⁶)₂—Y—, wherein R¹⁶ independently are —H or C₁₋₄ alkyl or two of R¹⁶ and the carbon(s) to which they are attached comprise a cycloalkyl moiety, optionally C₃, C₅ or C₆ cycloalkyl, and the other R¹⁸ are —H; and X and Y independently are O or S; R⁷ and R⁸ independently are —C(R¹⁰)₂— wherein R¹⁰ independently are —H, a monovalent O-linked, a monovalent C-linked moiety or together are a divalent O-linked moiety; and R⁹ is —C(R¹⁰)₂—, wherein R¹⁰ independently are —H, a monovalent O-linked moiety, optionally —OH, a C₂₋₄ ester such as acetate or propionate or a C₁₋₄ ether such as methoxy or ethoxy, or a monovalent C-linked moiety, optionally C₁₋₄ optionally substituted alkyl such as methyl, ethyl, 2-hydroxyethyl, n-propyl or 3-hydroxy-n-propyl, provided that R³ is halogen, a monovalent O-linked moiety or a monovalent C-linked moiety when R⁷, R⁸ and R⁹ are —CH₂— and both R⁴ together are ═O.

54. The compound of embodiment 53 wherein R³ is —H, halogen, optionally bromo, chloro or fluoro, or a monovalent O-linked moiety or a monovalent C-linked moiety, wherein the C-linked moiety is optionally substituted alkyl; one R⁴ is a monovalent O-linked moiety and the other R⁴ is —H, a monovalent C-linked moiety, wherein the monovalent C-linked moiety is optionally substituted alkyl, optionally substituted alkenyl or optionally substituted alkynyl, or a monovalent O-linked moiety or both R⁴ together are ═O or —O—C(R¹⁶)₂—C(R¹⁶)₂—O—, R⁷ and R⁸ are —CH₂—; R⁹ is —C(R¹⁰)₂— wherein one R¹⁰ is —H and the other R¹⁰ is —H or a monovalent O-linked moiety, optionally wherein R⁹ is —CH₂—, —CH(α-OH)— or —CH(β-OH)—;

wherein the monovalent O-linked moieties, independently selected, are —OH, an ester, an ether or a silyl ether, optionally a C₂₋₄ ester such as acetate or propionate or a C₁₋₄ ether such as methyl ether or ethyl ether.

55. The compound of embodiment 54 wherein the compound has the structure

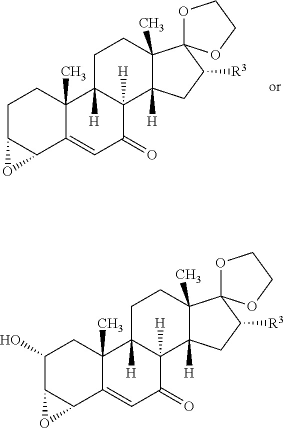

wherein R³ is —H, fluoro, C₁₋₄ alkyl, optionally methyl, ethyl or n-propyl, C₁₋₄ ether, optionally methoxy or ethoxy or C₁₋₄ ester, optionally acetate, or a silyl ether, optionally trimethylsilyloxy or t-butyldimethylsilyloxy.

56. The compound of embodiment 53 wherein the compound is prepared by a process comprising the step of contacting a suitably protected androst-3,5-diene of claim 45 with an epoxidizing agent wherein the epoxidizing agent predominately reacts with the Δ³ functional group in comparison to the Δ⁵ functional group, whereby a 3α,4α-epoxy-androst-5-en-7-one steroid product is obtained.

57. The compound of embodiment 53 wherein the compound is 17,17-ethylenedioxy-3α,4α-epoxy-androst-5-en-7-one, 17,17-ethylenedioxy-3α,4α-epoxy-androst-5-en-7-one-2α-ol, 3α,4α-epoxy-androst-5-en-7,17-dione-16α-ol, 2α-acetoxy-3α,4α-epoxy-androst-5-en-7,17-dione, 3α,4α-epoxy-androst-5-en-7,17-dione-2α-ol, 16α-fluoro-3α,4α-epoxy-androst-5-en-7,17-dione, 16α-methoxy-3α,4α-epoxy-androst-5-en-7,17-dione, 16α-methyl-3α,4α-epoxy-androst-5-en-7,17-dione or 16α-propyl-3α,4α-epoxy-androst-5-en-7,17-dione.

58. A process to prepare a 3α-O-linked-androst-5-ene steroid comprising, (1) contacting a suitably protected 3β-hydroxy steroid with an azo-di-carboxylate ester, a tri-substituted phosphine and an organic acid having the structure of R¹²C(O)OH wherein R¹² is C₁₋₆ alkyl, C₃₋₆ cycloalkyl or optionally substituted aryl, wherein the suitably protected 3β-hydroxy steroid has the structure

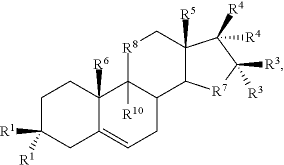

wherein R¹ in the 6-configuration is —OH and R¹ in the α-configuration is —H or a suitable optionally substituted alkyl, optionally a C₁₋₄ optionally substituted alkyl such as methyl, ethyl or n-propyl; R³ independently or together are —H, halogen, a suitable C-linked moiety, a suitable monovalent O-linked moiety, ═O (ketone), —O—C(R¹⁶)₂—C(R¹⁶)₂—O— or —O—C(R¹⁶)₂—C(R¹⁶)₂—C(R¹⁶)₂—O— (ketal); R⁴ in the β-configuration is a suitable monovalent O-linked moiety; R⁴ in the α-configuration is —H or a suitable C-linked moiety or R⁴ together are ═O (ketone), —O—C(R¹⁶)₂—C(R¹⁶)₂—O— or —O—C(R¹⁶)₂—C(R¹⁶)₂—C(R¹⁶)₂—O— (ketal); R⁵ and R⁶ independently are —H or a suitable optionally substituted alkyl, optionally a C₁₋₄ optionally substituted alkyl such as —CH₃, —C₂H₅ or —C₂H₄OH; R⁷ and R⁸ independently are —C(R¹⁰)₂—; wherein R¹⁰ independently or together are —H, a suitable halogen, a suitable monovalent C-linked moiety or a suitable monovalent O-linked moiety or both R¹⁰ together are ═O, —O—C(R¹⁶)₂—C(R¹⁶)₂—O— or —O—C(R¹⁶)₂—C(R¹⁶)₂—C(R¹⁶)₂—O— (ketal); R¹⁰ at position C-9 is —H or halogen, optionally —F; R^{PR} independently are —H or protecting group;

wherein the C-linked moieties are independently a suitable optionally substituted alkyl group, optionally substituted alkenyl group or optionally substituted alkynyl group; and wherein the monovalent O-linked moieties independently are —OR^{PR} an ester or an ether;

wherein R¹⁶ independently are —H or C₁₋₄ alkyl or two of R¹⁶ and the carbon(s) to which they are attached form a cycloalkyl, optionally C₃, C₅ or C₆ cycloalkyl;

wherein the molar ratio of the azo-di-carboxylate ester to the 3β-hydroxy steroid is less than 1.5:1 and greater than 1.0:1, whereby a 3α-androst-5-ene product having a 3α-O-linked ester substantially free of 3α,5α-cycloandrostane side-products is obtained; and

(2) optionally contacting the 3α-O-linked ester androst-5-ene from step 1 with a basic solution to convert the 3α-O-ester to 3α-OH.

59. The process of embodiment 58 wherein the molar ratio of the azo-di-carboxylate ester to the 3β-hydroxy steroid is about 1.3:1.

60. The process of embodiment 58 wherein the azo-di-carboxylate ester, tri-substituted phosphine and organic acid are in substantially equimolar amounts.

61. The process of embodiment 58, 59 or 60 wherein R¹⁹ of the organic acid is an optionally substituted phenyl wherein the 3α-O-linked ester androst-5-ene obtained or prepared from the product of step 1 is capable of hydrolysis in an aqueous solution at ambient temperature to provide a 3α-hydroxy-androst-5-ene steroid.

62. The process of claim 61 wherein the an azo-di-carboxylate ester is added to a mixture of the tri-substituted phosphine, organic acid and p-hydroxy steroid at between about 0 to 25° C.

63. The process of embodiment 62 wherein the azo-di-carboxylate ester is added to a mixture of the tri-substituted phosphine at a temperature of between about 0-10° C. whereupon the mixture is warmed to between about 10-25° C.

64. The process of claim 58 embodiment R¹⁹ is p-NO₂-phenyl and the azo-di-carboxylate ester has the structure R¹⁹OC(O)N═NC(O)OR¹⁹ wherein R¹⁹ is —CH₂CH₃ (DEAD) or —CH(CH₃)₂ (DIAD).

65. The process of embodiment 62 wherein 3α-O-linked-androst-5-ene steroid prepared, optionally after protecting group removal, has the structure

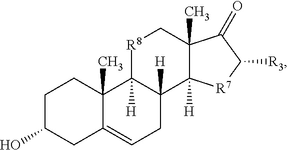

wherein R³ is —H, halogen, a monovalent O-linked moiety or a monovalent C-linked moiety; R⁷ and R⁸ independently are —C(R¹⁰)₂, wherein R¹⁰ independently are —H a monovalent O-linked moiety or a monovalent C-linked moiety.

66. The process of embodiment 65 wherein 3α-O-linked-androst-5-ene steroid prepared is androst-5-en-17-one-3α-ol (3α-DHEA), androst-5-en-17-one-3α,116-diol, androst-5-en-17-one-3α,15α-diol, androst-5-en-17-one-3α,15α,16α-triol, androst-5-en-17-one-3α,116,16α-triol, 16α-fluoro-androst-5-en-17-one-3α-ol.

67. A process to prepare a 3α-O-linked-5α-androstane steroid comprising the steps of contacting a suitably protected 3α-O-linked androst-5-ene prepared or obtained from the 3α-O-linked-androst-5-ene product of claim 58 with a hydrogen donor to reduce the Δ⁵ functional group, whereby a 3α-O-linked-5α-androstane product is obtained.

68. A process to prepare a 3α-O-linked androst-5-ene steroid or a 3α-O-linked 5α-androstane steroid having disubstitution at position C-17 comprising the steps of (1) contacting a suitably protected 3α-O-linked-androst-5-ene obtained or prepared from the 3α-O-linked-androst-5-ene product, having a ═O moiety (ketone) at position C-17 of claim 58 or a suitably protected 3α-O-linked-5α-androstane obtained or prepared from the 3α-O-linked-5α-androstane steroid product of embodiment 67, having a ═O moiety (ketone) at position C-17, with a suitably protected optionally substituted alkyl, an optionally substituted alkenyl or an optionally substituted alkynyl organometallic anion, whereby the organometallic anion adds to the ═O moiety to provide a 3α-O-linked 5α-androstane product or a 3α-O-linked 5α-androstane product having disubstitution at position C-17; and

(2) optionally contacting the initial oxyanion addition product resulting from step 1 with an electrophile having the structure R¹¹-LG, R¹²—C(O)-LG, LG, (R¹³)₃Si-LG or (R¹⁴)₂N—C(O)-LG wherein LG is a leaving group and R¹¹, R¹², R¹³ and R¹⁴ are a suitable monovalent C-linked moiety, whereby a 3α-O-linked 5α-androstane product or a 3α-O-linked androst-5-ene steroid product having disubstitution at position C-17 is prepared, wherein one C-17 substituent is a monovalent O-linked moiety, wherein the monovalent O-linked moiety is —OH or an ester, an ether, silyl ether or a carbamate derived from the electrophile of step 2 and the other C-17 substituent is the optionally substituted alkyl, optionally substituted alkenyl or optionally substituted alkynyl of step derived from the organometallic anion of step 1.

69. The process of embodiment 68 wherein the organometallic anion has the structure of M-C≡C—Si(R¹³)₃ wherein R¹³ independently are C₁₋₆ alkyl or aryl and M is a Group I, Group II or transition metal.

70. The process of embodiment 69 wherein M is Na, Li, Mg or Zn.

71. The process of embodiment 68 wherein the 3α-O-linked androst-5-ene steroid or the 3α-O-linked 5α-androstane steroid prepared, optionally after protecting group removal, is 17α-ethynyl-androst-5-ene-3α,17β-diol, 17α-ethynyl-5α-androstane-3α,17β-diol, 17α-ethenyl-5α-androstane-3α,17β-diol, 17α-ethyl-5α-androstane-3α,17β-diol, 17α-methyl-androst-5-ene-3α,17β-diol, 17α-ethynyl-16α-fluoro-5α-androstane-3α,17β-diol, 17α-ethynyl-16α-methoxy-5α-androstane-3α,17β-diol, 17α-ethynyl-16α-fluoro-androst-5-ene-3α,17β-diol, 17α-ethynyl-androst-5-ene-3α,16α,17β-triol or 17α-ethynyl-5α-androstane-3α,16α,17β-triol.

1A. A compound having the structure

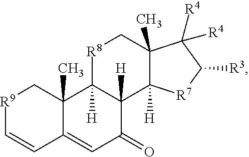

wherein R³ is —H, halogen, a monovalent O-linked moiety or a monovalent C-linked moiety; one R⁴ is a monovalent O-linked moiety and the other R⁴ is —H, a monovalent O-linked moiety or a monovalent C-linked moiety or both R⁴ together are a divalkent O-linked moiety such as ═O, —O—C(R¹⁶)₂—C(R¹⁶)₂—O— or —O—C(R¹⁶)₂—C(R¹⁶)₂—C(R¹⁶)₂—O—, wherein R¹⁶ independently are —H or C₁₋₄ alkyl or two of R¹⁶ and the carbon(s) to which they are attached comprise an optionally substituted C₃, C₅ or C₆ cycloalkyl or one of R¹⁶ and the carbon to which it is attached defines a C₃, C₅ or C₆ or spiroalkyl moiety, and the other R¹⁶ are —H; R⁷ and R⁸ independently are —C(R¹⁰)₂— wherein R¹⁰ independently are —H, a monovalent O-linked, a monovalent C-linked moiety or together are a divalent O-linked moiety; R⁹ is —C(R¹⁰)₂—, wherein R¹⁰ independently are —H, a monovalent O-linked, a monovalent C-linked moiety or a halogen, provided that R³ is halogen, a monovalent O-linked moiety or a monovalent C-linked moiety when R⁹ is —CH₂— or R⁷, R⁸ or R⁹ are —CH₂.

In this embodiment preferred O-linked moieties are —OH, —OR^{PR}, wherein R^{PR} is a hydroxy protecting group, substituted or unsubstituted C₁₋₆ alkyl ester, substituted or unsubstituted C6 aryl esters, substituted or unsubstituted alkyl C₁₋₆ ethers, substituted or unsubstituted C₆ aryl ethers or substituted or unsubstituted silyl ethers.

Preferred C₁₋₆ alkyl esters (i.e. acyloxy substituents) are formate (a C₁ alkyl ester), acetate (a C₂ alkyl ester), propionate (a C₃ alkyl ester) and phenylacetate (a phenyl substituted C₂ alkyl ester). Preferred C₆ aryl esters (i.e., arylcarbonyloxy substituents) are benzoyl, p-nitrophenyl, 2,4-dinitrophenyl, p-fluorophenyl, p-chlorophenyl, p-bromophenyl and p-methylphenyl (p-toulyl) ester. Particularly preferred esters are acetate, propionate, benzoate, phenylacetate and p-nitrophenyl ester with acetate especially preferred.

Preferred C₁₋₆ alkyl ethers are methyl, ethyl, methoxymethyl, ethoxymethyl, tetrahydrofuranyl and tetrahydropyranyl ether with methoxy ether particularly preferred. Preferred C6 aryl ethers are pheny, p-methoxyphenyl, o-methylohenyl (o-toluoyl), o-methoxyphenyl and 2,4-dimethoxyphenyl ethers.

Preferred silyl ethers are trimethylsilyl, triethylsilyl, tert-butyldiphenylsilyl, tert-butyldimethylsilyl, triisopropylsilyl (TIPS) and [2-(trimethylsilyl)ethoxy]methylsilyl ether with trimethylsilyl and tert-butyldimethylsilyl particularly preferred and trimethylsilyl especially preferred.

Preferred divalent O-linked moities have the structure —X—C(R¹⁶)₂—C(R¹⁶)₂—Y— or —X—C(R¹⁶)₂—C(R¹⁶)₂—C(R¹⁶)₂—Y—, where both X and Y are O or S. In particularly preferred embodiments X and Y are both —O. In other preferred embodiments the di-valent O-linked moiety has the structure —X—C(R¹⁶)₂—C(R¹⁶)₂—Y— where R¹⁶ are all —H or —CH₃. Particularly preferred are those di-valent O-linked moities where both X and Y are —H and R¹⁶ are all —H with —OCH₂CH₂O— especially preferred.

Preferred monovalent C-linked moities are C₁₋₆ alkyl, C₂₋₆ alkenyl and C₂₋₆ alkynyl groups. Particularly preferred are methyl, ethyl, propyl, isopropyl, —CH₂OH, CH₂OR^{PR}, vinyl, E-2-chloro-ethen-1-yl, E-2-bromo-ethen-1-yl, E-2-iodo-ethen-1-yl, ethynyl, propynyl, phenylethynyl and chloroethynyl with ethynyl (—C≡CH) and methyl (—CH₃) especially preferred.

Preferred halogens are fluoro, chloro and bromo with fluoro and chloro particularly preferred.

Preferred moities for R⁹ is —C(R¹⁰)₂— are those moieties wherein both R¹⁰ are —H or R¹⁰ in the β-configuration is halogen, C₁₋₆ alkyl, C₁₋₆ ester, C₁₋₆ ether or —OR^{PR}, where R^{PR} is a hydroxyl protecting group. Other preferred R⁹ moities as those wherein R¹⁰ in the β-configuration is C₁₋₆ alkyl, chloro or fluoro, and R¹⁰ in the α-configuration is —H or —OH or R¹⁰ in the α-configuration is —OH and R¹⁰ in the β-configuration is —H.

Preferred moieties for R⁷ is —C(R¹⁰)₂— and R⁸ is —C(R¹⁰)₂— are those independently selected moities where both R¹⁰ are —H, one R¹⁰ in the α- or β-configuration is a monovalent O-linked moiety or halogen and the other R¹⁰ is —H or both R¹⁰ comprise a divalent O-linked moiety. Preferred halogen and monovalent and divalent O-linked moieties are —Br, —Cl, —OR^{PR}, —OC(O)CH₃ (acetate), —OMe, —OTHP, —OSi(CH₃)₃ and —OCH₂CH₂O—. In other preferred embodiments R⁷ and R⁸ are —CH₂—.

2A. The compound of embodiment 1A wherein R³ is —H, halogen, optionally bromo, chloro or fluoro, or a monovalent O-linked moiety or a monovalent C-linked moiety, wherein the C-linked moiety is optionally substituted alkyl; one R⁴ is a monovalent O-linked moiety and the other R⁴ is —H, a monovalent C-linked moiety, wherein the monovalent C-linked moiety is optionally substituted alkyl, optionally substituted alkenyl or optionally substituted alkynyl, or a monovalent O-linked moiety or both R⁴ together are ═O or —O—C(R¹⁶)₂—C(R¹⁶)₂—O—; R⁷ and R⁸ are —CH₂—; R⁹ is —C(R¹⁰)₂—, wherein one R¹⁰ is —H and the other R¹⁰ is —H or a monovalent O-linked moiety or R⁹ is —CH₂—, —CH(α-OH)— or —CH(β-OR^{PR})—; wherein the monovalent O-linked moieties, independently selected, are —OH, an ester, an ether or a silyl ether.

3A. The compound of embodiment 1A wherein the compound is 17,17-ethylenedioxy-16α-fluoro-androst-3,5-dien-7-one, 17,17-ethylenedioxy-androst-3,5-dien-7-one-2α-ol, androst-3,5-dien-7-17-dione-16α-ol, 2α-acetoxy-androst-3,5-dien-7,17-dione, androst-3,5-dien-7-17-dione-2α-ol, 16α-fluoro-androst-3,5-dien-7,17-dione, 16α-methoxy-epoxy-androst-3,5-dien-7,17-dione, 16α-methyl-androst-3,5-dien-17-one, 16α-propyl-androst-3,5-dien-17-one or 16α-(prop-2-yl)-androst-3,5-dien-17-one. In other embodiments the androst-3,5-dien-7-one compound is any of one these enumerated compounds represented by the formula of embodiment 1A wherein one or more additional mono-O-linked substituents such as hydroxy or acetoxy are present independently in R⁷, R⁸ and R⁹.

Preferred are those compounds additionally having one of R⁷, R⁸, R⁹ as —C(R¹⁰)₂— wherein R¹⁰ in the α- or β-configuration is —OH or acetate and the other R¹⁰ is —H.

4A. A compound having the structure

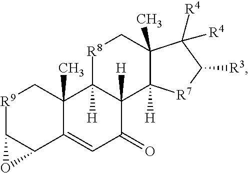

wherein R³ is —H, halogen, a monovalent O-linked moiety or a monovalent C-linked moiety; one R⁴ is a monovalent O-linked moiety and the other R⁴ is —H, a monovalent O-linked moiety or a monovalent C-linked moiety or both R⁴ together are ═O or —X—C(R¹⁶)₂—C(R¹⁶)₂—Y—, wherein R¹⁶ independently are —H or C₁₋₄ alkyl or two of R¹⁶ and the carbon(s) to which they are attached comprise an optionally substituted C₃, C₅ or C₆ cycloalkyl or one of R¹⁶ and the carbon to which it is attached defines a C₃, C₅ or C₆ or spiroalkyl moiety, and the other R¹⁶ are —H; and X and Y independently are O or S; R⁷ and R⁸ independently are —C(R¹⁰)₂— wherein R¹⁰ independently are —H, a monovalent O-linked, a monovalent C-linked moiety or together are a divalent O-linked moiety; R⁹ is —C(R¹⁰)₂—, wherein R¹⁰ independently are —H, a monovalent O-linked moiety, a monovalent C-linked moiety, provided that R³ is halogen, a monovalent O-linked moiety or a monovalent C-linked moiety when R⁷, R⁸ and R⁹ are —CH₂— and both R⁴ together are ═O.

Preferred halogen and monovalent and divalent O-linked moieties are —Br, —Cl, —OH—OR^{PR}, —OC(O)CH₃, —OMe, —OTHP, —OSi(CH₃)₃ and —OCH₂CH₂O—. In some embodiments R⁷, R⁸ and R⁹ are those independently selected moities where both R¹⁰ are —H, one R¹⁰ in the α- or β-configuration is a monovalent O-linked moiety or halogen and the other R¹⁰ is —H or both R¹⁰ comprise a divalent O-linked moiety. Preferred R¹⁰ substituents in R⁷, R⁸ and R⁹ are those described in this embodiment and in embodiment 1A for R⁷ and R⁸. In some preferred embodiments R⁹ is —CH₂—, —C(α-H, β-OR^{PR})— or —C(α-OH, (βH)—. In other preferred embodiments R⁷ and R⁸ are —CH₂—.

In some process embodiments an —OH substituent in a 3α,4α-epoxy-androst-5-ene is derived from an —OR^{PR} moiety in a precursor used to prepare that 3α,4α-epoxy-androst-5-ene. This is particularly advantageous in processes described herein when the product obtained is to have R⁹ is —C(R¹⁰)₂— where R¹⁰ in the β-configuration is —OH.

5A. The compound of embodiment 4A wherein R³ is —H, halogen, optionally bromo, chloro or fluoro, or a monovalent O-linked moiety or a monovalent C-linked moiety, wherein the C-linked moiety is optionally substituted alkyl; one R⁴ is a monovalent O-linked moiety and the other R⁴ is —H, a monovalent C-linked moiety, wherein the monovalent C-linked moiety is optionally substituted alkyl, optionally substituted alkenyl or optionally substituted alkynyl, or a monovalent O-linked moiety or both R⁴ together are ═O or —O—C(R¹⁶)₂—C(R¹⁶)₂—O—; R⁷ and R⁸ are —CH₂—; R⁹ is —C(R¹⁰)₂— wherein one R¹⁰ is —H and the other R¹⁰ is —H or a monovalent O-linked moiety or R⁹ is —CH₂—, —CH(α-OH)— or —CH(β-OH)—; wherein the monovalent O-linked moieties, independently selected, are —OH, an ester, an ether or a silyl ether, optionally a C₂₋₄ ester or a C₁₋₄ ether.

6A. The compound of embodiment 4A wherein the compound has the structure

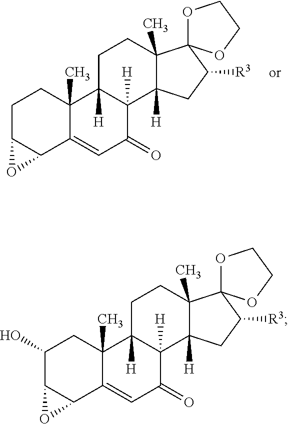

wherein R³ is —H, fluoro, C₁₋₄ alkyl, C₁₋₄ ether, C₁₋₄ ester or a silyl ether.

7A. The compound of embodiment 4A wherein the compound is prepared by a process comprising the step of contacting a suitably protected androst-3,5-diene of claim 1 with an epoxidizing agent wherein the epoxidizing agent selectively reacts with the Δ³ functional group relative to the Δ⁵ functional group, wherein a 3α,4α-epoxy-androst-5-en-7-one steroid product is obtained.

8A. The compound of embodiment 7A wherein the compound is 17,17-ethylenedioxy-3α,4α-epoxy-androst-5-en-7-one, 17,17-ethylenedioxy-3α,4α-epoxy-androst-5-en-7-one-2α-ol, 3α,4α-epoxy-androst-5-en-7,17-dione-16α-ol, 2α-acetoxy-3α,4α-epoxy-androst-5-en-7,17-dione, 3α,4α-epoxy-androst-5-en-7,17-dione-2α-ol, 16α-fluoro-3α,4α-epoxy-androst-5-en-7,17-dione, 16α-methoxy-3α,4α-epoxy-androst-5-en-7,17-dine, 16α-methyl-3α,4α-epoxy-androst-5-en-7,17-dione, 16α-propyl-3α,4α-epoxy-androst-5-en-7,17-one or 16α-(prop-2-yl)-3α,4α-epoxy-androst-5-en-7,17-one.

In other embodiments the 3α,4α-epoxy-androst-5-ene compound is any of one these enumerated compounds represented by the formula of embodiment 4A wherein one or more additional mono-O-linked substituents such as hydroxy or acetoxy are present independently in R⁷, R⁸ and R⁹. Preferred are those compounds additionally having one of R⁷, R⁸, R⁹ as —C(R¹⁰)₂— wherein R¹⁰ in the α- or β-configuration is —OH or acetate and the other R¹⁰ is —H

9A. A process to prepare a 3α-O-linked androst-5-ene steroid comprising the step of (1) contacting a suitably protected 3α,4α-epoxy-androst-5-ene with a first hydrogen donor, wherein the 3α,4α epoxy functional group is selectively reduced relative to the Δ⁵ functional group and wherein reduction of the 3α,4α epoxy functional group occurs preferentially at position C4 with retention of configuration at position position C3,

wherein the suitably protected 3α,4α-epoxy-androst-5-ene has the structure

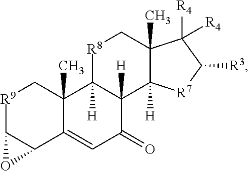

wherein R³ is —H, a suitable halogen, a suitable monovalent O-linked moiety or a suitable monovalent C-linked moiety; and R⁴ independently are an ether or both R⁴ together are —OC(R¹⁶)²C(R¹⁶)₂O— or —OC(R¹⁶)₂C(R¹⁶)₂C(R¹⁶)₂O— (ketal), wherein R¹⁶ independently are —H or C₁₋₄ alkyl or two of R¹⁶ and the carbon(s) to which they are attached form a C₃, C₅ or C₆ cycloalkyl or C₃, C₅ or C₆ spiroalkyl, and the remaining R¹⁶ are —H; and R⁹, R⁷ and R⁸ independently are —C(R¹⁰)₂, wherein R¹⁰ independently are —H or a suitable monovalent O-linked moiety or together form a ketal.

10A. The process of embodiment 9A wherein the first hydrogen donor selectively reduces the 3α,4α epoxy functional group in preference to the C7 ketone functional group, whereby a 3α-O-linked androst-5-ene product having a ═O (ketone) moiety at position C-7 is obtained.

11A. The process of embodiment 9A further comprising the step of (2) contacting the product obtained or prepared from claim 9A with an electrophile, wherein a monovalent O-linked group is obtained at position C3, wherein the monovalent O-linked group so obtained is other than —OH.

12A. The process of embodiment 9A further comprising the step of (3) contacting a suitably protected 3α-O-linked androst-5-en-7-one obtained or prepared from the 3α-O-linked androst-5-ene product of step (1) with a second hydrogen donor, wherein the suitably protected 3α-O-linked androst-5-en-7-one has the structure

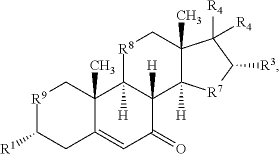

wherein R¹ is a suitable monovalent O-linked moiety; R³ is —H, a suitable C-linked moiety, a suitable halogen or a suitable monovalent O-linked moiety; R⁴ independently are an ether or one R⁴ is a suitable monovalent O-linked moiety and the other R⁴ is —H or both R⁴ together are ═O (ketone) or —OC(R¹⁶)₂C(R¹⁶)₂O— (ketal), wherein R¹⁶ independently are —H or C₁₋₄ alkyl or two of R¹⁶ and the carbon(s) to which they are attached form a C₃, C₅ or C₆ cycloalkyl, and the remaining R¹⁶ are —H; wherein a 3α-O-linked androst-5-ene product having —OH in the α- or β-configuration at position C7 is obtained.

13A. The process of embodiment 12A further comprising the step of (4) contacting the product obtained or prepared from claim 12A with an electrophile having the structure R¹¹-LG, R¹²—C(O)-LG, LG, (R¹³)₃Si-LG or (R¹⁴)₂N—C(O)-LG wherein LG is a leaving group and R¹¹, R¹², R¹³ and R¹⁴ are a suitable monovalent C-linked moiety, independently selected; wherein a 3α-O-linked androst-5-ene product having a monovalent O-linked moiety at position C7 is obtained wherein the monovalent O-linked moiety so obtained is an ether, an ester, a silyl ether or a carbamate. Preferred LG moities include —F, —Cl, —Br, —I, benzenesulfonate, p-toluenesulfonate, triflate and N-hydroxysuccinate.

14A. The process of embodiment 12A wherein the suitably protected 3α-O-linked androst-5-en-7-one contacted with the second hydrogen donor has the structure

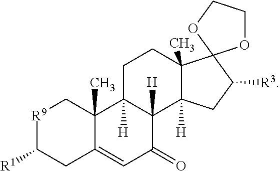

15A. The process of embodiment 9A wherein the first hydrogen donor is provided by Pd(0)/H₂.

16A. The process of embodiment 9A wherein the first hydrogen donor is provided by Pd(0)/H₂, wherein the palladium catalyst is supported on carbon black and is suspended in an alcohol-based solvent in the presence of a carbonate salt to which is applied a hydrogenation temperature of between about ambient or about 40° C. or about 22° C. to about 40° C. and a hydrogenation pressure of between about 15.5 psi to about 50 psi H₂) wherein the 3α,4α-epoxy functionality is selectively reduced relative to the C7 ketone functional group and whereby reduction of the 3α,4α epoxy functional group occurs preferentially at position C4 with retention of configuration at position C3.

17A. The process of embodiment 16A wherein the hydrogenation temperature is ambient or about 22° C., the hydrogenation pressure is about 22 psi H₂, the carbonate salt is potassium carbonate and the alcohol-based solvent is a mixture of ethanol and ethyl acetate in about 5:1 by volume ratio.

18A. The process of embodiment 12A wherein the second hydrogen donor is a suitable hydride reducing agent.

19A. The process of embodiment 9A wherein the suitable monovalent O-linked moieties independently are an ether, —OSi(R¹³)₃, or —OR^{PR}, wherein R^{PR} is —H, a protecting group and R¹³ independently are C₁₋₄ alkyl or aryl, the suitable halogen in R³ is fluoro; and the suitable monovalent C-linked moiety is optionally substituted alkyl, suitably protected.

20A. The process of embodiment 9A wherein the suitably protected 3α,4α-epoxy-androst-5-ene is 17,17-ethylenedioxy-3α,4α-epoxy-androst-5-en-7-one, 17,17-di-methoxy-3α,4α-epoxy-androst-5-en-7-one, 17,17-di-ethoxy-3α,4α-epoxy-androst-5-en-7-one, 17,17-(propylene-1,3-dioxy)-3α,4α-epoxy-androst-5-en-7-one, 17,17-tetramethyl-ethylenedioxy-3α,4α-epoxy-androst-5-en-7-one, 17,17-(cyclohex-1,2-yl)-dioxy-3α,4α-epoxy-androst-5-en-7-one, 17,17-ethylenedioxy-16α-methoxy-3α,4α-epoxy-androst-5-en-7-one, 17,17-ethylenedioxy-16α-fluoro-3α,4α-epoxy-androst-5-en-7-one, 17,17-ethylenedioxy-16α-trimethylsilyloxy-3α,4α-epoxy-androst-5-en-7-one or 17,17-ethylenedioxy-16α-(t-butyl-dimethylsilyl)oxy-3α,4α-epoxy-androst-5-en-7-one.

In other embodiments the 3α,4α-epoxy-androst-5-ene compound is any of one these enumerated compounds represented by the formula of embodiment 9A wherein one or more additional suitable mono-O-linked substituents such as —OR^{PR}, —OTMS, OTBDMS or acetoxy are present independently in R⁷, R⁸ and R⁹. Preferred are those compounds additionally having one of R⁷, R⁸, R⁹ as —C(R¹⁰)₂— wherein R¹⁰ in the α- or β-configuration is —OR^{PR}, —OTMS, —OTBDMS or acetoxy and the other R¹⁰ is —H

21A. The process of any one of embodiment 9A-13A wherein the 3α-O-linked-androst-5-ene steroid prepared, optionally after protecting group removal, has the structure

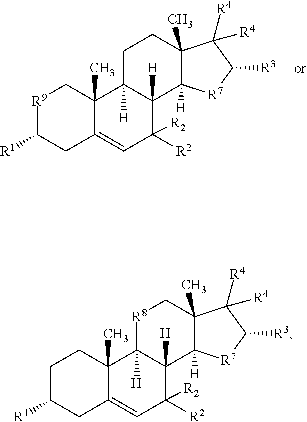

wherein R¹ is —OH, —OR¹¹, —OC(O)—R¹² or —OSi(R¹³)₃; one of R² is —OH, —OR¹¹, —OC(O) —R¹² or —OSi(R¹³)₃ and the other R² is —H or both R² together are ═O; R³ is —H, —OH, —OR¹¹, —OC(O)—R¹²—OSi(R¹³)₃, halogen or C₁₋₄ alkyl; one R⁴ is —H and the other R⁴ is —OH, —OR¹¹, —OC(O)—R¹², —OSi(R¹³)₃ or R⁴ independently or together are —OH, —OR¹¹, —OC(O)—R¹², —OSi(R¹³)₃, ═O or —OC(R¹⁶)₂C(R¹⁶)₂O—; R⁷ and R⁸ independently are —C(R¹⁰)₂— wherein both R¹⁰ are —H or one R¹⁰ is α-OH—, β-OH, α-ester, or β-ester and the other R¹⁰ is —H; R⁹ is —C(R¹⁰)₂—, wherein one R¹⁰ is α-OH, α-OH, α-ester or β-ester and the other R¹⁰ is —H; R¹¹, R¹² and R¹³ independently are optionally substituted C₁₋₆ alkyl or optionally substituted aryl; and R¹⁶ independently are —H or C₁₋₄ alkyl or two of R¹⁶ and the carbon(s) to which they are attached form a cycloalkyl, optionally C₃, C₅ or C₆ cycloalkyl, and the remaining R¹⁶ are —H;

wherein the optionally substituted C₁₋₆ alkyl of each R¹¹, independently selected, is —CH₃ or —CH₂CH₃; wherein each R¹², independently selected, is —CH₃ or phenyl or two of R¹³ in each —OSi(R¹³)₃, independently selected, are —CH₃ or —CH₂CH₃ and the remaining R¹³ is CH₃, —CH₂CH₃, t-butyl or phenyl.

22A. The process of embodiment 21A wherein the 3α-O-linked-androst-5-ene steroid prepared has the structure

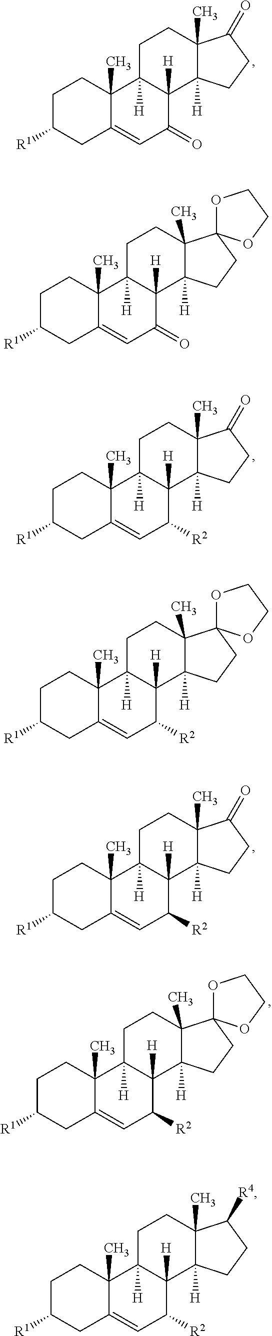

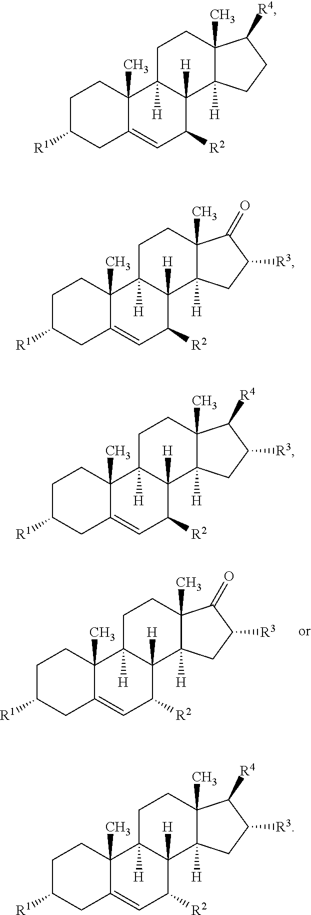

23A. The process of embodiment 21A wherein the 3α-O-linked androst-5-ene steroid prepared, optionally after deprotection, is androst-5-en-7,17-dione-3α-ol, 3α-acetoxy-androst-5-en-7,17-dione, 17,17-ethylenedioxy-androst-5-en-7-one-3α-ol, 17,17-ethylenedioxy-3α-acetoxy androst-5-en-7-one, androst-5-en-17-one-3α,76-diol, 3α-acetoxy-androst-5-en-17-one-76-ol, androst-5-en-17-one-3α,7α-diol, 3α-acetoxy-androst-5-en-17-one-7α-ol, 17,17-ethylenedioxy-androst-5-ene-3α,76-diol, 17,17 ethylenedioxy-3α-acetoxy-androst-5-ene-76-ol, 17,17-ethylenedioxy-androst-5-ene-3α,7α-diol, 17,17-ethylenedioxy-3α-acetoxy-androst-5-ene-7α-ol, androst-5-en-17-one-3α,76,16α-triol, 16α-methoxy-androst-5-en-17-one-3α,76-diol, 16α-fluoro-androst-5-en-17-one-3α,76-diol, androst-5-ene-3α,76,16α,17β-tetrol, 16α-methoxy-androst-5-ene-3α,7β,17β-triol, 16α-fluoro-androst-5-ene-3α,7β,17β-triol, androst-5-en-17-one-3α,7α,16α-triol, 16α-methoxy-androst-5-en-17-one-3α,7α-diol, 16α-fluoro-androst-5-en-17-one-3α,7α-diol, androst-5-ene-3α,7α,16α,17β-tetrol, 16α-methoxy-androst-5-ene-3α,7α,17β-triol or 16α-fluoro-androst-5-ene-3α,7α,17β-triol.

In other embodiments the 3α-Olinked-androst-5-ene compound is any of one these enumerated compounds represented by the formula of embodiment 21A wherein one or more additional suitable mono-O-linked substituents such as —OR^{PR}, —OTMS, OTBDMS or acetoxy are present independently in R⁷, R⁸ and R⁹. Preferred are those compounds additionally having one of R⁷, R⁸, R⁹ as —C(R¹⁰)₂— wherein R¹⁰ in the α- or β-configuration is —OR^{PR}, —OTMS, —OTBDMS or acetoxy and the other R¹⁰ is —H.

24A. The process of embodiment 21A, further comprising the step of (5) contacting a suitably protected 3α-O-linked androst-5-ene prepared or obtained from the 3α-O-linked androst-5-ene product of claim 21A with a third hydrogen donor to reduce the Δ⁵ functional group, wherein a 3α-O-linked-5α-androstane product is obtained.

25A. The process of embodiment 24A wherein the 3α-O-linked-5α-androstane steroid prepared, optionally after protecting group removal, has the structure

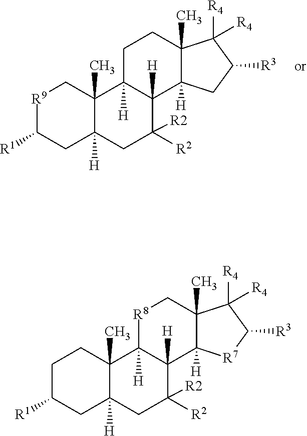

wherein R¹ is —OH, —OR¹¹, —OC(O)—R¹² or —OSi(R¹³)₃; one of R² is —OH, —OR¹¹, —OC(O)—R¹² or —OSi(R¹³)₃ and the other R² is —H or both R² together are ═O; R³ is —H, —OH, —OR¹¹, —OC(O)—R¹²—OSi(R¹³)₃, halogen or C₁₋₄ alkyl; one R⁴ is —H and the other R⁴ is —OH, —OR¹¹, —OC(O)—R¹², —OSi(R¹³)₃ or R⁴ independently or together are —OH, —OR¹¹, —OC(O)—R¹², —OSi(R¹³)₃, ═O or —OC(R¹⁶)₂C(R¹⁶)₂O—; R⁷ and R⁸ independently are —C(R¹⁰)₂— wherein both R¹⁰ are —H or one R¹⁰ is α-OH—, β-OH, α-ester, or β-ester and the other R¹⁰ is —H; R⁹ is —C(R¹⁰)₂—, wherein one R¹⁰ is α-OH, β-OH, α-ester or β-ester and the other R¹⁰ is —H; R¹¹, R¹² and R¹³ independently are optionally substituted C₁₋₆ alkyl or optionally substituted aryl or each R¹², independently selected, is —CH₃ or phenyl, two of R¹³ in each —OSi(R¹³)₃, independently selected, are —CH₃ or —CH₂CH₃ and the remaining R¹³ is —CH₃, —CH₂CH₃, t-butyl or phenyl; and R¹⁶ independently are —H or C₁₋₄ alkyl or two of R¹⁶ and the carbon(s) to which they are attached form a cycloalkyl, optionally C₃, C₅ or C₆ cycloalkyl, and the remaining R¹⁶ are —H.

26A. The process of claim 24A wherein (i) R⁷ and R⁸ are —CH₂—, (ii) R⁷ is —CH(α-OH)— or —CH(β-OH)— and R⁸ is —CH₂— or (iii) R⁷ is —CH₂— and R⁸ is —CH(β-OH)—; R⁹ is —CH(α-OH); the optionally substituted C₁₋₆ alkyl of each R¹¹, independently selected, is —CH₃ or —CH₂CH₃; each R¹², independently selected, is —CH₃ or phenyl; two of R¹³ in each —OSi(R¹³)₃, independently selected, are —CH₃ or —CH₂CH₃ and the remaining R¹³ is —CH₃, —CH₂CH₃, t-butyl or phenyl.

27A. The process of embodiment 26A wherein R¹² and R¹³ are —CH₃ or R¹² is —CH₃ and two of R¹³ are —CH₃ or —CH₂CH₃ and the remaining R¹³ is —CH₂CH₃, t-butyl or phenyl.

28A. The process of embodiment 24A, optionally after protecting group removal, wherein the 3α-O-linked-5α-androstane steroid prepared has the structure

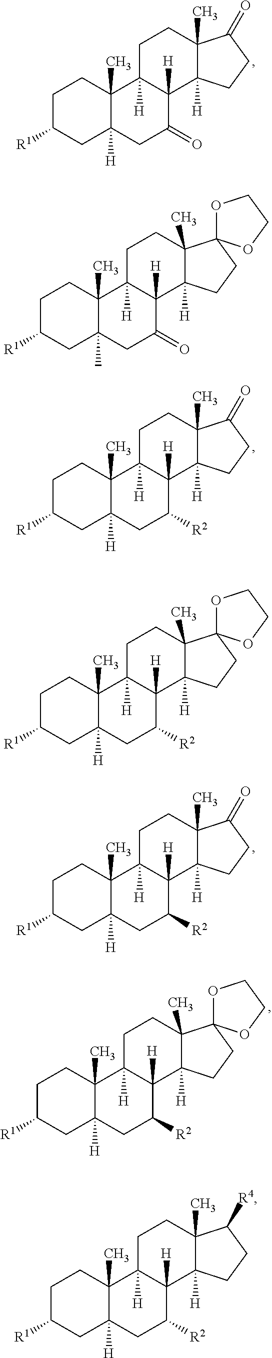

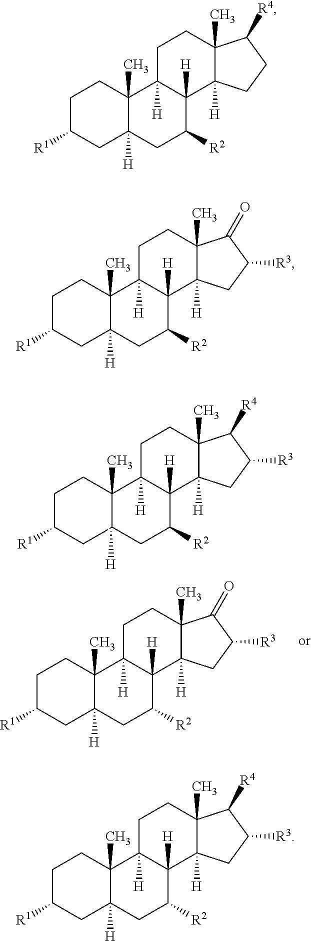

29A. The process of embodiment 24A wherein the 3α-O-linked-5α-androstane steroid prepared is 5α-androstan-7,17-dione-3α-ol, 3α-acetoxy-5α-androstan-7,17-dione, 17,17-ethylenedioxy-5α-androstan-7-one-3α-ol, 17,17-ethylenedioxy-3α-acetoxy-5α-androstan-7-one, 5α-androstan-17-one-3α,7α-diol, 17,17-ethylenedioxy-5α-androstane-3α,7α-diol, 5α-androstan-17-one-3α,713-diol, 17,17-ethylenedioxy-5α-androstane-3α,713-diol, 5α-androstane-3α,7α,17β-triol, 5α-androstane-3α,7β,17β-triol, 5α-androstane-3α,7α,16α,17β-tetrol, 5α-androstane-3α,7β,16α,17β-tetrol, 16α-fluoro-5α-androstane-3α,76,1713-triol, 16α-methoxy-5α-androstane-3α,7β,17β-triol, 16α-methyl-5α-androstane-3α,7β,17β-triol or 16α-propyl-5α-androstane-3α,7β,17β-triol. In other embodiments the 3α-O-linked-5α-androstane compound is any of one these enumerated compounds represented by the formula of embodiment 25A wherein one or more additional suitable mono-O-linked substituents such as —OR^{PR}, —OTMS, OTBDMS or acetoxy are present independently in R⁷, R⁸ and R⁹. Preferred are those compounds additionally having one of R⁷, R⁸, R⁹ as —C(R¹⁰)₂— wherein R¹⁰ in the α- or β-configuration is —OR^{PR}, —OTMS, OTBDMS or acetoxy and the other R¹⁰ is —H

30A. The process of embodiment 21A further comprising the step of (6a) contacting a suitably protected 3α-O-linked-androst-5-ene, obtained or prepared from the 3α-O-linked-androst-5-ene product from the process of claim 21A having a ═O moiety (ketone) at position C-17 with a suitably protected optionally substituted alkyl, optionally substituted alkenyl or optionally substituted alkynyl organometallic anion, wherein the organometallic anion adds to the ═O moiety; wherein a 3α-O-linked androst-5-ene steroid product having disubstitution at position C-17 is prepared.

31A. The process of embodiment 29A further comprising the step of (7a) contacting the initial oxyanion addition product resulting from step (6a) of claim 30A with an electrophile having the structure R¹¹-LG, R¹²—C(O)-LG, (R¹³)₃Si-LG or (R¹⁴)₂N—C(O)-LG, wherein LG is a leaving group and R¹¹, R¹², R¹³ and R¹⁴ are a suitable monovalent C-linked moiety, independently selected; wherein the monovalent O-linked moiety of R⁴ so obtained is an ester, an ether, a silyl ether or a carbamate derived from the electrophile of step (7a) and the other R⁴ is the optionally substituted alkyl, optionally substituted alkenyl or optionally substituted alkynyl derived from the organometallic anion of step (6a) of embodiment 30A. Preferred LG moities include —F, —Cl, —Br, —I, benzenesulfonate, p-toluenesulfonate, triflate and N-hydroxysuccinate.

32A. The process of embodiment 30A wherein the organometallic anion has the structure of M-C≡C—Si(R¹³)₃, wherein R¹³ independently are C₁₋₆ alkyl or aryl or R¹³ are —CH₃; and wherein M represents a Group I, Group II or transition metal in its appropriate oxidate state. Preferred metals are Na, Li, Mg or Zn with Na and Li particularly preferred.

33A. The process of embodiment 30A wherein the C17-disubstituted 3α-O-linked androst-5-ene steroid prepared, optionally after protecting group removal, has the structure

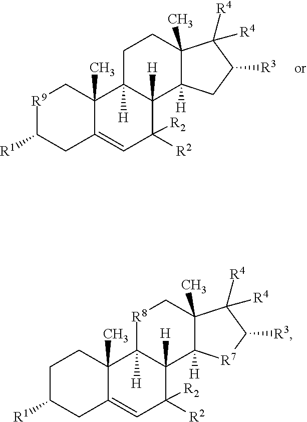

wherein R¹ is —OH, —OR^{PR}, —OR¹¹, —OC(O)—R¹² or —OSi(R¹³)₃; one of R² is —OH, —OR^{PR}, —OR¹¹, —OC(O)—R¹² or —OSi(R¹³)₃ and the other R² is —H or both R² together are ═O; R³ is —H, —OH, —OR^{PR}, —OR¹¹, —OC(O)—R¹², fluoro or optionally substituted alkyl; one R⁴ is —OH, —OR¹¹, —OC(O)—R¹², —OSi(R¹³)₃ and the other R⁴ is an optionally substituted alkynyl, wherein the optionally substituted alkynyl has the structure —C≡R, wherein R is CR^{A} and wherein R^{A} is H, halogen or optionally substituted alkyl or —Si(R¹³)₃; wherein (i) R¹¹, R¹² and R¹³ independently are optionally substituted C₁₋₆ alkyl or optionally substituted aryl or (ii) each R¹¹, independently selected, is —CH₃ or —CH₂CH₃, each R¹², independently selected, is —CH₃ or phenyl and two of R¹³ in each —OSi(R₁₃)₃, independently selected, are —CH₃ or —CH₂CH₃ and the remaining R¹³ are —CH₃, —CH₂CH₃, t-butyl or phenyl; wherein R⁷, R⁸ and R⁹ independently are —C(R¹⁰)₂, wherein R¹⁰ are as previously described in embodiment 25A. In these embodiments preferred halogen and optionally substituted alkyl groups for R^{A} are -chloro, methyl, CH₂OH and CH₂OR^{PR}.

34A. The process of embodiment 30A wherein the C17 di-substituted 3α-O-linked androst-5-ene steroid prepared, optionally after protecting group, removal has the structure

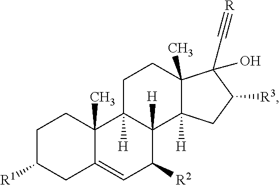

wherein R¹ and R² independently are —OH or —OSi(R¹³)₃; R³ is —H, —OH or —OSi(R¹³)₃ and R in —C≡R is CR^{A} wherein R^{A} is —H, optionally substituted C₁₋₆ alkyl or —Si(R¹³)₃; wherein (i) R¹³ independently are C₁₋₆ alkyl or aryl or (ii) two of R¹³ in one or more of —OSi(R¹³)₃ or in —Si(R¹³)₃ are —CH₃ or —CH₂CH₃ and the remaining R¹³ are —CH₃, —CH₂CH₃, t-butyl or phenyl, independently selected.

35A. The process of embodiment 34A wherein R¹ and R² independently are —OH or ≦OSi(R¹³)₃ wherein R¹³ are —CH₃; R³ is —H and R^{A} is —Si(CH₃)₃.

36A. The process of embodiment 30A wherein the 3α-O-linked androst-5-ene steroid prepared is 17α-ethynyl-androst-5-ene-3α,7β,17β-triol, 17α-ethynyl-androst-5-ene-3α,7α,17β-triol, 17α-ethynyl-androst-5-ene-3α,76,16α,17β-tetrol, 17α-ethynyl-androst-5-ene-3α,7α,16α,17β-tetrol, 17α-ethenyl-androst-5-ene-3α,7β,17β-triol, 17α-methyl-androst-5-ene-3α,76,16α,17β-tetrol, 17α-ethynyl-16α-fluoro-androst-5-ene-3α,7β,17β-triol or 17α-ethynyl-16α-methoxy-androst-5-ene-3α,7β,17β-triol. In other embodiments the C17-disubstituted androst-5-ene compound is any of one these enumerated compounds represented by the formula of embodiment 33A wherein one or more additional suitable mono-O-linked substituents such as —OR^{PR}, —OTMS, —OTBDMS or acetoxy are present independently in R⁷, R⁸ and R⁹. Preferred are those compounds additionally having one of R⁷, R⁸, R⁹ as —C(R¹⁰)₂— wherein R¹⁰ in the α- or β-configuration is —OR^{PR}, —OTMS, OTBDMS or acetoxy and the other R¹⁰ is —H.

37A. The process of embodiment 30A further comprising the step of (6b) contacting a suitably protected 3α-O-linked-5α-androstane, obtained or prepared from the 3α-O-linked-androst-5-ene product from the process of claim 30A, having a ═O moiety (ketone) at position C-17 with a suitably protected optionally substituted alkyl, optionally substituted alkenyl or optionally substituted alkynyl organometallic anion; wherein the organometallic anion adds to the ═O moiety; wherein a 3α-O-linked 5α-androstane steroid product having disubstitution at position C-17 is prepared.

38A. The process of embodiment 37A further comprising the step of (7b) contacting the initial oxyanion addition product resulting from step (6b) of claim 37A with an electrophile having the structure R¹¹-LG, R¹²—C(O)-LG, (R¹³)₃Si-LG or (R¹⁴)₂N—C(O)-LG wherein LG is a leaving group and R¹¹, R¹², R¹³ and R¹⁴ are a suitable monovalent C-linked moiety, independently selected; wherein the monovalent O-linked moiety of R⁴ so obtained is an ester, an ether, a silyl ether or a carbamate is derived from the electrophile of step (7b) and the other R⁴ is the optionally substituted alkyl, optionally substituted alkenyl or optionally substituted alkynyl derived from the organometallic anion of step (6b) of embodiment 37A. Preferred LG moities include —F, —Cl, —Br, —I, benzenesulfonate, p-toluenesulfonate, triflate and N-hydroxysuccinate.

39A. The process of embodiment 38A wherein the organometallic anion has the structure of M-C≡C—Si(R¹³)₃, wherein R¹³ independently are C₁₋₆ alkyl or aryl or R¹³ are —CH₃; wherein M is a Group I, Group II or transition metal or is Na, Li, Mg or Zn.

40A. The process of embodiment 37A wherein the C17-disubstituted 3α-O-linked 5α-androstane steroid prepared, optionally after protecting group removal, has the structure

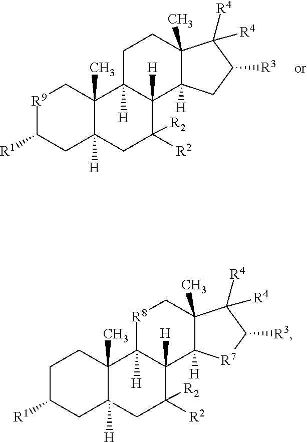

wherein R¹ is —OH, —OR^{PR}, —OR¹¹, —OC(O)—R¹² or —OSi(R¹³)₃; one of R² is —OH, —OR^{PR}, —OR¹¹, —OC(O)—R¹² or —OSi(R¹³)₃ and the other R² is —H or both R² together are ═O; R³ is —H, —OH, —OR^{PR}, —OR¹¹, —OC(O)—R¹², fluoro or optionally substituted alkyl; one R⁴ is —OH, —OR¹¹, —OC(O)—R¹², —OSi(R¹³)₃ and the other R⁴ is an optionally substituted alkynyl wherein the optionally substituted alkynyl has the structure —C≡R; wherein R is CR^{A} and wherein R^{A} is H, optionally substituted alkyl or —Si(R¹³)₃; wherein (i) R¹¹, R¹² and R¹³ independently are optionally substituted C₁₋₆ alkyl or optionally substituted aryl or (ii) each R¹¹, independently selected, is —CH₃ or —CH₂CH₃, each R¹², independently selected, is —CH₃ or phenyl and two of R¹³ in each —OSi(R₁₃)₃, independently selected, are —CH₃ or —CH₂CH₃ and the remaining R¹³ are —CH₃, —CH₂CH₃, t-butyl or phenyl and wherein R⁷, R⁸ and R⁹ independently are —C(R¹⁰)₂— wherein R¹⁰ are as previously described in embodiment 25A.

41A. The process of embodiment 37A wherein the C17 di-substituted 3α-O-linked 5α-androstane steroid prepared, optionally after protecting group, removal has the structure

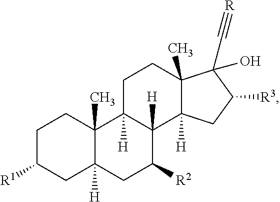

wherein R¹ and R² independently are —OH or —OSi(R¹³)₃; and R³ is —H, —OH or —OSi(R¹³)₃ and R in —C≡R is CR^{A}, wherein R^{A} is —H, optionally substituted C₁₋₆ alkyl or —Si(R¹³)₃; wherein (i) R¹³ independently are C₁₋₆ alkyl or aryl or (ii) two of R¹³ in one or more of —OSi(R¹³)₃ or in —Si(R¹³)₃ are —CH₃ or —CH₂CH₃ and the remaining R¹³ are —CH₃, —CH₂CH₃, t-butyl or phenyl, independently selected.

42A. The process of embodiment 41A wherein R¹ and R² independently are —OH or —OSi(R¹³)₃, wherein R¹³ are —CH₃, R³ is —H and R^{A} is —Si(CH₃)₃.

43A. The process of embodiment 37A wherein the C17 di-substituted 3α-O-linked 5α-androstane steroid prepared is 17α-ethynyl-androst-5-ene-3α,17β-diol, 17α-ethynyl-5α-androstane-3α,17β-diol, 17α-ethenyl-5α-androstane-3α,17β-diol, 17α-ethyl-5α-androstane-3α,17β-diol, 17α-methyl-androst-5-ene-3α,17β-diol, 17α-ethynyl-16α-fluoro-5α-androstane-3α,17β-diol, 17α-ethynyl-16α-methoxy-5α-androstane-3α,17β-diol, 17α-ethynyl-16α-fluoro-androst-5-ene-3α,17β-diol, 17α-ethynyl-androst-5-ene-3α,16α,17β-triol or 17α-ethynyl-5α-androstane-3α,16α,17β-triol. In other embodiments the C17 di-substituted 3α-O-linked 5α-androstane compound is any of one these enumerated compounds represented by the formula of embodiment 40A wherein one or more additional suitable mono-O-linked substituents such as —OR^{PR}, —OTMS, —OTBDMS or acetoxy are present independently in R⁷, R⁸ and/or R⁹. Preferred are those compounds additionally having one of R⁷, R⁸, R⁹ as —C(R¹⁰)₂— wherein R¹⁰ in the α- or β-configuration is —OR^{PR}, —OTMS, OTBDMS or acetoxy and the other R¹⁰ is —H.

44A. A process to prepare a 3α-O-linked-androst-5-ene steroid comprising the step of (1) contacting a suitably protected 3β-hydroxy steroid with an azo-di-carboxylate ester, a tri-substituted phosphine and an organic acid having the structure of R¹²C(O)OH wherein R¹² is C₁₋₆ alkyl, C₃₋₆ cycloalkyl or optionally substituted aryl, wherein the suitably protected 3β-hydroxy steroid has the structure

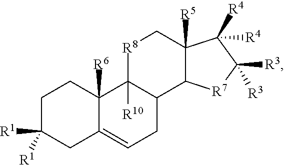

wherein R¹ in the β-configuration is —OH and R¹ in the α-configuration is —H or a suitable optionally substituted alkyl, optionally a C₁₋₄ optionally substituted alkyl such as methyl, ethyl or n-propyl; R³ independently or together are —H, halogen, a suitable C-linked moiety, a suitable monovalent O-linked moiety, ═O (ketone) or —O—C(R¹⁶)₂—C(R¹⁶)₂—O— (ketal); R⁴ in the β-configuration is a suitable monovalent O-linked moiety; R⁴ in the α-configuration is —H or a suitable C-linked moiety or R⁴ together are ═O (ketone) or —O—C(R¹⁶)₂—C(R¹⁶)₂—O— (ketal), wherein R¹⁶ independently are —H or C₁₋₄ alkyl or two of R¹⁶ and the carbon(s) to which they are attached form an optionally substituted C₃, C₅ or C₆ cycloalkyl or C₃, C₅ or C₆ spiroalkyl; R⁵ and R⁶ independently are —H or a suitable optionally substituted alkyl; R⁷ and R⁸ independently are —C(R¹⁰)₂—; wherein R¹⁰ independently or together are —H, a suitable halogen, a suitable monovalent C-linked moiety or a suitable monovalent O-linked moiety or both R¹⁰ together are ═O or —O—C(R¹⁶)₂—C(R¹⁶)₂—O— (ketal); R¹⁰ at position C-9 is —H or halogen; R^{PR} independently are —H or protecting group;

wherein the C-linked moieties are independently a suitable optionally substituted alkyl group, optionally substituted alkenyl group or optionally substituted alkynyl group; and wherein the monovalent O-linked moieties independently are —OR^{PR} an ester or an ether; wherein the molar ratio of the azo-di-carboxylate ester to the 3β-hydroxy steroid is less than 1.5:1 and greater than 1.0:1; wherein a 3α-androst-5-ene product having a 3α-O-linked ester substantially free of 3α,5α-cycloandrostane side-products is obtained.

45A. The process of embodiment 44A further comprising the step of (2) contacting the 3α-O-linked ester androst-5-ene obtained or prepared from the product of claim 44 with a basic solution wherein the 3α-O-ester is converted to 3α-OH.

46A. The process of embodiment 44A wherein the molar ratio of the azo-di-carboxylate ester to the 3β-hydroxy steroid is about 1.3:1.

47A. The process of embodiment 44A wherein the azo-di-carboxylate ester, tri-substituted phosphine and organic acid are in substantially equimolar amounts.

48A. The process of embodiment 44A wherein R¹⁹ of the organic acid is an optionally substituted phenyl wherein the 3α-O-linked ester androst-5-ene obtained or prepared from the product of step (1) of claim 44 is capable of hydrolysis in an aqueous solution at ambient temperature to provide a 3α-hydroxy-androst-5-ene steroid.

49A. The process of embodiment 44A wherein the an azo-di-carboxylate ester is added to a mixture of the tri-substituted phosphine, organic acid and p-hydroxy steroid at between about 0 to 25° C.

50A. The process of embodiment 44A wherein the azo-di-carboxylate ester is added to a mixture of the tri-substituted phosphine at a temperature of between about 0-10° C. whereupon the mixture is warmed to between about 10-25° C.

51A. The process of embodiment 44A wherein R¹⁹ is p-NO₂-phenyl and the azo-di-carboxylate ester has the structure R¹⁹OC(O)N═NC(O)OR¹⁹ wherein R¹⁹ is —CH₂CH₃ (DEAD) or —CH(CH₃)₂ (DIAD).

52A. The process of embodiment 44A wherein 3α-O-linked-androst-5-ene steroid prepared, optionally after protecting group removal, has the structure

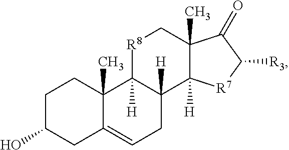

wherein R³ is —H, halogen, a monovalent O-linked moiety or a monovalent C-linked moiety; R⁷ and R⁸ independently are —C(R¹⁰)₂ wherein R¹⁰ independently are —H a monovalent O-linked moiety or a monovalent C-linked moiety.

53A. The process of embodiment 44A wherein 3α-O-linked-androst-5-ene steroid prepared is androst-5-en-17-one-3α-ol (3α-DHEA), androst-5-en-17-one-3α,116-diol, androst-5-en-17-one-3α,15α-diol, androst-5-en-17-one-3α,15α,16α-triol, androst-5-en-17-one-3α,11β,16α-triol, 16α-fluoro-androst-5-en-17-one-3α-ol. In other embodiments the 3α-O-linked-androst-5-ene compound is any of one these enumerated compounds represented by the formula of embodiment 52A wherein one or more additional suitable mono-O-linked substituents such as —OR^{PR}, —OTMS, —OTBDMS or acetoxy are present independently in R⁷ and/or R⁸. Preferred are those compounds additionally having one of R⁷, R⁸ as —C(R¹⁰)₂— wherein R¹⁰ in the α- or β-configuration is —OR^{PR}, —OTMS, —OTBDMS or acetoxy and the other R¹⁰ is —H.

54A. The process of embodiment 44A further comprising the step of (3) contacting a suitably protected 3α-O-linked androst-5-ene prepared or obtained from the 3α-O-linked-androst-5-ene product of embodiment 44A with a hydrogen donor to reduce the Δ⁵ functional group, wherein a 3α-O-linked-5α-androstane product is obtained.

55A. The process of embodiment 44A or 54A further comprising the step of (4) contacting a suitably protected 3α-O-linked-androst-5-ene obtained or prepared from the 3α-O-linked-androst-5-ene product, having a ═O moiety (ketone) at position C17 of embodiment 44A or a suitably protected 3α-O-linked-5α-androstane obtained or prepared from the 3α-O-linked-5α-androstane steroid product of embodiment 54A, having a ═O moiety (ketone) at position C17, with a suitably protected optionally substituted alkyl, an optionally substituted alkenyl or an optionally substituted alkynyl organometallic anion, wherein the organometallic anion adds to the ═O moiety to provide a 3α-O-linked 5α-androstane product or a 3α-O-linked 5α-androstane product having disubstitution at position C17.

56A. The process of embodiment 55A further comprising the step of (5) contacting the initial oxyanion addition product resulting from step (4) of claim 55A with an electrophile having the structure R¹¹-LG, (u) LG, (R¹³)₃Si-LG or (R¹⁴)₂N—C(O)-LG wherein LG is a leaving group and R¹¹, R¹², R¹³ and R¹⁴ are a suitable monovalent C-linked moiety, independently selected; wherein a 3α-O-linked 5α-androstane product or a 3α-O-linked androst-5-ene steroid product having disubstitution at position C-17 is prepared, wherein one C-17 substituent is a monovalent O-linked moiety, wherein the monovalent O-linked moiety is —OH or an ester, an ether, silyl ether or a carbamate derived from the electrophile of step (5) and the other C17 substituent is the optionally substituted alkyl, optionally substituted alkenyl or optionally substituted alkynyl of step derived from the organometallic anion of step (4) of embodiment 55A. Preferred LG moities include —F, —Cl, —Br, —I, benzenesulfonate, p-toluenesulfonate, triflate and N-hydroxysuccinate.

57A. The process of embodiment 55A wherein the organometallic anion has the structure of M-C≡C—Si(R¹³)₃ wherein R¹³ independently are C₁₋₆ alkyl or aryl and M is a Group I, Group II or transition metal or is Na, Li, Mg or Zn.

58A. The process of embodiment 55A wherein the 3α-O-linked androst-5-ene steroid or the 3α-O-linked 5α-androstane steroid prepared, optionally after protecting group removal, is 17α-ethynyl-androst-5-ene-3α,17β-diol, 17α-ethynyl-5α-androstane-3α,17β-diol, 17α-ethenyl-5α-androstane-3α,17β-diol, 17α-ethyl-5α-androstane-3α,17β-diol, 17α-methyl-androst-5-ene-3α,17β-diol, 17α-ethynyl-16α-fluoro-5α-androstane-3α,17β-diol, 17α-ethynyl-16α-methoxy-5α-androstane-3α,17β-diol, 17α-ethynyl-16α-fluoro-androst-5-ene-3α,17β-diol, 17α-ethynyl-androst-5-ene-3α,16α,17β-triol or 17α-ethynyl-5α-androstane-3α,16α,17β-triol. In other embodiments the 3α,4α-epoxy-androst-5-ene compound is any of one these enumerated compounds represented by the formula of embodiment 44A or 52A wherein one or more additional suitable mono-O-linked substituents such as —OR^{PR}, —OTMS, OTBDMS or acetoxy are present independently in R⁷ and/or R⁸. Preferred are those compounds additionally having one of R⁷, R⁸ as —C(R¹⁰)₂— wherein R¹⁰ in the α- or β-configuration is —OR^{PR}, —OTMS, —OTBDMS or acetoxy and the other R¹⁰ is —H.

Variations and modifications of these embodiments and other portions of this disclosure will be apparent to the skilled artisan after a reading thereof. Such variations and modifications are within the scope of this invention. The claims in this application or in applications that claim priority from this application will more particularly describe or define the invention. All citations or references cited herein are incorporated herein by reference in their entirety.

### EXAMPLES

### Example 1

The following describes inversion of configuration at position C3 of a 3β-hydroxy steroid to provide a 3α-hydroxy steroid by Method A.

### Step A. 17,17-Ethylenedioxy-androst-3,5-dien-7-one (6a)

A mixture of compound 5a (30 g, 0.0871 mol), p-toluenesulfonic acid monohydrate (0.384 g, 0.002 mol) and ethylene glycol (18 mL, 0.327 mol) in toluene (80 mL) was refluxed for 8 hr with a Dean and Stork apparatus for removal of water. After cooling, the organic solution was washed with saturated sodium bicarbonate aqueous solution, brine, and dried over magnesium sulfate. The solvent was removed under reduced pressure. The residue was further dried in vacuo to give 6a as a pale yellow solid. (98% yield). Selected ¹H NMR data: (CDCl₃, ppm): δ 6.18 (m, 1H), 6.10 (dd, 1H, J=9.4 Hz, 2.0 Hz), 5.60 (s, 1H), 3.91 (m, 2H), 3.86 (m, 2H), 1.16 (s, 3H), 0.90 (s, 3H).

### Step B. 3α,4α-Epoxy-17,17-ethylenedioxy-androst-5-en-7-one (7a)

To the stirring solution of compound 6a (2.84 g, 8.65 mmol) prepared from Step A in 20 mL of chloroform was added a solution of m-chloroperoxy benzoic acid (0.0088 mol) in chloroform (20 mL). The reaction mixture was stirred at room temperature. After 16 hr., another portion of m-chloroperoxy benzoic acid (2 mmol) was added, and the reaction mixture was stirred for additional 10 h. The mixture was under reduced pressure to remove most of the volume of solvent. Diethyl ether and sodium sulfite aqueous solution were added and the mixture was stirred at room temperature for 1 h. The organic layer was separated, and the aqueous layer was extracted with ether and combined organic layers were washed with 1 N sodium hydroxide aqueous solution, dried over magnesium sulfate and concentrated in vacuo to give 7a as a white crude product (2.7 g), which was carried on next step without further purification. Selected ¹H NMR data of a purified sample (CDCl₃, ppm): δ 6.04 (s, 1H), 3.92 (m, 2H), 3.84 (m, 2H), 3.47 (m, 1H), 3.42 (d, 1H, J=4.1 Hz), 1.08 (s, 3H), 0.87 (s, 3H).

### Step C. 17,17-Ethylenedioxy-androst-5-en-7-one-3α-ol (8a)

A mixture of 7a (1.37 g, 3.97 mmol), denatured ethanol (40 mL), ethyl acetate (8 mL), potassium carbonate (552 mg, 4.0 mmol) and 120 mg (0.056 mmol) of 5% palladium on charcoal was shaken at room temperature under hydrogen (22 psi) on a Parr Shaker for 40 minutes. The reaction mixture was filtered through Celite and the Celite rinsed with 40 ml of dichloromethane. The combined filtrates were concentrated under reduced pressure to give a solid, which was purified by flash chromatography on silica gel, eluted with 1:1 ethyl acetate:hexanes to afford compound 8a (760 mg) as a white solid. Selected ¹H NMR data (CDCl₃, ppm): δ 5.73 (d, 1H, J=1.2 Hz), 4.05 (m, 1H), 3.92 (m, 2H), 3.84 (m, 2H), 2.67 (dt, J=15.0, 3.0 Hz, 1H), 2.46 (m, 1H), 1.21 (s, 3H), 0.87 (s, 3H). Melting Point: 170-173° C.

### Step D. 3α-Hydroxy-androst-5-en-7,17-dione (9a)

To a solution of compound 8a (42 mg, 0.12 mmol) in 3 mL of tetrahydrofuran, 1 mL of acetone and 0.2 mL of water was added 1 N hydrochloric acid solution until a pH of 1-2 was achieved. The reaction mixture was stirred at room temperature for 2 h. The reaction mixture was neutralized by the addition of sodium bicarbonate. The solid was filtered and washed with methanol. The combined filtrates were concentrated in vacuo to give a solid, which was recovered from methanol and water to afford the title compound 5 (32 mg) as a white solid. Selected ¹H-NMR data: (CD₃OD, ppm): δ 5.72 (d, 1H, J=1.2 Hz), 4.13 (m, 1H), 2.73 (m, 1H), 270 (d t, J=15.0, 3.0 Hz, 1H), 2.53 (t, J=11.0 Hz, 1H), 1.27 (s, 3H), 0.91 (s, 3H) ppm. Mp: 253-255° C.

### Example 2

The following describes inversion of configuration at position C-3 of a 3β-hydroxy steroid to provide a 3α-hydroxy steroid by Method B.

### Step A. 3α-(p-Nitrophenylcarbonyloxy)-androst-5-en-17-one (11a)

To a 200 mL flask was added 2 g (100 mol %) of DHEA (10a), 1.28 g (110 mol %) of p-nitrobenzoic acid, 2 g (110 mole %) of triphenylphosphine and 50 mL anhydrous THF. The reaction mixture was stirred until all solids dissolved and then cooled to 4° C. in an ice-water bath. A 3.5 mL (110 mol %) solution of 40% DEAD in anhydrous toluene was added dropwise, whereupon the reaction mixture was warmed to ambient temperature and stirred overnight. The mixture was concentrated in vacuo and the resulting residue was suspended in 5 mL of EtOAc. The solids were then collected by filtration and washed with EtOAc to provide 2.1 g of crude material. Purification from 100 mL MeOH gave 1.1 g of 11a. ¹H-NMR (CDCl₃, ppm): δ 8.28 (d, 2H), 8.15 (d, 2H), 5.37 (d, 1H), 5.30 (s, 1H), 2.49 (d, 1H), 2.45 (q, 1H), 2.41 (d, 1H), 0.89-2.2 (m, 16H), 1.10 (s, 3H), 0.91 (s, 3H).

### Step B. 3α-Hydroxy-androst-5-ene-17-one (3α-DHEA)

To a 50 mL flask was added 0.6 g of 11a, 20 mL of THF, 10 mL of MeOH and 0.27 g NaOH in 1 mL of water. The mixture was stirred at 40° C. for 30 min. and then at ambient temperature for 30 min. Afterwards, the solution was concentrated in vacuo and water was added to form a precipitate. The solids were collected by filtration and dried under vacuum to provide 0.3 g of 3α-DHEA (12a).

### Example 3

The following describes introduction of C17-disubstitution to a 3α-hydroxy steroid.

### Step A. 3α-(Trimethylsilyl)oxy-androst-5-en-17-one (TMS-3α-DHEA)

3α-DHEA (12a) was combined with 1,1,1,3,3,3-hexamethyldisilazane (HMDS) and saccharin (as catalyst) in acetonitrile. The reaction mixture was heated to reflux for several hours with stirring under a nitrogen atmosphere. Liberated ammonia was purged under slight vacuum. The volume was then reduced by distillation, followed by cooling the mixture and collecting the precipitated product by filtration. The filter cake of TMS-3α-DHEA product was washed with cold acetonitrile and dried with warm nitrogen to provide TMS-3α-DHEA (13).

### Step B. 17α-Ethynyl-androst-5-ene-3α,17β-diol (14)

n-Butyl lithium was added slowly to Me₃Si—C≡CH in THF under a nitrogen atmosphere at approximately 0° C. to produce the lithium acetylide Me₃Si—C≡C—Li. The temperature was raised to about 20° C., and TMS-3α-DHEA (13) was added as a solution in THF, and stirred for about 3 hours. The reaction was quenched by raising the temperature to about 40° C. followed by the slow addition of methanol. Liberated acetylene was purged under slight vacuum. Concentrated KOH was then slowly added until gas evolution subsides, and the volume is reduced by approximately 50% by vacuum distillation at approximately 45° C. Excess 6 N HCl was slowly added, while maintaining the temperature at approximately 40° C. The reaction mixture was diluted with water and chilled to approximately 5° C. before collecting the product by filtration and washing the filter cake with cold 50/50 methanol water. The product was dried with warm nitrogen to provide 14. ¹H-NMR (CD₃OD, ppm): δ 5.30 (d, 1H), 3.95 (s, 1H), 2.88 (s, 1H), 2.53 (d, 1H), 2.19 (m 1H), 2.09 (d, 1H), 1.05-2.00 (m, 16H), 1.07 (s, 3H), 0.89 (s, 3H).

### Example 4

The following describes introduction of an O-linked moiety to a 3α-hydroxy steroid at position C-7.

### Step A. 17,17-Ethylenedioxy-3α-acetoxy-androst-5-en-7-one (16)

A 500 L reactor was charged with 200 Kg ethyl acetate and 25 kg of 17,17-ethylenedioxy-3α-acetoxy-androst-5-ene (15), prepared from acetylation and ketalization of 3α-DHEA (12a). The mixture was stirred for 30 minutes whereupon 55 kg of 70% t-butyl peroxide and 9 kg of sodium bicarbonate was added. The reaction mixture was then cooled to 0° C. and 116 kg of 13% sodium perchlorate (aq.) was added over 10 hours so that a reaction temperature below 5° C. and pH between about 7.5 to 8.5 was maintained. After the reaction was complete, the organic layer was separated and the aqueous phase was extracted with ethyl acetate (35 kg×2). The combined organic phase are combined with a solution 33 kg of sodium sulfite in 167 kg of water, and the resulting mixture was stirred at 40° C. for about 3 hours. The organic phase was washed with 50 kg of brine and concentrated to 55-60 Kg whereupon 50 Kg of methanol was added. After refrigeration overnight, the precipitate was filtered, washed with 10 kg of methanol, and dried at 40-50° C. to yield the title compound. ¹H-NMR (CDCl₃, ppm): δ 5.67 (s, 1H), 5.12 (s, 1H), 3.8-4.0 (m. 4H), 2.60 (d, 1H), 2.47 (t, 1H), 2.46 (d, 1H), 2.27 (t, 1H), 2.01 (s, 3H), 1.26-2.05 (m, 14H), 1.21 (s, 3H), 0.88 (s, 3H).

### Step B. 17,17-Ethylenedioxy-3α-acetoxy-androst-5-ene-7β-ol (17)

A 500 L reactor was charged with 48 Kg of THF, 10 kg of 16 and a solution of 9.6 kg CeCl₃.7H₂O in 95 kg methanol. This mixture was cooled to 0° C. whereupon 2.0 Kg of NaBH₄ was added in batches over 3 hours to maintain the temperature below 5° C. After stirring for 30 more minutes, 28 Kg of acetone was added slowly to maintain the temperature below 5° C., with stirring continued for another 30 minutes. To the mixture was added 240 Kg water with stirring continued for 1 hour. The organic solvents were removed under vacuum and the residue was extracted with ethyl acetate (100 Kg+50 Kg). The combined organic phase was washed with brine. Solvent was then removed to provide the title compound. ¹H-NMR (CDCl₃, ppm): δ 5.22 (s, 1H), 5.01 (s, 1H), 3.8-4.0 (m, 5H), 2.47 (d, 1H), 2.27 (d, 1H), 2.02 (s, 3H), 1.15-2.10 (m, 15H), 1.06 (s, 3H), 0.87 (s, 3H).

### Step C. Androst-5-en-17-one-3α,713-diol (18)

The ketal protecting group at position C-17 of the product from Step B was removed using acetone and p-toluenesulfonic acid, followed by hydrolysis of the acetate protecting group with aqueous Na₂CO₃ to provide androst-5-en-17-one-3α,7β-diol. ¹H-NMR (CDCl₃, ppm): δ 5.27 (s, 1H), 4.01 (t, 1H), 3.93 (d, 1H), 2.57 (d, 1H), 2.41 (dd, 1H), 1.21-2.30 (m, 15H), 1.06 (s, 3H), 0.90 (s, 3H).

### Example 5

The following describes introduction of an O-linked substituent to position C-16 of a 3α-hydroxy steroid by way of a bromo intermediate.

### 16α-Bromo-androst-5-en-17-one-3α-ol (19)

A solution of 3α-DHEA (17.8 g, 61.7 mmol) in methanol (1.35 L) was refluxed with copper (II) bromide (36.4 g, 163 mmol) with stirring for 19 hours. To the cooled reaction mixture was added water (1.35 L) and dichloromethane (1.5 L). The organic layer was filtered through anhydrous sodium sulfate and the product recovered from methanol (16.7 g, 45.5 mmol, 74%). Mp 195-207° C. ¹H-NMR (CDCl₃, ppm): δ 5.43 (d, 1H), 4.54 (d, 1H), 4.04 (s, 1H), 2.57 (d, 1H), 1.42-2.30 (m, 15H), 1.22 (t, 1H), 1.04 (s, 3H), 0.92 (s, 3H).

### 3α,16α-Diacetoxy-5-androsten-17-one (21)

To a solution of 19 (12.0 g, 32.7 mmol) in pyridine (1.032 L) and water (0.247 L) in air was added aqueous 1N sodium hydroxide (90 mL) and the mixture was stirred at room temperature for 15 minutes. The reaction mixture was added to an ice/water mixture containing 1.2 L of 1N hydrochloric acid. After saturating with sodium chloride, the solution was extracted with ethyl acetate (2×1 L). The combined organic layers were washed with brine (250 mL), filtered through anhydrous sodium sulfate and concentrated. The crude 5-androstene-3α,16α-diol-17-one (20) was treated with excess acetic anhydride in pyridine at room temperature overnight and purified by column to give 13 (7.46 g, 19.2 mmol, 59%) from methanol. Mp 172.7-173.7° C. ¹H-NMR (CDCl₃, ppm): δ 5.44 (d, 1H), 5.30 (s, 1H), 5.00 (s, 1H), 2.47 (d, 1H), 2.24 (d, 1H), 2.11 (s, 3H), 2.01 (s, 3H), 1.10-2.20 (m, 15H), 1.04 (s, 3H), 1.00 (s, 3H). Mp 172.7-173.7° C. Mp 172.7-173.7° C.

### Example 6

The following describes introduction of an O-linked substituent to position C-7 of a 3α-hydroxy steroid having an O-linked substituent at position C-16.

### 3α,16α-Di-acetoxy-androst-5-ene-17β-ol (22)

To a solution of the diacetate 21 (7.46 g, 19.2 mmol) in dichloromethane (45 mL) and methanol (120 mL) at 0° C. was added sodium borohydride (950 mg). The solution was stirred at 0° C. for 1 hour. After addition of excess acetic acid the reaction mixture was partitioned between dichloromethane and water. The organic layer was filtered through anhydrous sodium sulfate and concentrated to yield a mixture of the 17α (minor) and 17β (major) epimers. This mixture was purified by flash chromatography (25% ethyl acetate in hexanes) to give 6.1 g (15.6 mmol, 81%) of the 17β epimer 22. ¹H-NMR (CDCl₃, ppm): δ 5.70 (s, 1H), 4.98 (s, 1H), 4.82 (m, 1H), 3.52 (d, 1H), 2.47 (d, 1H), 2.25 (d, 1H), 2.12 (s, 3H), 2.04 (s, 3H), 1.10-2.10 (m, 15H), 1.04 (s, 3H), 1.00 (s, 3H). Mp 126.9-128.6° C. The triacetate 3α,16α,17β-tri-acetoxy-androst-5-ene-17β-ol (23) was prepared by treating 22 with excess acetic anhydride in pyridine at room temperature overnight. Purification by column provided 6.0 g 23 (13.9 mmol, 89%). ¹H-NMR (CDCl₃, ppm): δ 5.28 (m, 2H), 4.98 (s, 1H), 4.56 (d, 1H), 2.50 (d, 1H), 2.32 (m, 1H), 2.22 (d, 1H), 2.08 (s, 3H), 2.06 (s, 3H), 2.05 (s, 3H), 0.90-1.90 (m, 14H), 1.06 (s, 3H), 0.92 (s, 3H).

### 3α,16α,17β-tri-acetoxy-androst-5-en-7-one (24)

A solution of the triacetate 23 (6.0 g, 13.9 mmol) in benzene (255 mL) was treated with celite (25.5 g), pyridinium dichromate (31.5 g) and 70% tert-butyl hydrogen peroxide (9.0 mL) and stirred at room temperature for 19 hours. Anhydrous diethyl ether (255 mL) was added and reaction mixture was cooled in an ice bath for 1 hour. The resulting solid was filtered off and washed with ether (2×50 mL). The combined organic portions were concentrated and purified by flash chromatography (29% ethyl acetate in hexanes) to give 3.45 g of 23 (7.7 mmol, 55%). ¹H-NMR (CDCl₃, ppm): δ 5.69 (s, 1H), 5.32 (dd, 1H), 5.14 (s, 1H), 4.61 (d, 1H), 3.12 (m, 1H), 2.61 (d, 1H), 2.48 (d, 1H), 2.37 (t, 1H), 2.08 (s, 3H), 2.06 (s, 3H), 2.05 (s, 3H), 1.20-1.90 (m, 11H), 1.06 (s, 3H), 0.90 (s, 3H).

### Androst-5-ene-3α,7α,16α,17β-tetrol (26) and Androst-5-ene-3α,713,16α,17β-tetrol (27)

To a solution of 23 (3.45 g, 7.7 mmol) in dichloromethane (15 mL) and methanol (30 mL) at 0° C. was added sodium borohydride (1.0 g) and the solution was stirred at 0° C. for 2 hours. After addition of excess acetic acid (1.5 mL) the reaction mixture was partitioned between dichloromethane and water. The organic layer was filtered through anhydrous sodium sulfate and concentrated to yield a mixture of the 7α (minor) epimer 3α,16α,17β-tri-acetoxy-androst-5-ene-7α-ol (24) and the 7β (major) epimer 3α,16α,17β-tri-acetoxy-androst-5-ene-7β-ol (25). This mixture was saponified in methanol (100 mL) with 1N sodium hydroxide (60 mL) overnight at room temperature. The crude tetrols were recovered by partitioning the saponification mixture between ethyl acetate and brine. The epimers were separated by HPLC to give 220 mg of 26 (0.68 mmol, 9%) as the minor product, Mp 243-248.3° C.); selected ¹H-NMR peaks (CD₃OD, ppm): δ 0.77 (s, 3H), 1.02 (s, 3H), 2.11 (m, 1H), 2.57 (m, 1H), 3.34 (s, 1H), 3.44 (d, 1H), 3.70 (br t, 1H), 4.04 (m, 2H), 5.55 (dd, 1H) and 27 as the major product, selected ¹H-NMR peaks (CD₃OD, ppm): 5.23 (s, 1H), 4.01 (m, 2H), 3.80 (m, 1H), 3.38 (d, 1H), 2.53 (d, 1H), 2.10 (d, 1H), 2.08 (d, 1H), 1.0-1.9 (m, 15H), 1.04 (s, 3H), 0.77 (s, 3H).

### Example 7

### 17α-ethynyl-5α-androstane-3α,17β-diol (28)

The title compound is prepared according to the following reaction scheme wherein the precursor, 3α-DHEA, is obtained by Method B according to Example 2.

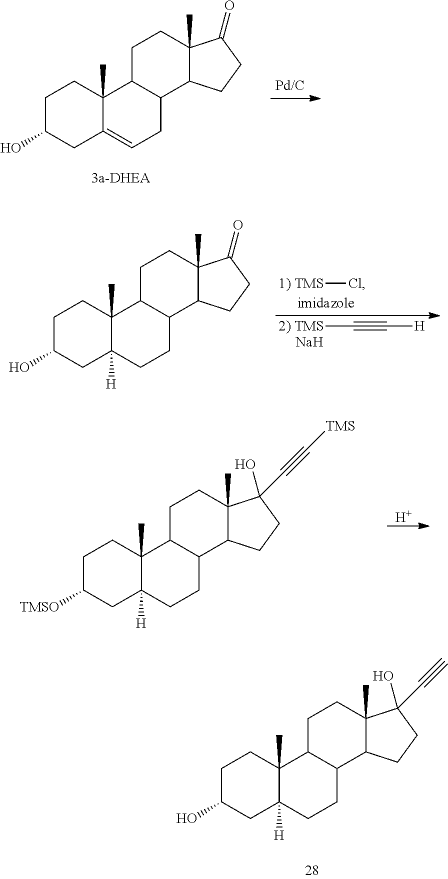

Introduction of the 17α-ethynyl group is provided in Example 3, Step B.

### Example 8

### 17α-ethynyl-androst-5-ene-3α,7β,17β-triol (29)

The title compound is prepared by Method A according to the following reaction scheme wherein the precursor, 3α,4α-epoxy-17,17-ethylenedioxy-androst-5-ene (7a), is prepared according to the procedure of Example 1 (step B). This reaction scheme is a variation of Method A where the first hydrogen donor and second hydrogen donor are identical (e.g., lithium aluminum hydride) so as to reductively open the 3α,4α-epoxy group with concomitant C7-ketone reduction.

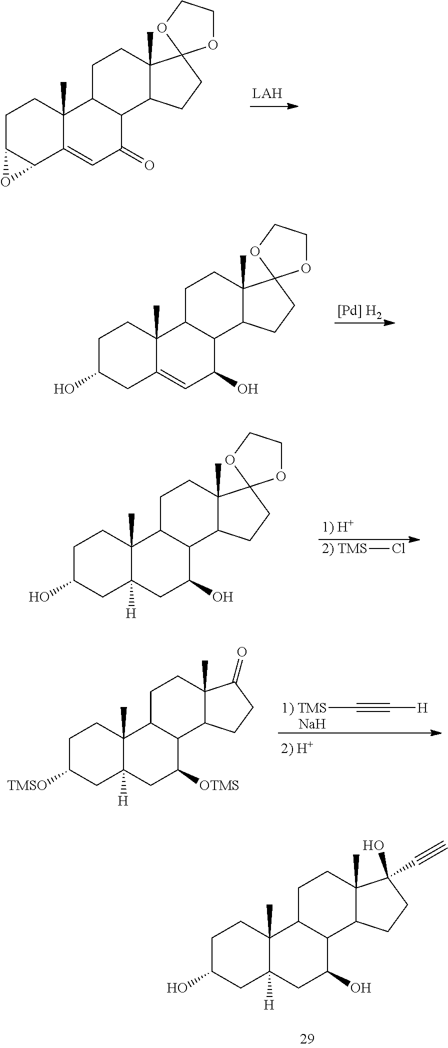

### Example 9

### 17α-ethynyl-5α-androstane-2α,3α,17β-triol (37a) and 17α-ethynyl-5α-androstane-2β, 3α,17β-triol (37b)

The title compounds are prepared according to the following reaction scheme wherein intermediates androst-5-en-17-one-2α,3α-diol (35a) and androst-5-en-17-one-2β,3α-diol (35b) are obtained by Method B.

The requisite suitably protected 2α- or 2β-O-linked-testosterone precursors 32a or 32b, respectively, are prepared from a suitably protected testosterone 30 through a corresponding 6-bromo derivative, which is obtained by contacting 30 with N-bromo-succinimide. The 6-bromo derivative is then contacted with mixture of an organic acid of structure R¹²C(O)OH and its potassium or manganese salt to provide 32 wherein —OR^{PR} is —OC(O)R¹² as its 2α or 2β isomer or a mixture thereof separable by, e.g., standard chromatographic techniques.

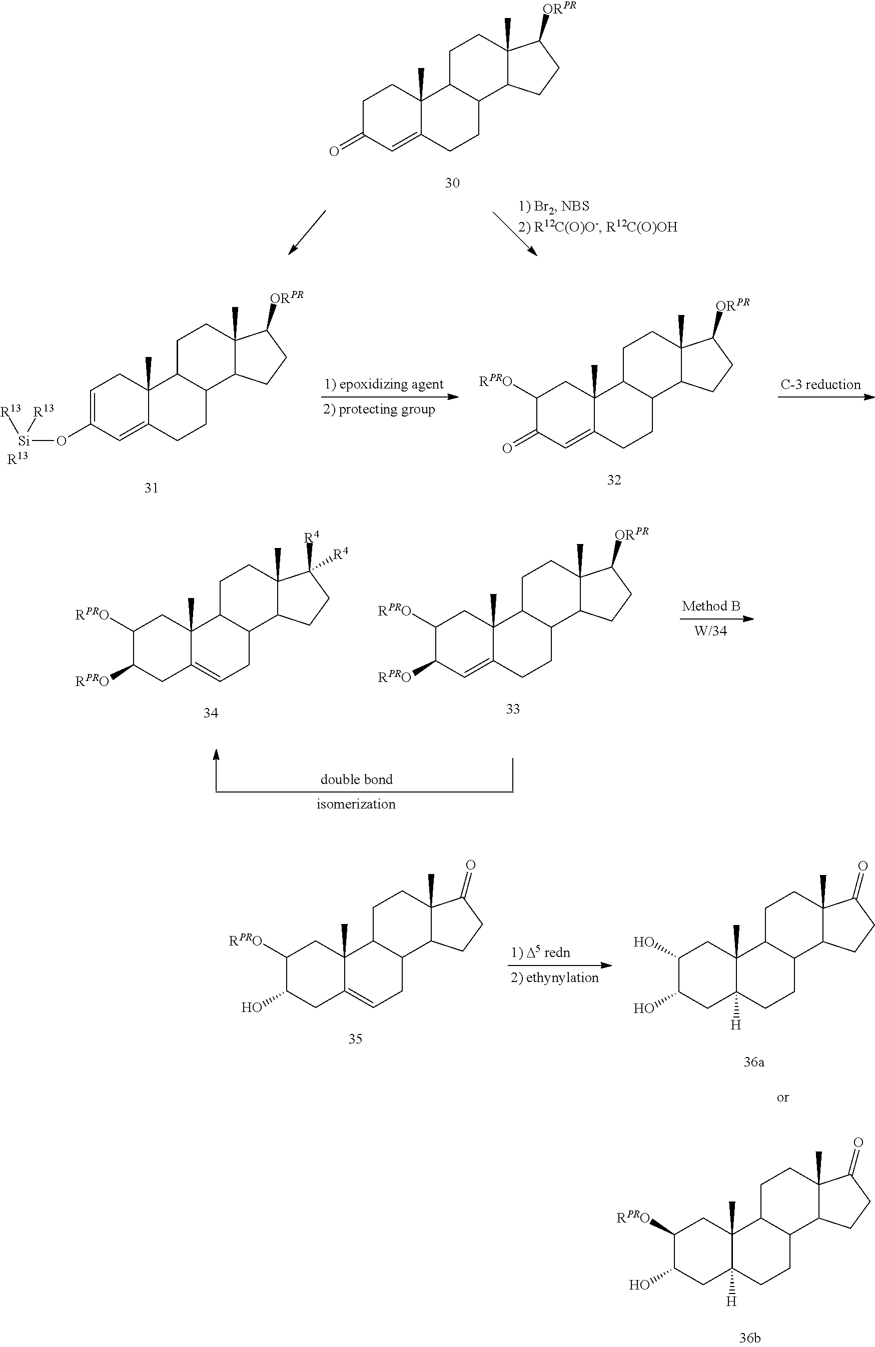

Exemplary reaction conditions for transformation of 30 to 32 are adapted from Baran, J. Amer. Chem. Soc. 80: 1687-1690 (1958), Rosenkranz, J. Amer. Chem. Soc. 77: 145-8 (1955), Fieser, et al. J. Amer. Chem. Soc. 75: 4716 (1953), Rivett, et al. J. Org. Chem. 15: 35-9 (1950), Herran, et al. J. Amer. Chem. Soc. 76: 5531 (1954), Demir, et al. J. Org. Chem. 54(17): 4020-2 (1989), Wiechert, et al. Helv. Chim. Acta 49: 1581-91 (1966), Rao, et al. J. Org. Chem. 28: 270 (1963), Bednardski, et al. J. Med. Chem. 32(1): 202-213 (1989) and in U.S. Pat. Nos. 2,862,939 and 2,948,740, which are specifically incorporated by reference herein.

Alternatively 32 is prepared by contacting a silyl enol ether 31 (wherein R¹³ are independently selected C₁₋₆ alkyl or aryl, preferably —CH₃) derived from 30 with an epoxidizing agent. Exemplary conditions for this alternative route are adapted from Iwata, et al. Tet. Lett. 26(27): 3227-3230 (1985), Rubottom, et al. J. Org. Chem. 43(8): 1599-1602 (1978) Sato, et al. Tet. Lett. 37(34): 6141-4 (1996).

Reduction of the C-3 ketone in 32 is then expected to provide a 2α/β-O-linked-3β-hydroxy-androst-5-ene (34) or -androst-4-ene steroid (33), whose double bond may be isomerized to provide 34, wherein R⁴ in the β-configuration is —OR^{PR} and R⁴ in the α-configuration is —H. Deprotection at C-17 followed by oxidation to the C-17-ketone then provides 34 wherein both R⁴ together are ═O. After protecting group manipulation, a suitably protected 3β-hydroxy-2a/b-O-linked-androst-5-en-17-one steroid of structure 34 is obtained that is subjected to Method B to provide an androst-5-en-17-one-3α,2a/13-diol steroid, e.g., 2α-hydroxy-3α-DHEA (35a) or 2β-hydroxy-3α-DHEA (35b) where —OR^{PR} in 35 is —OH. Compound 35 is then contacted with a hydrogen atom donor to reduce the Δ⁵ functional group as shown in Example 7. Predominate approach by a hydrogen atom donor to the α-face of 35a is expected due to the directing effect of its 2α-hydroxy substituent reinforcing that of the 2α-hydroxy substituent to provide the desired 5α-androstan-17-one-2α,3α-diol steroid 36a. For the 2β-isomer, the preferred —OR^{PR} in 35 is an ester, since steric effects are now expected to reinforce the directing effect of its 2α-hydroxy substituent to also result in predominate approach by a hydrogen atom donor to the α-face, thus giving the desired 5α-androstan-17-one-2β,3α-diol steroid 36b.

Introduction of the 17α-ethynyl group according to the procedure of Example 3, Step B into a suitably protected 5α-androstan-17-one-2β,3α-diol or 5α-androstan-17-one-2β,3α-diol so obtained provides 37a or 37b.

Some 3α-hydroxy steroids having a monovalent O-linked moiety at position-2 that may be prepared according to the preceding procedures are the following.

17α-ethynyl-androst-5-ene-2α,3α,7β,17β-tetrol: t_{R}=4.34 min.; δ (ppm) 5.28 (bs, 1H, 5-ene), 3.90 (m, 1H, 3β-H), 3.82 (m, 1H, 2β-H), 3.78 (m, 1H, 7α-H), 2.88 (s, 1H, 17α-C≡CH), 2.55 (m, 1H, 4β-H), 2.20 (dd, 1H, 4α-H), 1.11 (s, 3H, 19β-CH₃), 0.85 (s, 3H, 18β-CH₃).

androst-5-ene-2α,3α,7β,17β-tetrol, t_{R}=3.98 min.; δ (ppm) 5.27 (bs, 1H, 5-ene), 3.90 (m, 1H, 3β-H), 3.83 (m, 1H, 2β-H), 3.80 (m, 1H, 7α-H), 3.57 (t, 1H, 17α-H), 2.55 (m, 1H, 4β-H), 2.20 (dd, 1H, 4α-H), 1.10 (s, 3H, 19β-CH₃), 0.76 (s, 3H, 18β-CH₃).

androst-5-en-17-one-2α,3α-diol, t_{R}=6.59 min.; δ (ppm) 5.40 (m, 1H, 5-ene), 3.87 (m, 1H, 3β-H), 3.82, (m, 1H, 2β-H), 2.53 (m, 1H, 4β-H), 2.45 (dd, 1H, 16β-H), 2.20 (dd, 1H, 4α-H), 1.08 (s, 3H, 19β-CH₃), 0.90 (s, 3H, 18β-CH₃).

5α-androstane-2β,3α,17β-triol: t_{R}=6.20 min.; δ (ppm) 3.79 (bs, 1H, 3β-H), 3.75 (bs, 1H, 2α-H), 3.55 (t, 1H, 17α-H), 1.96 (m, 1H, 1β-H), 0.99 (s, 3H, 19β-CH₃), 0.71 (s, 3H, 18β-CH₃)

17α-methyl-5α-androstane-2β,3α,17β-triol: 2β,3α,17β-triol: t_{R}=6.40 min.; δ (ppm) 3.81 (bs, 1H, 3β-H), 3.75 (bs, 1H, 2α-H), 1.17 (s, 3H, 17α-CH₃), 1.01 (s, 3H, 19β-CH₃), 0.83 (s, 3H, 18β-CH₃).

17α-ethynyl-5α-androstane-2β,3α,17β-triol: 2β,3α,17β-triol: t_{R}=6.75 min.; δ (ppm) 3.80 (bs, 1H, 3β-H), 3.75 (bs, 1H, 2α-H), 2.86 (s, 1H, 17α-C≡CH), 2.19 (m, 1H, 16α-H), 1.92 (td, 1H, 16β-H), 1.01 (s, 3H, 19β-CH3), 0.81 (s, 3H, 18β-CH₃).

5α-androstane-2α,3α,17β-triol: t_{R}=6.57 min.; δ (ppm) 3.88 (bs, 1H, 3β-H), 3.67 (ddd, 1H, 2β-H), 3.56 (t, 1H, 17α-H), 1.96 (m, 1H, 1β-H), 1.72 (dt, 1H, 4α-H), 0.85 (s, 3H, 19β-CH₃), 0.72 (s, 3H, 18β-CH₃).

17α-ethynyl-5α-androstane-2α,3α,17β-triol: t_{R}=7.11 min.; δ (ppm) 3.88 (bs, 1H, 3β-H), 3.67 (dt, 1H, 2β-H), 2.87 (s, 1H, 17α-C≡CH), 2.19 (m, 1H, 16α-H), 1.92 (td, 1H, 16β-H), 0.85 (s, 3H, 19β-CH₃), 0.82 (s, 3H, 18β-CH₃).

HPLC retention times (t_{R}) were obtained using the following conditions. Column: Agilent XDB-C18, 3.5 um, 4.6×150 cm; Mobile phase: A: Water with 0.1% TFA, B: Acetonitrile with 0.1% TFA; Method: 10-90% B in 10 min at ambient temperature. ¹H-NMR data (400 MHz, CD₃OD) is for selected peaks.

What is claimed is:

1. A compound having the structure 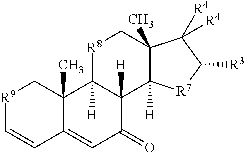
wherein R³ is —H, halogen, a monovalent O-linked moiety or a monovalent C-linked moiety;
one R⁴ is a monovalent O-linked moiety and the other R⁴ is —H, a monovalent O-linked moiety or a monovalent C-linked moiety or both R⁴ together form a divalent O-link moiety;
R⁷ and R⁸ independently are —C(R¹⁰)₂— wherein R¹⁰ independently are —H, a monovalent O-linked, a monovalent C-linked moiety or together are a divalent O-linked moiety;
R⁹ is —C(R¹⁰)₂—, wherein R¹⁰ independently are —H, a monovalent O-linked or a monovalent C-linked moiety;
provided that R³ is halogen, a monovalent O-linked moiety or a monovalent C-linked moiety when R⁹ is —CH₂—.

2. The compound of claim 1 wherein R³ is —H, —Br, —Cl, —F or a monovalent O-linked moiety or a monovalent C-linked moiety, wherein the C-linked moiety is optionally substituted alkyl;
one R⁴ is a monovalent O-linked moiety and the other R⁴ is —H, a monovalent C-linked moiety, wherein the monovalent C-linked moiety is optionally substituted alkyl, optionally substituted alkenyl or optionally substituted alkynyl, or a monovalent O-linked moiety or both R⁴ together form a divalent O-linked moiety wherein the divalent O-linked moiety is ═O, —O—C(R¹⁶)₂—C(R¹⁶)₂—O— or —O—C(R¹⁶)₂—C(R¹⁶)₂—C(R¹⁶)₂—O—, wherein R¹⁶ independently are —H or C₁₋₄ alkyl or two of R¹⁶ and the carbon(s) to which they are attached comprise an optionally substituted C₃, C₅ or C₆ cycloalkyl, and the other R¹⁶ are —H;
R⁷ and R⁸ are —CH₂—;
R⁹ is —C(R¹⁰)₂—, wherein one R¹⁰ is —H and the other R¹⁰ is —H or a monovalent O-linked moiety or R⁹ is —CH₂—, —CH(α-OH)— or —CH(β-OH)—;
wherein the monovalent O-linked moieties, independently selected, are —OH, —OR^{PR}, wherein R^{PR} is a hydroxyl protecting group, an ester, an ether or a silyl ether.

3. The compound of claim 1 wherein the compound is 17,17-ethylenedioxy-16α-fluoro-androst-3,5-dien-7-one, 17,17-ethylenedioxy-androst-3,5-dien-7-one-2α-ol, androst-3,5-dien-7,17-dione-16α-ol, 2α-acetoxy-androst-3,5-dien-7,17-dione, androst-3,5-dien-7,17-dione-2α-ol, 16α-fluoro-androst-3,5-dien-7,17-dione, 16α-methoxy-androst-3,5-dien-7,17-dione, 16α-methyl-androst-3,5-dien-7,17-dione or 16α-propyl-androst-3,5-dien-7,17-dione.

4. A compound having the structure 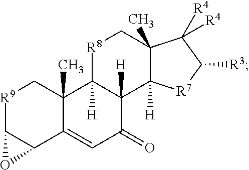
wherein R³ is —H, halogen, a monovalent O-linked moiety or a monovalent C-linked moiety;
one R⁴ is a monovalent O-linked moiety and the other R⁴ is —H, a monovalent O-linked moiety or a monovalent C-linked moiety or both R⁴ together are a divalent O-linked moiety;
R⁷ and R⁸ independently are —C(R¹⁰)₂— wherein R¹⁰ independently are —H, a monovalent O-linked, a monovalent C-linked moiety or together form a divalent O-linked moiety;
R⁹ is —C(R¹⁰)₂—, wherein R¹⁰ independently are —H, a monovalent O-linked moiety, a monovalent C-linked moiety;
provided that R³ is halogen, a monovalent O-linked moiety or a monovalent C-linked moiety when R⁷, R⁸ and R⁹ are —CH₂— and both R⁴ together are ═O.

5. The compound of claim 4 wherein R³ is —H, halogen, optionally bromo, chloro or fluoro, or a monovalent O-linked moiety or a monovalent C-linked moiety, wherein the C-linked moiety is optionally substituted alkyl;
one R⁴ is a monovalent O-linked moiety and the other R⁴ is —H, a monovalent C-linked moiety, wherein the monovalent C-linked moiety is optionally substituted alkyl, optionally substituted alkenyl or optionally substituted alkynyl, or a monovalent O-linked moiety or both R⁴ together form a divalent O-linked moiety wherein the divalent O-linked moiety is ═O, —O—C(R¹⁶)₂—C(R¹⁶)₂—O— or —O—C(R¹⁶)₂—C(R¹⁶)₂—C(R¹⁶)₂—O—, wherein R¹⁶ independently are —H or C₁₋₄ alkyl or two of R¹⁶ and the carbon(s) to which they are attached comprise an optionally substituted C₃, C₅ or C₆ cycloalkyl, and the other R¹⁶ are —H;
R⁷ and R⁸ are —CH₂—;
R⁹ is —C(R¹⁰)₂— wherein one R¹⁰ is —H and the other R¹⁰ is —H or a monovalent O-linked moiety or R⁹ is —CH₂—, —CH(α-OH)— or —CH(β-OH)—;
wherein the monovalent O-linked moieties, independently selected, are —OH, an ester, an ether or a silyl ether.

6. The compound of claim 4 wherein the compound has the structure 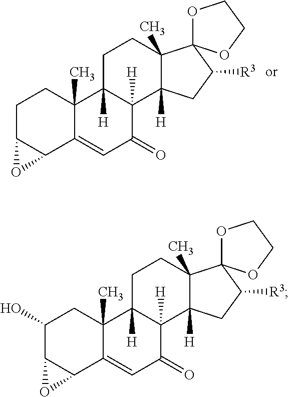
wherein R³ is —H, fluoro, C₁₋₄ alkyl, C₁₋₄ ether, C₁₋₄ ester or a silyl ether.

7. The compound of claim 4 wherein the compound is prepared by a process comprising the step of contacting a suitably protected androst-3,5-diene of claim 1 with an epoxidizing agent wherein the epoxidizing agent selectively reacts with the Δ³ functional group relative to the Δ⁵ functional group, wherein a 3α,4α-epoxy-androst-5-en-7-one steroid product is obtained.

8. The compound of claim 7 wherein the optionally deprotected compound is 17,17-ethylenedioxy-3α,4α-epoxy-androst-5-en-7-one, 17,17-ethylenedioxy-3α,4α-epoxy-androst-5-en-7-one-2α-ol, 3α,4α-epoxy-androst-5-en-7,17-dione-16α-ol, 2α-acetoxy-3α,4α-epoxy-androst-5-en-7,17-dione, 3α,4α-epoxy-androst-5-en-7,17-dione-2α-ol, 16α-fluoro-3α,4α-epoxy-androst-5-en-7,17-dione, 16α-methoxy-3α,4α-epoxy-androst-5-en-7,17-dione, 16α-methyl-3α,4α-epoxy-androst-5-en-7,17-one or 16α-propyl-3α,4α-epoxy-androst-5-en-7,17-one.

9. A process to prepare a 3α-O-linked androst-5-ene steroid comprising the step of
(1) contacting a suitably protected 3α,4α-epoxy-androst-5-ene with a first hydrogen donor,
wherein the 3α,4α epoxy functional group is selectively reduced relative to the Δ⁵ functional group and wherein reduction of the 3α,4α epoxy functional group occurs preferentially at position C-4 with retention of configuration at position position C-3,
wherein the suitably protected 3α,4α-epoxy-androst-5-ene has the structure 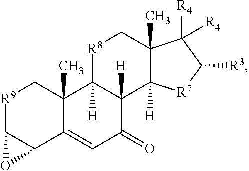
wherein R³ is —H, a suitable halogen, a suitable monovalent O-linked moiety or a suitable monovalent C-linked moiety; and
R⁴ independently are an ether or both R⁴ together are —OC(R¹⁶)₂C(R¹⁶)₂O— (ketal), wherein R¹⁶ independently are —H or C₁₋₄ alkyl or two of R¹⁶ and the carbon(s) to which they are attached form a C₃, C₅ or C₆ cycloalkyl, and the remaining R¹⁶ are —H; and
R⁹, R⁷ and R⁸ independently are —C(R¹⁰)₂, wherein R¹⁰ independently are —H or a suitable monovalent O-linked moiety.

10. The process of claim 9 wherein the first hydrogen donor selectively reduces the 3α,4α epoxy functional group in preference to the C-7 ketone functional group, whereby a 3α-O-linked androst-5-ene product having a ═O (ketone) moiety at position C-7 is obtained.

11. The process of claim 9 further comprising the step of
(2) contacting the product obtained or prepared from step (1) with an electrophile, wherein a monovalent O-linked group is obtained at position C-3, wherein the monovalent O-linked group so obtained is other than —OH.

12. The process of claim 9 further comprising the step of
(3) contacting a suitably protected 3α-O-linked androst-5-en-7-one obtained or prepared from the 3α-O-linked androst-5-ene product of step (1) with a second hydrogen donor,
wherein the suitably protected 3α-O-linked androst-5-en-7-one has the structure 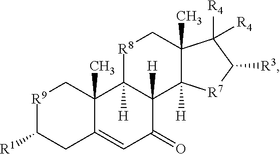
wherein R¹ is a suitable monovalent O-linked moiety; R³ is —H, a suitable C-linked moiety, a suitable halogen or a suitable monovalent O-linked moiety;
R⁴ independently are an ether or one R⁴ is a suitable monovalent O-linked moiety and the other R⁴ is —H or both R⁴ together are ═O (ketone) or —OC(R¹⁶)₂C(R¹⁶)₂O— (ketal), wherein R¹⁶ independently are —H or C₁₋₄ alkyl or two of R¹⁶ and the carbon(s) to which they are attached form a C₃, C₅ or C₆ cycloalkyl, and the remaining R¹⁶ are —H;
wherein a 3α-O-linked androst-5-ene product having —OH in the α- or β-configuration at position C7 is obtained.

13. The process of claim 12 wherein the suitably protected 3α-O-linked androst-5-en-7-one contacted with the second hydrogen donor has the structure

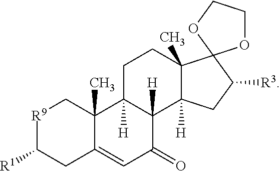

14. The process of claim 9 wherein the first hydrogen donor is provided by Pd(0)/H₂, wherein the palladium catalyst is supported on carbon black and is suspended in an alcohol-based solvent in the presence of a carbonate salt to which is applied
a hydrogenation temperature of between about ambient or about 40° C. or about 22° C. to about 40° C. and a hydrogenation pressure of between about 15.5 psi to about 50 psi H₂,
wherein the 3α,4α-epoxy functionality is selectively reduced relative to the C7 ketone functional group and whereby reduction of the 3α,4α epoxy functional group occurs preferentially at position C4 with retention of configuration at position C3.

15. The process of claim 14 wherein the hydrogenation temperature is ambient or about 22° C., the hydrogenation pressure is about 22 psi H₂, the carbonate salt is potassium carbonate and the alcohol-based solvent is a mixture of ethanol and ethyl acetate in about 5:1 by volume ratio.

16. The process of claim 13 wherein the second hydrogen donor is a suitable hydride reducing agent.

17. The process of claim 9 wherein the suitable monovalent O-linked moieties independently are an ether, —OSi(R¹³)₃, or —OR^{PR}, wherein R^{PR} is —H, a protecting group and R¹³ independently are C₁₋₄ alkyl or aryl, the suitable halogen in R³ is fluoro; and the suitable monovalent C-linked moiety is optionally substituted alkyl, suitably protected.

18. The process of claim 9 wherein the suitably protected 3α,4α-epoxy-androst-5-ene is 17,17-ethylenedioxy-3α,4α-epoxy-androst-5-en-7-one, 17,17-di-methoxy-3α,4α-epoxy-androst-5-en-7-one, 17,17-di-ethoxy-3α,4α-epoxy-androst-5-en-7-one, 17,17-(propylene-1,3-dioxy)-3α,4α-epoxy-androst-5-en-7-one, 17,17-tetramethyl-ethylenedioxy-3α,4α-epoxy-androst-5-en-7-one, 17,17-(cyclohex-1,2-yl)-dioxy-3α,4α-epoxy-androst-5-en-7-one, 17,17-ethylenedioxy-16α-methoxy-3α,4α-epoxy-androst-5-en-7-one, 17,17-ethylenedioxy-16α-fluoro-3α,4α-epoxy-androst-5-en-7-one, 17,17-ethylenedioxy-16α-trimethylsilyloxy-3α,4α-epoxy-androst-5-en-7-one or 17,17-ethylenedioxy-16α-(t-butyl-dimethylsilyl)oxy-3α,4α-epoxy-androst-5-en-7-one.

19. The process of any one of claims 9-12 wherein the 3α-O-linked-androst-5-ene steroid prepared, optionally after protecting group removal, has the structure 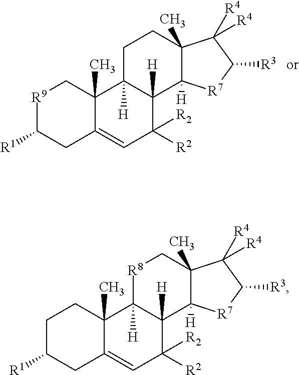
wherein R¹ is —OH, —OR¹¹, —OC(O)—R¹² or —OSi(R¹³)₃;
one of R² is —OH, —OR¹¹, —OC(O)—R¹² or —OSi(R¹³)₃ and the other R² is —H or both R² together are ═O;
R³ is —H, —OH, —OR¹¹, —OC(O)—R¹²—OSi(R¹³)₃, halogen or C₁₋₄ alkyl;
R⁴ independently or together are —OH, —OR¹¹, —OC(O)—R¹², —OSi(R¹³)₃, ═O or —OC(R¹⁶)₂C(R¹⁶)₂O—;
R⁷ and R⁸ independently are —C(R¹⁰)₂— wherein both R¹⁰ are —H or one R¹⁰ is α-OH—, β-OH, α-ester, or β-ester and the other R¹⁰ is —H;
R⁹ is —C(R¹⁰)₂—, wherein one R¹⁰ is α-OH, β-OH, α-ester or β-ester and the other R¹⁰ is —H;
R¹¹, R¹² and R¹³ independently are optionally substituted C₁₋₆ alkyl or optionally substituted aryl; and
R¹⁶ independently are —H or C₁₋₄ alkyl or two of R¹⁶ and the carbon(s) to which they are attached form a C₃, C₅ or C₆ cycloalkyl, and the remaining R¹⁶ are —H;
wherein the optionally substituted C₁₋₆ alkyl of each R¹¹, independently selected, is —CH₃ or —CH₂CH₃;
wherein each R¹², independently selected, is —CH₃ or phenyl or two of R¹³ in each —OSi(R¹³)₃, independently selected, are —CH₃ or —CH₂CH₃ and the remaining R¹³ is —CH₃, —CH₂CH₃, t-butyl or phenyl.

20. The process of claim 19 wherein the 3α-O-linked-androst-5-ene steroid prepared has the structure

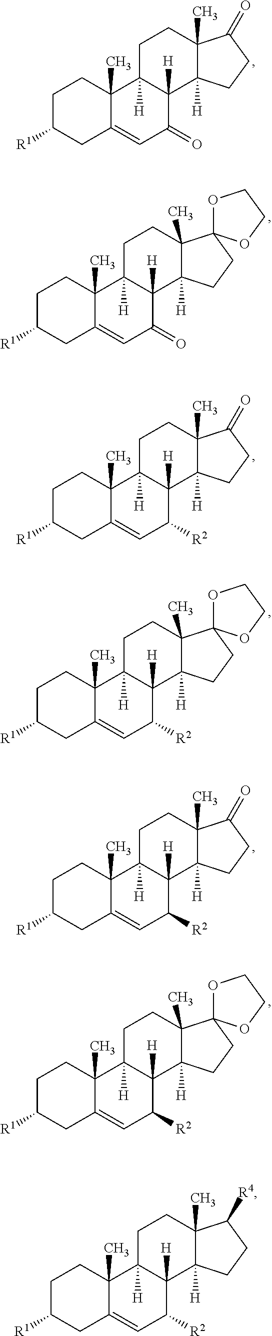

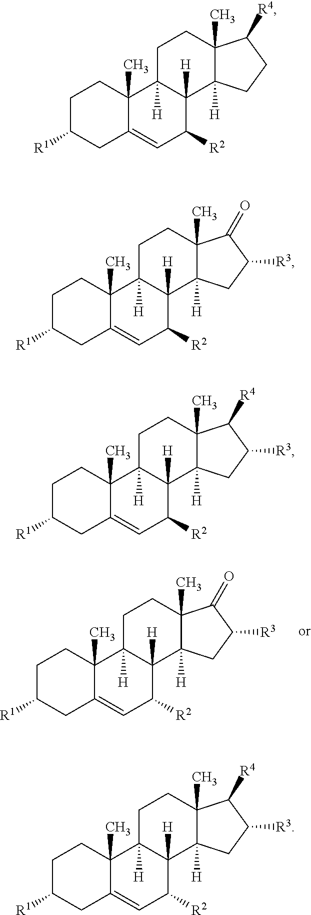

21. The process of claim 19 wherein the 3α-O-linked androst-5-ene steroid prepared, optionally after protecting group removal, is androst-5-en-7,17-dione-3α-ol, 3α-acetoxy-androst-5-en-7,17-dione, 17,17-ethylenedioxy-androst-5-en-7-one-3α-ol, 17,17-ethylenedioxy-3α-acetoxy androst-5-en-7-one, androst-5-en-17-one-3α,713-diol, 3α-acetoxy-androst-5-en-17-one-7β-ol, androst-5-en-17-one-3α,7α-diol, 3α-acetoxy-androst-5-en-17-one-7α-ol, 17,17-ethylenedioxy-androst-5-ene-3α,713-diol, 17,17-ethylenedioxy-3α-acetoxy-androst-5-ene-7β-ol, 17,17-ethylenedioxy-androst-5-ene-3α,7α-diol, 17,17-ethylenedioxy-3α-acetoxy-androst-5-ene-7α-ol, androst-5-en-17-one-3α,713,16α-triol, 16α-methoxy-androst-5-en-17-one-3α,713-diol, 16α-fluoro-androst-5-en-17-one-3α,713-diol, androst-5-ene-3α,7β,16α,17β-tetrol, 16α-methoxy-androst-5-ene-3α,7β,17β-triol, 16α-fluoro-androst-5-ene-3α,7β,17β-triol, androst-5-en-17-one-3α,7α,16α-triol, 16α-methoxy-androst-5-en-17-one-3α,7α-diol, 16α-fluoro-androst-5-en-17-one-3α,7α-diol, androst-5-ene-3α,7α,16α,17β-tetrol, 16α-methoxy-androst-5-ene-3α,7α,17β-triol or 16α-fluoro-androst-5-ene-3α,7α,17β-triol.

22. The process of claim 9, further comprising the step of
(5) contacting a suitably protected 3α-O-linked androst-5-ene prepared or obtained from the 3α-O-linked androst-5-ene product with a third hydrogen donor to reduce the Δ⁵ functional group, wherein a 3α-O-linked-5α-androstane product is obtained.

23. The process of claim 22 wherein the 3α-O-linked-5α-androstane steroid prepared, optionally after protecting group removal, has the structure 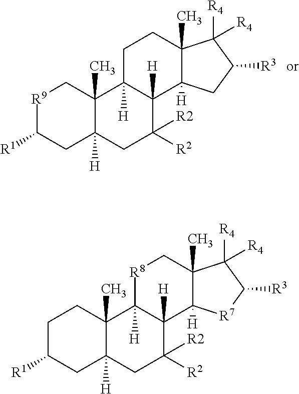
wherein R¹ is —OH, —OR¹¹, —OC(O)—R¹² or —OSi(R¹³)₃;
one of R² is —OH, —OR¹¹, —OC(O)—R¹² or —OSi(R¹³)₃ and the other R² is —H or both R² together are ═O;
R³ is —H, —OH, —OR¹¹, —OC(O)—R¹²—OSi(R¹³)₃, halogen or C₁₋₄ alkyl;
R⁴ independently or together are —OH, —OR¹¹, —OC(O)—R¹², —OSi(R¹³)₃, ═O or —OC(R¹⁶)₂C(R¹⁶)₂O—;
R⁷ and R⁸ independently are —C(R¹⁰)₂— wherein both R¹⁰ are —H or one R¹⁰ is α-OH—, β-OH, α-ester, or β-ester and the other R¹⁰ is —H;
R⁹ is —C(R¹⁰)₂—, wherein one R¹⁰ is α-OH, β-OH, α-ester or β-ester and the other R¹⁰ is —H;
R¹¹, R¹² and R¹³ independently are optionally substituted C₁₋₆ alkyl or optionally substituted aryl or each R¹², independently selected, is —CH₃ or phenyl, two of R¹³ in each —OSi(R¹³)₃, independently selected, are —CH₃ or —CH₂CH₃ and the remaining R¹³ is —CH₃, —CH₂CH₃, t-butyl or phenyl; and
R¹⁶ independently are —H or C₁₋₄ alkyl or two of R¹⁶ and the carbon(s) to which they are attached form a cycloalkyl, optionally C₃, C₅ or C₆ cycloalkyl, and the remaining R¹⁶ are —H.

24. The process of claim 23 wherein (i) R⁷ and R⁸ are —CH₂—, (ii) R⁷ is —CH(α-OH)— or —CH(β-OH)— and R⁸ is —CH₂— or (iii) R⁷ is —CH₂— and R⁸ is —CH(β-OH)—;
R⁹ is —CH(α-OH);
the optionally substituted C₁₋₆ alkyl of each R¹¹, independently selected, is —CH₃ or —CH₂CH₃, each R¹², independently selected, is —CH₃ or phenyl and two of R¹³ in each —OSi(R¹³)₃, independently selected, are —CH₃ or —CH₂CH₃ and the remaining R¹³ is —CH₃, —CH₂CH₃, t-butyl or phenyl or R¹² and R¹³ are —CH₃ or R¹² is —CH₃ and two of R¹³ are —CH₃ or —CH₂CH₃ and the remaining R¹³ is —CH₂CH₃, t-butyl or phenyl.

25. The process of claim 22, optionally after protecting group removal, wherein the 3α-O-linked-5α-androstane steroid prepared has the structure

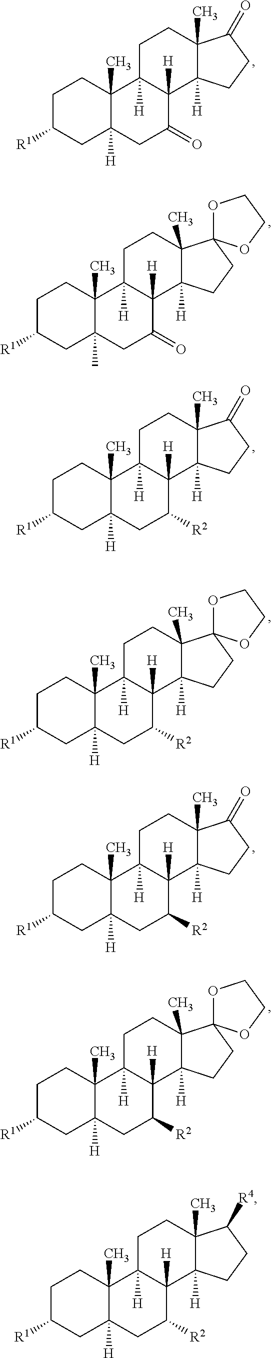

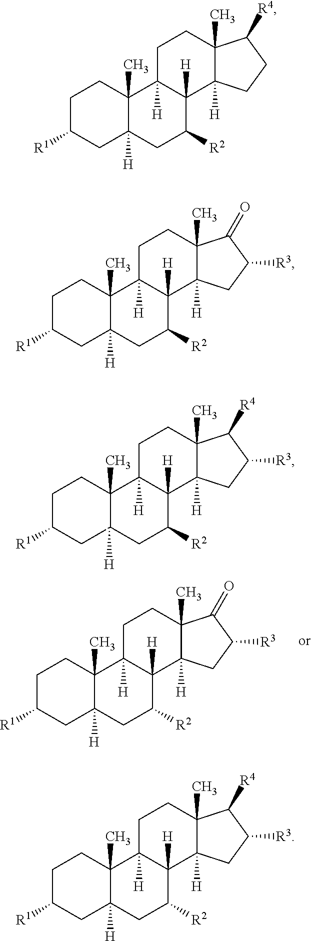

26. The process of claim 22 wherein the 3α-O-linked-5α-androstane steroid prepared, optionally after protecting group removal, is 5α-androstan-7,17-dione-3α-ol, 3α-acetoxy-5α-androstan-7,17-dione, 17,17-ethylenedioxy-5α-androstan-7-one-3α-ol, 17,17-ethylenedioxy-3α-acetoxy-5α-androstan-7-one, 5α-androstan-17-one-3α,7α-diol, 17,17-ethylenedioxy-5α-androstane-3α,7α-diol, 5α-androstan-17-one-3α,713-diol, 17,17-ethylenedioxy-5α-androstane-3α,7β-diol, 5α-androstane-3α,7α,17β-triol, 5α-androstane-3α,7β,17β-triol, 5α-androstane-3α,7α,16α,17β-tetrol, 5α-androstane-3α,7β,16α,17β-tetrol, 16α-fluoro-5α-androstane-3α,7β,17β-triol, 16α-methoxy-5α-androstane-3α,7β,17β-triol, 16α-methyl-5α-androstane-3α,7β,17β-triol or 16α-propyl-5α-androstane-3α,7β,17β-triol.

27. The process of claim 9 further comprising the step of
(6a) contacting a suitably protected 3α-O-linked-androst-5-ene, obtained or prepared from the 3α-O-linked-androst-5-ene product having a ═O moiety (ketone) after deprotection at position C-17 with a suitably protected optionally substituted alkyl, optionally substituted alkenyl or optionally substituted alkynyl organometallic anion, wherein the organometallic anion adds to the ═O moiety;
wherein a 3α-O-linked androst-5-ene steroid product having disubstitution at position C-17 is prepared.

28. The process of claim 27 wherein the organometallic anion has the structure of M-C≡C—Si(R¹³)₃,
wherein R¹³ independently are C₁₋₆ alkyl or aryl or R¹³ are —CH3; and
wherein M is a Group I, Group II or transition metal or is Na, Li, Mg or Zn.

29. The process of claim 27 wherein the C17-disubstituted 3α-O-linked androst-5-ene steroid prepared, optionally after protecting group removal, has the structure 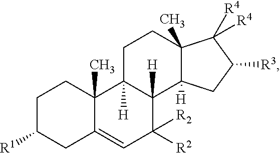
wherein
R¹ is —OH, —OR^{PR}, —OR¹¹, —OC(O)—R¹² or —OSi(R¹³)₃;
one of R² is —OH, —OR^{PR}, —OR¹¹, —OC(O)—R¹² or —OSi(R¹³)₃ and the other R² is —H or both R² together are ═O;
R³ is —H, —OH, —OR^{PR}, —OR¹¹, —OC(O)—R¹², fluoro or optionally substituted alkyl;
one R⁴ is —OH, —OR¹¹, —OC(O)—R¹², —OSi(R¹³)₃ and the other R⁴ is an optionally substituted alkynyl, wherein the optionally substituted alkynyl has the structure —C≡R, wherein R is CR^{A} and wherein R^{A} is H, optionally substituted alkyl or —Si(R¹³)₃;
wherein (i) R¹¹, R¹² and R¹³ independently are optionally substituted C₁₋₆ alkyl or optionally substituted aryl or (ii) each R¹¹, independently selected, is —CH₃ or —CH₂CH₃, each R¹², independently selected, is —CH₃ or phenyl and two of R¹³ in each —OSi(R¹³)₃, independently selected, are —CH₃ or —CH₂CH₃ and the remaining R¹³ are —CH₃, —CH₂CH₃, t-butyl or phenyl.

30. The process of claim 29 wherein the C17 di-substituted 3α-O-linked androst-5-ene steroid prepared, optionally after protecting group, removal has the structure 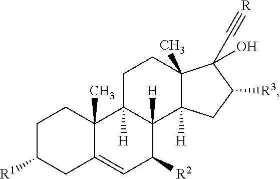
wherein R¹ and R² independently are —OH or —OSi(R¹³)₃;
R³ is —H, —OH or —OSi(R¹³)₃ and R in —C≡R is CR^{A} wherein R^{A} is —H, optionally substituted C₁₋₆ alkyl or —Si(R¹³)₃;
wherein (i) R¹³ independently are C₁₋₆ alkyl or aryl or (ii) two of R¹³ in one or more of —OSi(R¹³)₃ or in —Si(R¹³)₃ are —CH₃ or —CH₂CH₃ and the remaining R¹³ are —CH₃, —CH₂CH₃, t-butyl or phenyl, independently selected.

31. The process of claim 30 wherein R¹ and R² independently are —OH or —OSi(R¹³)₃ wherein R¹³ are —CH₃; R³ is —H and R^{A} is —Si(CH₃)₃.

32. The process of claim 27 wherein the 3α-O-linked androst-5-ene steroid prepared, optionally after protecting group removal, is 17α-ethynyl-androst-5-ene-3α,7β,17β-triol, 17α-ethynyl-androst-5-ene-3α,7α,17β-triol, 17α-ethynyl-androst-5-ene-3α,76,16α,17β-tetrol, 17α-ethynyl-androst-5-ene-3α,7α,16α,17β-tetrol, 17α-ethenyl-androst-5-ene-3α,7β,17β-triol, 17α-methyl-androst-5-ene-3α,76,16α,17β-tetrol, 17α-ethynyl-16α-fluoro-androst-5-ene-3α,7β,17β-triol or 17α-ethynyl-16α-methoxy-androst-5-ene-3α,7β,17β-triol.

33. The process of claim 22 further comprising the step of
(6b) contacting a suitably protected 3α-O-linked-5α-androstane, obtained or prepared from the 3α-O-linked-androst-5-ene product having a ═O moiety (ketone) after deprotection at position C-17, with a suitably protected optionally substituted alkyl, optionally substituted alkenyl or optionally substituted alkynyl organometallic anion; wherein the organometallic anion adds to the ═O moiety;
wherein a 3α-O-linked 5α-androstane steroid product having disubstitution at position C-17 is prepared.

34. The process of claim 33 wherein the organometallic anion has the structure of M-C≡C—Si(R¹³)₃,
wherein R¹³ independently are C₁₋₆ alkyl or aryl or R¹³ are —CH₃;
wherein M is a Group I, Group II or transition metal or is Na, Li, Mg or Zn.

36. The process of claim 33 wherein the C17-disubstituted 3α-O-linked 5α-androstane steroid prepared, optionally after protecting group removal, has the structure 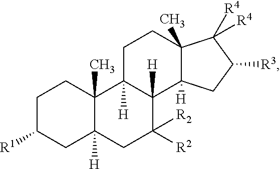
wherein
R¹ is —OH, —OR^{PR}, —OR¹¹, —OC(O)—R¹² or —OSi(R¹³)₃;
one of R² is —OH, —OR^{PR}, —OR¹¹, —OC(O)—R¹² or —OSi(R¹³)₃ and the other R² is —H or both R² together are ═O;
R³ is —H, —OH, —OR^{PR}, —OR¹¹, —OC(O)—R¹², fluoro or optionally substituted alkyl;
one R⁴ is —OH, —OR¹¹, —OC(O)—R¹², —OSi(R¹³)₃ and the other R⁴ is an optionally substituted alkynyl wherein the optionally substituted alkynyl has the structure —C≡R; wherein R is CR^{A} and wherein R^{A} is H, optionally substituted alkyl or —Si(R¹³)₃;
wherein (i) R¹¹, R¹² and R¹³ independently are optionally substituted C₁₋₆ alkyl or optionally substituted aryl or (ii) each R¹¹, independently selected, is —CH₃ or —CH₂CH₃, each R¹², independently selected, is —CH₃ or phenyl and two of R¹³ in each —OSi(R₁₃)₃, independently selected, are —CH₃ or —CH₂CH₃ and the remaining R¹³ are —CH₃, —CH₂CH₃, t-butyl or phenyl.

37. The process of claim 33 wherein the C17 di-substituted 3α-O-linked 5α-androstane steroid prepared, optionally after protecting group, removal has the structure 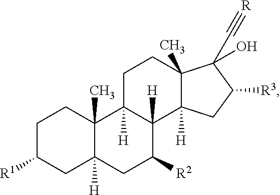
wherein R¹ and R² independently are —OH or —OSi(R¹³)₃; and
R³ is —H, —OH or —OSi(R¹³)₃ and R in —C≡R is CR^{A}, wherein R^{A} is —H, optionally substituted C₁₋₆ alkyl or —Si(R¹³)₃;
wherein (i) R¹³ independently are C₁₋₆ alkyl or aryl or (ii) two of R¹³ in one or more of —OSi(R¹³)₃ or in —Si(R¹³)₃ are —CH₃ or —CH₂CH₃ and the remaining R¹³ are —CH₃, —CH₂CH₃, t-butyl or phenyl, independently selected.

38. The process of claim 37 wherein R¹ and R² independently are —OH or —OSi(R¹³)₃ wherein R¹³ are —CH₃, R³ is —H and R^{A} is —Si(CH₃)₃.

39. The process of claim 33 wherein the C17 di-substituted 3α-O-linked 5α-androstane steroid prepared, optionally after protecting group removal, is 17α-ethynyl-androst-5-ene-3α,17β-diol, 17α-ethynyl-5α-androstane-3α,17β-diol, 17α-ethenyl-5α-androstane-3α,17β-diol, 17α-ethyl-5α-androstane-3α,17β-diol, 17α-methyl-androst-5-ene-3α,17β-diol, 17α-ethynyl-16α-fluoro-5α-androstane-3α,17β-diol, 17α-ethynyl-16α-methoxy-5α-androstane-3α,17β-diol, 17α-ethynyl-16α-fluoro-androst-5-ene-3α,17β-diol, 17α-ethynyl-androst-5-ene-3α,16α,17β-triol or 17α-ethynyl-5α-androstane-3α,16α,17β-triol.

40. A process to prepare a 3α-O-linked-androst-5-ene steroid comprising,
(1) contacting a suitably protected 3β-hydroxy steroid with an azo-di-carboxylate ester, a tri-substituted phosphine and an organic acid having the structure of R¹²C(O)OH wherein R¹² is C₁₋₆ alkyl, C₃₋₆ cycloalkyl or optionally substituted aryl, wherein the suitably protected 3β-hydroxy steroid has the structure 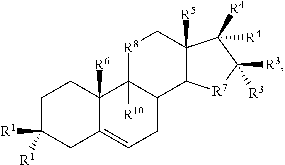
wherein R¹ in the β-configuration is —OH and R¹ in the α-configuration is —H or a suitable optionally substituted alkyl;
R³ independently or together are —H, halogen, a suitable C-linked moiety, a suitable monovalent O-linked moiety, ═O (ketone) or —O—C(R¹⁶)₂—C(R¹⁶)₂—O— (ketal);
R⁴ in the β-configuration is a suitable monovalent O-linked moiety; R⁴ in the α-configuration is —H or a suitable C-linked moiety or R⁴ together are ═O (ketone) or —O—C(R¹⁶)₂—C(R¹⁶)₂—O— (ketal), wherein R¹⁶ independently are —H or C₁₋₄ alkyl or two of R¹⁶ and the carbon(s) to which they are attached form an optionally substituted C₃, C₅ or C₆ cycloalkyl or C₃, C₅ or C₆ spiroalkyl;
R⁵ and R⁶ independently are —H or a suitable optionally substituted alkyl;
R⁷ and R⁸ independently are —C(R¹⁰)₂—; wherein R¹⁰ independently or together are —H, a suitable halogen, a suitable monovalent C-linked moiety or a suitable monovalent O-linked moiety or both R¹⁰ together are ═O or —O—C(R¹⁶)₂—C(R¹⁶)₂—O— (ketal);
R¹⁰ at position C-9 is —H or halogen;
R^{PR} independently are —H or protecting group;
wherein the C-linked moieties are independently a suitable optionally substituted alkyl group, optionally substituted alkenyl group or optionally substituted alkynyl group; and
wherein the monovalent O-linked moieties independently are —OR^{PR} an ester or an ether;
wherein the molar ratio of the azo-di-carboxylate ester to the 3β-hydroxy steroid is less than 1.5:1 and greater than 1.0:1;
wherein a 3α-androst-5-ene product having a 3α-O-linked ester substantially free of 3α,5α-cycloandrostane side-products is obtained.

41. The process of claim 40 wherein the molar ratio of the azo-di-carboxylate ester to the 3β-hydroxy steroid is about 1.3:1 and the tri-substituted phosphine and organic acid are in substantially equimolar amounts relative to the azo-di-carboxylate ester.

42. The process of claim 40 wherein wherein the an azo-di-carboxylate ester is added to a mixture of the tri-substituted phosphine, organic acid and β-hydroxy steroid at between about 0 to 25° C.

43. The process of claim 42 wherein the azo-di-carboxylate ester is added to a mixture of the tri-substituted phosphine at a temperature of between about 0-10° C. whereupon the mixture is warmed to between about 10-25° C.

44. The process of claim 40 wherein R¹⁹ is p-NO₂-phenyl and the azo-di-carboxylate ester has the structure R¹⁹OC(O)N═NC(O)OR¹⁹ wherein R¹⁹ is —CH₂CH₃ (DEAD) or —CH(CH₃)₂ (DIAD).

45. The process of claim 40 wherein 3α-O-linked-androst-5-ene steroid prepared, optionally after protecting group removal, has the structure 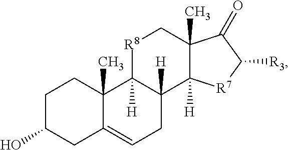
wherein R³ is —H, halogen, a monovalent O-linked moiety or a monovalent C-linked moiety; R⁷ and R⁸ independently are —C(R¹⁰)₂ wherein R¹⁰ independently are —H a monovalent O-linked moiety or a monovalent C-linked moiety.

46. The process of claim 40 wherein 3α-O-linked-androst-5-ene steroid prepared, optionally after protecting group removal, is androst-5-en-17-one-3α-ol (3α-DHEA), androst-5-en-17-one-3α,11β-diol, androst-5-en-17-one-3α,15α-diol, androst-5-en-17-one-3α,15α,16α-triol, androst-5-en-17-one-3α,1113,16α-triol, 16α-fluoro-androst-5-en-17-one-3α-ol.

47. The process of claim 40 further comprising the step of
(3) contacting a suitably protected 3α-O-linked androst-5-ene prepared or obtained from the 3α-O-linked-androst-5-ene product of claim 43 with a hydrogen donor to reduce the Δ⁵ functional group, wherein a 3α-O-linked-5α-androstane product is obtained.

48. The process of claim 40 or 47 further comprising the step of
(4) contacting a suitably protected 3α-O-linked-androst-5-ene obtained or prepared from the 3α-O-linked-androst-5-ene product, having a ═O moiety (ketone) at position C17 of claim 43 or a suitably protected 3α-O-linked-5α-androstane obtained or prepared from the 3α-O-linked-5α-androstane steroid product of claim 53, having a ═O moiety (ketone) at position C17, with a suitably protected optionally substituted alkyl, an optionally substituted alkenyl or an optionally substituted alkynyl organometallic anion, wherein the organometallic anion adds to the ═O moiety to provide a 3α-O-linked 5α-androstane product or a 3α-O-linked 5α-androstane product having disubstitution at position C17.

49. The process of claim 49 wherein the organometallic anion has the structure of M-C≡C—Si(R¹³)₃, wherein R¹³ independently are C₁₋₆ alkyl or aryl and M is a Group I, Group II or transition metal or is Na, Li, Mg or Zn.

50. The process of claim 49 wherein the 3α-O-linked androst-5-ene steroid or the 3α-O-linked 5α-androstane steroid prepared, optionally after protecting group removal, is 17α-ethynyl-androst-5-ene-3α,17β-diol, 17α-ethynyl-5α-androstane-3α,17β-diol, 17α-ethenyl-5α-androstane-3α,17β-diol, 17α-ethyl-5α-androstane-3α,17β-diol, 17α-methyl-androst-5-ene-3α,17β-diol, 17α-ethynyl-16α-fluoro-5α-androstane-3α,17β-diol, 17α-ethynyl-16α-methoxy-5α-androstane-3α,17β-diol, 17α-ethynyl-16α-fluoro-androst-5-ene-3α,17β-diol, 17α-ethynyl-androst-5-ene-3α,16α,17β-triol or 17α-ethynyl-5α-androstane-3α,16α,17β-triol.
